(19) United States
(12) Patent Application Publication
(10) Pub. No.: US 2024/0229047 A1
(43) Pub. Date: Jul. 11, 2024

(54) CARBOXYLIC ACID PLATFORM FOR FUEL AND CHEMICAL PRODUCTION AT HIGH CARBON AND ENERGY EFFICIENCY

(71) Applicant: Mojia Biotech Pte. Ltd., Singapore (SG)

(72) Inventors: Ramon Gonzalez, Tampa, FL (US); James MacAllister Clomburg, Houston, TX (US); Fayin Zhu, Tampa, FL (US); Seung Hwan Lee, Tampa, FL (US); Mohammadreza Nezamirad, Tampa, FL (US)

(21) Appl. No.: 18/406,738

(22) Filed: Jan. 8, 2024

Related U.S. Application Data
(63) Continuation of application No. PCT/US2022/036856, filed on Jul. 12, 2022.
(60) Provisional application No. 63/220,927, filed on Jul. 12, 2021.

(51) Int. Cl. C12N 15/52; C12N 9/00; C12N 9/02; C12N 9/04; C12N 9/10; C12N 9/12; C12N 9/16; C12N 9/88; C12P 1/02; C12P 1/04

(52) U.S. Cl. CPC C12N 15/52; C12N 9/0006; C12N 9/0008; C12N 9/0093; C12N 9/1029; C12N 9/1217; C12N 9/16; C12N 9/88; C12N 9/93; C12P 1/02; C12P 1/04; C12Y 101/01001; C12Y 102/01003; C12Y 207/02006; C12Y 301/0202; C12Y 402/01028

(57) ABSTRACT

This disclosure provides a new conceptual framework in which orthogonal, new-to-nature carbon and energy conversion pathways facilitate the synthesis of fuels and chemicals from carboxylic acid intermediates (CAis) driven by genetically altered microorganisms. This allows the CAi platform to generate diverse products at ≥100% carbon yield while retaining the established high product and energy efficiencies of fermentative metabolism. In another embodiment, a carboxylic acid platform for fuel and chemical production at high carbon and energy efficiency is also provided.

### CROSS-REFERENCE TO RELATED APPLICATIONS

This application is a continuation of International Application No. PCT/US22/036856, which designated the United States and was filed on Jul. 12, 2022, published in English, which claims priority to U.S. Provisional Patent Application No. 63/220,927, filed Jul. 12, 2021, the entire contents of which are hereby incorporated by reference.

### STATEMENT REGARDING FEDERALLY SPONSORED RESEARCH

This invention was made with government support under Award No. DE-AR0001508 and DE-EE0008499 awarded by the Department of Energy. The government has certain rights in the invention.

### REFERENCE TO A SEQUENCE LISTING

N/A

### BACKGROUND

Fermentative metabolism affords high product fluxes and energy efficiencies through both C-conserving and CO2-evolving pathways. With sugars as the sole source of carbon and energy, C-conserving pathways have been exploited for the synthesis of neutral or oxidized products (e.g., carboxylic acids), while CO2-evolving pathways are used for reduced products, which include most industrial chemicals and fuels (e.g. alcohols). However, with the availability of electricity/external reducing equivalents, a paradigm shift is needed to focus on C-conservation opposed to the E-conservation often required by metabolism. To develop carbon optimized fermentation systems for the conversion of sugars, cellular metabolism must be re-engineered to focus on carbon conservation over its evolved prioritization of energy conservation.

Current efforts in this area have focused on either recovering lost carbon from CO2-evolving pathways or re-wiring central metabolism to conserve carbon and prevent carbon losses in the form of CO2 (Bogorad, I., et al. Nature 502:693-697 (2013); Lin, P., et al. Proc. Nat. Acad. Sci. U.S.A. 115:3538-3546 (2018); Schuchmann, K., Muller, V. Appl. Environ. Microbiol. 82:4056-4069 (2016); Wang, Q., et al. Metab. Eng. 51: 79-87 (2019); Mainguet, S., et al. Metab. Eng. 19:116-127 (2013); Yu, H., et al. Nat. Commun. 9:10 (2018); Francois, J., et al. Front. Bioeng. Biotechnol. 7:16 (2020)). These approaches require major modifications to the host's metabolic network and extensive engineering to circumvent native regulation, limiting the ability to exploit high glycolytic fluxes and generate products efficiently.

Opposed to modifying native metabolism, we propose a new conceptual framework in which orthogonal, new-to-nature carbon and energy conversion pathways facilitate the synthesis of fuels and chemicals from carboxylic acid intermediates (CAis) driven by externally supplied electron donors. This allows the CAi platform to generate diverse products at ≥100% carbon yield while retaining the established high product and energy efficiencies of fermentative metabolism. Furthermore, the CAi platform exploits enzymes and pathways ubiquitous in nature thus enabling its implementation in industrial organisms.

### SUMMARY

As disclosed herein, the carboxylic acid intermediate (CAi) platform is a novel conceptual framework in which new-to-nature carbon conversion and energy generation pathways support the synthesis of diverse reduced products at ≥100% carbon yield. Opposed to current approaches to carbon optimize fermentations which rely on rewiring central metabolism, the CAi platform leverages the orthogonality of these pathways to the host metabolism. This allows retaining the established high product fluxes and energy efficiencies of fermentative metabolism while driving downstream product synthesis from carbon-conserved CAis via externally supplied reducing equivalents. CAis, such as lactic acid, generated at 100% carbon-efficiency link upstream pathways with product synthesis pathways based on novel biochemistries to facilitate the production of a range of products in a carbon-conserving or carbon-consuming manner.

Initiated by activation of the CAi(s), such as converting lactic acid to lactoyl-CoA, the downstream pathways are based on various novel biochemistries, including direct reduction reactions for product synthesis or generation of molecules as precursors for one-carbon (C1) additions via acyloin condensation. The energy required to drive these reactions is derived from reduced C1 compounds via a synthetic C1 bio-reforming pathway that enables generation of NADH and ATP (via substrate level phosphorylation) or H2 for NADH generation via soluble hydrogenases. The ubiquity of fermentative metabolism and the orthogonality of the CAi carbon and energy conversion pathways facilitate implementation in various industrial hosts to develop carbon optimized fermentation systems that avoid carbon loss during reduced product synthesis.

Therefore, disclosed herein are microorganisms that are not naturally able to produce reduced products from carboxylic acid intermediates without carbon loss, but which have been engineered to be able to do so. Engineering of these organisms involves providing a cell system a first set of metabolic enzymes to activate a CA intermediate(s) to the corresponding acyl-CoA, a second set of metabolic enzymes convert the acyl-CoA intermediate to a product, a third set of metabolic enzymes to generate reducing equivalents (e.g. NADH) and ATP from an externally supplied energy source (e.g. reduced one-carbon compounds and/or H2), and feeding the system a CA intermediate or a carbon source that can be converted to a CA intermediate (e.g. glucose) and an externally supplied energy source under suitable conditions for the metabolic enzymes to produce a product more reduced than the initial CA intermediate.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1. Enzymatic pathway for energy (redox and ATP) and formyl-CoA generation required for the carboxylic acid platform.

FIG. 2A-2D. Formyl-CoA generation from formate. A) Pathway of screening FAEs platform for formyl-CoA generation from formate with formaldehyde as a co-substrate; B) Screening results of FAEs candidates with formaldehyde and formate using resting cells; C) Pathway of screening FAEs platform for formyl-CoA generation from formate with acetone as a co-substrate; D) Screening results of FAEs candidates using acetone and formate using growing cultures.

FIG. 3A-3B. Generation of formyl-CoA from formaldehyde using acyl-CoA reductases. Synthesis of glycolyl-CoA-derived glycolate through condensation of formaldehyde and formyl-CoA was used as proxy for pathway flux. (a) Pathway showing flux from formaldehyde to glycolate while ACR can serve as both ACR1 and ACR2. (b) Screening results of various ACR1/ACR2 candidates.

FIG. 4A-4B. Generation of formyl-CoA from methanol derived formaldehyde using different methanol dehydrogenases (MDH) in conjunction with LmACR. Synthesis of glycolyl-CoA-derived glycolate through condensation of formaldehyde and formyl-CoA was used as proxy for pathway flux. (a) Full pathway showing energy (redox) generation (in the form of NADH) and formyl-CoA, which then is fed to glycolate synthesis. Enzymes in bold are overexpressed for experiments in panels (b) and (c). (b) Resting-cell bioconversion of methanol to glycolate using various candidates of MDH. (c) Growing-cell bioconversion of methanol to energy (represented by cell biomass) and formyl-CoA (represented by glycolate production).

FIG. 5. Carboxylic acid platform using formate as the starting carboxylic acid.

FIG. 6. Carboxylic acid platform using formaldehyde as the intermediate aldehyde for the C1 elongation.

FIG. 7A-7B. Conversion of formate to ethylene glycol. A) Pathway for conversion of formate to ethylene glycol; B) in vitro conversion of formate to ethylene glycol using purified enzymes.

FIG. 8A-8D. (a) High throughput resting-cell bioconversion platform for screening C1 elongation enzymes (HACS) and formate activation enzyme (FAE; AbfT in this example) (b) Glycolate production from co-feeding formaldehyde (5 mM) and formate (20 mM) (c) Expression of HACS and FAE (AbfT) are controlled independently by inducible promoters (IPTG and cumate) (d) HACS screening results for C1-C1 condensation reaction represented by glycolate productivity.

FIG. 9A-9B. Cell-free pathway prototyping of the full α-reduction pathway starting from formaldehyde as a proxy. (a) Full iteration to C2 aldehyde, acetaldehyde via α-reduction and associated enzymes used for the cell-free prototyping. (b) Change in the substrate and product concentrations over time with different combination of enzymes are added to the reaction mixture.

FIG. 10A-10B. (a) Synthesis of glycine from glycolate as a proxy for C1-C1 condensation followed by CoA hydrolysis. E. coli strain unable to synthesize glycine was constructed. This strain can only grow either when glycine is supplemented or alternative substrate (glycolate) with appropriate enzymes to synthesize glycine is provided. (b) Strain is able to grow only with glycolate supplementation showing higher growth rate with increasing glycolate concentrations.

FIG. 11. Carboxylic acid platform using formamide as the intermediate (substituted) aldehyde for the C1 elongation.

FIG. 12. Formamidase (MmFmdA) under the control of T7lac promoter in the expression vector (pCDFDuet-1).

FIG. 13. Carboxylic acid platform using C2+ carboxylic acids as the substrate.

FIG. 14A-4B. (a) Carboxylic acid platform using acetic acid as the substrate.

(b) HACS screening results for acetaldehyde-C1 condensation reaction represented by lactic acid productivity.

FIG. 15A-15B. (a) High throughput resting-cell bioconversion platform for screening C1 elongation enzymes (HACS) (b) Expression of HACS and FAE (AbfT) are controlled independently by inducible promoters (IPTG and cumate).

FIG. 16A-16B. (a) Carboxylic acid platform using propionic acid as the substrate.

(b) HACS screening results for propionaldehyde-C1 condensation reaction represented by 2-hydroxybutyric acid productivity

FIG. 17. Carboxylic acid platform using butyric acid as the substrate.

FIG. 18A-18B. (a) Carboxylic acid platform using glycolic acid as the substrate.

(b) HACS screening results for glycolaldehyde-C1 condensation reaction represented by glyceric acid productivity

FIG. 19. Carboxylic acid platform using lactic acid as the substrate.

FIG. 20. Carboxylic acid platform using glyceric acid as the substrate.

FIG. 21. Carboxylic acid platform using 3-hydroxypropionic acid as the substrate.

FIG. 22A-22B. (a) Carboxylic acid platform using oxalic acid as the substrate.

(b) HACS screening results for glyoxylic acid-C1 condensation reaction represented by tartronic acid productivity.

FIG. 23. Carboxylic acid platform using malonic acid as the substrate.

FIG. 24. Carboxylic acid platform using succinic acid as the substrate.

FIG. 25. Carboxylic acid platform using isobutyric acid as the substrate.

FIG. 26. Carboxylic acid platform using isovaleric acid as the substrate.

FIG. 27 Production of 2-hydroxyacid, 3-hydroxyacid, alcohol, 1,2-diol and α,β-unsaturated acid from condensation of carboxylic acid-derived ketones and formyl-CoA.

FIG. 28 Production of 2-hydroxyisobutyric acid, 3-hydroxyisobutyric acid, isobutanol, isobutene glycol and methacrylic acid from condensation of lactic acid-derived acetone and formyl-CoA.

FIG. 29 Production of 2-hydroxy-2-methylbutanoic acid, 3-hydroxy-2-methylbutanoic acid, 2-methylbutan-1-ol, 2-methylbutane-1,2-diol and 2-methylbut-2-enoic acid from condensation of 2-hydroxybutanoic acid-derived butanone and formyl-CoA.

FIG. 30 Production of 2-hydroxy-2-methylpentanoic acid, 3-hydroxy-2-methylpentanoic acid, 2-methylpentan-1-ol, 2-methylpentane-1,2-diol and 2-methylpen-2-enoic acid from condensation of 2-hydroxypentanoic acid-derived pentanone and formyl-CoA.

FIG. 31 Production of 2-hydroxy-2-methylheptanoic acid, 3-hydroxy-2-methylheptanoic acid, 2-methylheptan-1-ol, 2-methylheptane-1,2-diol and 2-methylhept-2-enoic acid from condensation of 2-hydroxyheptanoic acid-derived heptanone and formyl-CoA.

FIG. 32 Production of 2,3-hydroxy-2-methylproptanoic acid, 2-methylpropane-1,3-diol, 2-methylpropane-1,2,3-triol and 3-hydroxy-2-methylacrylic acid from condensation of 2,3-hydroxypropanoic acid-derived hydroxyacetone and formyl-CoA.

FIG. 33 Production of 2-hydroxy-2,3-dimethylbutanoic acid, 3-hydroxy-2,3-dimethylbutanoic acid, 2,3-dimethylbutan-1-ol, 2,3-dimethylbutane-1,2-diol and 2,3-dimethylbut-2-enoic acid from condensation of 2-hydroxy-3methylbutanoic acid-derived 3-methyl-2-butanone and formyl-CoA.

FIG. 34 Production of 2-hydroxy-2-methyl-3-oxopropanoic acid, 2-methylpropane-1,3-diol, and 2-methylheptane-1,2,3-triol from condensation of 2-hydroxy-3-oxopropanoic acid-derived methylglyoxal and formyl-CoA.

FIG. 35 Production of 2-hydroxy-2-methyl-4-oxopentanoic acid, 3-hydroxy-2-methyl-4-oxopentanoic acid, 5-hydroxy-4-methylpentan-2-one, 4,5-dihydroxy-4-methylpentan-2-one and 2-methyl-4-oxopent-2-enoic acid from condensation of 2-hydroxy-4-oxopentanoic acid-derived pentane-2,4-dione and formyl-CoA.

FIG. 36A-36B Methyl ketones as substrate for condensation with formyl-CoA using purified enzymes. A) Pathway for condensation of methyl ketones and formyl-CoA from formate to 2-hydroxy-2-methyl acid; B) GC-MS results of in vitro assays with different methyl ketones and formate. The desired product was pointed with blue arrow.

FIG. 37A-37B. Acetone as substrate for condensation with formyl-CoA using growing cultures. A) Pathway for condensation of acetone and formyl-CoA from formate to 2HIB; B) Production of 2HIB from acetone and formation in E. coli.

FIG. 38 illustrates the generalized carboxylic acid (CA) platform for reduced product synthesis without carbon loss. A carboxylic acid is activated to the corresponding acyl-CoA which can be reduced to various products, many of which can also serve as intermediates for one-carbon (C1) elongation. Reducing equivalents and ATP required for product synthesis are generated via reduced C1 compounds or H2. Exemplary products are shown and represent only a subset of potential products.

FIG. 39 illustrates the generalized carboxylic acid (CA) platform for reduced product synthesis without carbon loss when biomass derived sugars are used as the carbon source. A carboxylic acid is generated from the supplied sugar(s) and then activated to the corresponding acyl-CoA which can be reduced to various products, many of which can also serve as intermediates for one-carbon elongation. Reducing equivalents and ATP required for product synthesis are generated via reduced C1 compounds or H2. Exemplary products are shown and represent only a subset of potential products.

FIG. 40 shows activation, product synthesis, and energy generation pathways for producing reduced products from a supplied carboxylic acid.

FIG. 41 shows activation, product synthesis, and energy generation pathways for producing reduced products from a supplied 2-hydroxycarboxylic acid.

FIG. 42 illustrates formyl-CoA elongation pathways for one-carbon elongation. Reduced one-carbon substrates, such as methanol, are activated to formyl-CoA, the C1 elongation unit through various redox reactions. Formyl-CoA serves to elongate a carbonyl containing compound in a reaction catalyzed by HACL, resulting in the production of 2-hydroxyacyl-CoA. 2-Hydroxyacyl-CoA can be further reduced to a 2-hydroxyaldehyde. 2-hydroxyaldehyde can be reduced to 1,2-diol and dehydrated to an aldehyde. The various intermediates of these elongation pathways can be converted to desirable chemical products including 2-hydroxy-acids, diols, polyols, and alcohols.

Abbreviations: MDH: methanol dehydrogenase; ACR: acyl-CoA reductase; HACL: 2-hydroxyacyl-CoA lyase; ADH: alcohol dehydrogenase; DDR: diol dehydratase; TES: thioesterase. Examples of compounds generated from carboxylic acid activation and reduction (acetone, propionaldehyde, lactaldehyde) that serve as intermediates for C1 elongation are shown. Products that can be derived from these example compounds are also shown with concentrations produced shown.

FIG. 43 illustrates energy generating pathways for reducing equivalent (NADH) and ATP synthesis required to drive product synthesis pathways. Routes from reduced one-carbon compounds (e.g. methanol) and H2 are shown.

FIG. 44 shows specific example of lactic generation from sugars as the carboxylic acid intermediate with activation, product synthesis, and energy generating pathways. Exemplary products are shown and represent only a subset of potential products.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure.

Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As defined herein, the phrases “recombinant host microorganism”, “genetically engineered host microorganism”, “engineered host microorganism” and “genetically modified host microorganism” may be used interchangeably and refer to host microorganisms that have been genetically modified to (a) express one or more exogenous polynucleotides, (b) over-express one or more endogenous and/or one or more exogenous polynucleotides, such as those included in a vector, or which have an alteration in expression of an endogenous gene or (c) knock-out or down-regulate an endogenous gene. In addition, certain genes may be physically removed from the genome (e.g., knock-outs) or they may be engineered to have reduced, altered or enhanced activity.

The terms “engineer”, “genetically engineer” or “genetically modify” refer to any manipulation of a microorganism that results in a detectable change in the microorganism, wherein the manipulation includes, but is not limited to, introducing non-native metabolic functionality via heterologous (exogenous) polynucleotides or removing native-functionality via polynucleotide deletions, mutations or knock-outs. The term “metabolically engineered” generally involves rational pathway design and assembly of biosynthetic genes (or ORFs), genes associated with operons, and control elements of such polynucleotides, for the production of a desired metabolite. “Metabolically engineered” may further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway.

The phrases “metabolically engineered microorganism” and “modified microorganism” are used interchangeably herein, and refer not only to the particular subject host cell, but to the progeny or potential progeny of such a cell. Because certain modifications may occur in succeeding generations due to either mutation or environmental influences, such progeny may not, in fact, be identical to the parent cell, but are still included within the scope of the term as used herein.

The term “mutation” as used herein indicates any modification of a nucleic acid and/or polypeptide which results in an altered nucleic acid or polypeptide (i.e., relative to the wild-type nucleic acid or polypeptide sequence). Mutations include, for example, point mutations, substitutions, deletions, or insertions of single or multiple residues in a polynucleotide (or the encoded polypeptide), which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, an insertion, or a deletion of part or all of a gene. In certain embodiments, a portion of a genetically modified microorganism's genome may be replaced with one or more heterologous (exogenous) polynucleotides. In some embodiments, the mutations are naturally-occurring. In other embodiments, the mutations are the results of artificial selection pressure. In still other embodiments, the mutations in the microorganism genome are the result of genetic engineering.

The term “expression” or “expressed” with respect to a gene sequence, an ORF sequence or polynucleotide sequence, refers to transcription of the gene, ORF or polynucleotide and, as appropriate, translation of the resulting mRNA transcript to a protein. Thus, as will be clear from the context, expression of a protein results from transcription and translation of the open reading frame sequence. The level of expression of a desired product in a host microorganism may be determined on the basis of either the amount of corresponding mRNA that is present in the host, or the amount of the desired product encoded by the selected sequence. For example, mRNA transcribed from a selected sequence can be quantitated by PCR or by northern hybridization (see Sambrook et al., Molecular Cloning: A Laboratory Manual, Cold Spring Harbor Laboratory Press, 1989). Protein encoded by a selected sequence can be quantitated by various methods (e.g., by ELISA, by assaying for the biological activity of the protein, or by employing assays that are independent of such activity, such as western blotting or radioimmunoassay, using antibodies that are recognize and bind reacting the protein).

The term “endogenous”, as used herein with reference to polynucleotides (and the polypeptides encoded therein), indicates polynucleotides and polypeptides that are expressed in the organism in which they originated (i.e., they are innate to the organism). In contrast, the terms “heterologous” and “exogenous” are used interchangeably, and as defined herein with reference to polynucleotides (and the polypeptides encoded therein), indicates polynucleotides and polypeptides that are expressed in an organism other than the organism from which they (i.e., the polynucleotide or polypeptide sequences) originated or where derived.

The term “feedstock” is defined as a raw material or mixture of raw materials supplied to a microorganism, or fermentation process, from which other products can be made. For example, as set forth in the present invention, a glucose carbon source or a xylose carbon source, either alone or in combination, are biomass-derived feedstocks for a microorganism that produces a bio-fuel or bio-based chemical in a fermentation process. However, in addition to a feedstock (e.g., a glucose substrate) of the invention, the fermentation media contains suitable minerals, salts, cofactors, buffers and other components, known to those skilled in the art, suitable for the growth of the cultures and promotion of the enzymatic pathways necessary for multi-carbon compound production.

The term “substrate” refers to any substance or compound that is converted, or meant to be converted, into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material (e.g., methane), but also intermediate and end product metabolites used in a pathway associated with a metabolically engineered microorganism as described herein.

The term “fermentation” or “fermentation process” is defined as a process in which a host microorganism is cultivated in a culture medium containing raw materials, such as feedstock and nutrients, wherein the microorganism converts raw materials, such as a feedstock, into products.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, including DNA, RNA, ORFs, analogs and fragments thereof.

As defined herein, the term “open reading frame” (hereinafter, “ORF”) means a nucleic acid or nucleic acid sequence (whether naturally occurring, non-naturally occurring, or synthetic) comprising an uninterrupted reading frame consisting of
(i) an initiation codon, (ii) a series of two (2) of more codons representing amino acids, and (iii) a termination codon, the ORF being read (or translated) in the 5′ to 3′ direction.

It is understood that the polynucleotides described herein include “genes” and that the nucleic acid molecules described herein include “vectors” or “plasmids”.

Accordingly, the term “gene”, refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “promoter” refers to a nucleic acid sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “codon-optimized” as it refers to genes or coding regions of nucleic acid molecules (or ORFs) for transformation of various hosts, refers to the alteration of codons in the gene or coding regions of the nucleic acid molecules to reflect the typical codon usage of the host organism without altering the polypeptide encoded by the DNA.

The term “operon” refers to two or more genes which are transcribed as a single transcriptional unit from a common promoter. In certain embodiments, the genes, polynucleotides or ORFs comprising the operon are contiguous genes. It is understood that transcription of an entire operon can be modified (i.e., increased, decreased, or eliminated) by modifying the common promoter. Alternatively, any gene, polynucleotide or ORF, or any combination thereof in an operon can be modified to alter the function or activity of the encoded polypeptide. The modification can result in an increase or a decrease in the activity or function of the encoded polypeptide. Further, the modification can impart new activities on the encoded polypeptide.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes”, that is, that replicate autonomously or can integrate into a chromosome of a host microorganism. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

The term “homolog”, as used with respect to an original enzyme, polypeptide, gene or polynucleotide (or ORF encoding the same) of a first family or species, refers to distinct enzymes, genes or polynucleotides of a second family or species, which are determined by functional, structural or genomic analyses to be an enzyme, gene or polynucleotide of the second family or species, which corresponds to the original enzyme or gene of the first family or species. Most often, “homologs” will have functional, structural or genomic similarities. Techniques are known by which homologs of an enzyme, gene or polynucleotide can readily be cloned using genetic probes and PCR. Identity of cloned sequences as “homologs” can be confirmed using functional assays and/or by genomic mapping of the genes.

A polypeptide (or protein or enzyme) has “homology” or is “homologous” to a second polypeptide if the nucleic acid sequence that encodes the polypeptide has a similar sequence to the nucleic acid sequence that encodes the second polypeptide.

Alternatively, a polypeptide has homology to a second polypeptide if the two proteins have “similar” amino acid sequences. Thus, the terms “homologous proteins” or “homologous polypeptides” is defined to mean that the two polypeptides have similar amino acid sequences. In certain embodiments of the invention, polynucleotides and polypeptides homologous to one or more polynucleotides and/or polypeptides set forth in Table 1 may be readily identified using methods known in the art for sequence analysis and comparison.

A homologous polynucleotide or polypeptide sequence of the invention may also be determined or identified by BLAST analysis (Basic Local Alignment Search Tool) or similar bioinformatic tools, which compare a query nucleotide or polypeptide sequence to a database of known sequences. For example, a search analysis may be done using BLAST to determine sequence identity or similarity to previously published sequences, and if the sequence has not yet been published, can give relevant insight into the function of the DNA or protein sequence.

In various embodiments, the invention provides for a carboxylic acid (CA) compound, either directly or generated from a compound such as biomass-derived sugar(s), serving as the source of carbon for the organism. This CA intermediate is activated to the corresponding acyl-CoA intermediate, with multiple C-conserving or C-fixing downstream product synthesis pathways engineered to generate diverse reduced products. For energy generation required to drive these reactions and avoid CO2 evolution, compounds such as reduced one-carbon (C1) molecules (e.g. methanol) or hydrogen (H2) serve as the energy source for the organism, with synthetic routes to utilize these compounds to generate NADH and ATP. Together, these enable the disclosed system to produce reduced compounds without carbon loss.

In some embodiments, carboxylic acid (CA) molecules are the solely supplied carbon source. In some embodiments, a sugar such as glucose can be solely supplied carbon source and is converted to the carboxylic acid (CA) molecules prior to activation. In these situations, the A3 molecule is first activated to the corresponding acyl-CoA by a suitable acyl-CoA synthetase, or an acyl-CoA transferase, or a carboxylate kinase and a phosphotransacylase, or a carboxylic acid reductase and an acyl-CoA reductase, or an aldehyde dehydrogenase and an acyl-CoA reductase.

The acyl-CoA initiates product synthesis pathways and enables additional biochemical reactions to generate a range of products as well as target intermediates amenable to elongation. For example, a C1 unit (formyl-CoA) can be added to aldehydes and ketones through 2-hydroxyacyl-CoA lyases. These reactions and combinations thereof result in intermediates amenable to further biochemical reactions that significantly expand the range of attainable products.

For energy generation required to drive these reactions and avoid CO2 evolution, reducing equivalents in the form of NAD(P)H and ATP are generated from an externally supplied energy source. In some embodiments, reduced one-carbon (C1) molecules, such as methanol, are supplied as the energy source. In these situations, the C1 molecule is oxidized to CO2 by suitable enzymes with concomitant generation of NADH. For example, methanol can be oxidized to CO2 through a methanol dehydrogenase converting methanol to formaldehyde, a formaldehyde dehydrogenase converting formaldehyde to formate, and a formate dehydrogenase converting formate to CO2. In some embodiments, the reduced one-carbon (C1) is oxidized using suitable enzymes that generate both NAD(P)H and ATP. For example, methanol can be oxidized to CO2 through a methanol dehydrogenase converting methanol to formaldehyde, an acylating formaldehyde dehydrogenase converting formaldehyde to formyl-CoA, a phosphate formyltransferase converting formyl-CoA to formyl-phosphate, a formate kinase converting formyl-phosphate to formate, and a formate dehydrogenase converting formate to CO2. In some embodiments, hydrogen (H2) is supplied as the energy source. In these situations, H2 is converted to NAD(P)H through suitable NAD⁺-reducing hydrogenases.

Therefore, disclosed herein is a method for enabling the production of reduced products from a CA intermediate without carbon loss comprising a cell system containing a first set of metabolic enzymes to activate a CA intermediate to the corresponding acyl-CoA intermediate, a second set of metabolic enzymes to convert said acyl-CoA intermediate to a product, a third set of metabolic enzymes to generate reducing equivalents and ATP from an externally supplied energy source, and feeding the system the CA intermediate, or a carbon source from which the CA intermediate can be generated, and an external energy source under suitable conditions for the metabolic enzymes to produce the desired reduced compounds.

The first step in the disclosed systems and methods is the conversion of the CA intermediate (e.g. lactic acid) into the corresponding acyl-CoA (e.g. lactoyl-CoA). This step is referred to herein as CA activation. In general, CA activation involves the conversion of a carboxylic acid group to a CoA thioester and requires at least one of following steps: (1) the carboxylic acid is directly converted to the corresponding acyl-CoA by an acyl-CoA synthetase or an acyl-CoA transferase, (2) the carboxylic acid is converted to a phosphate intermediate by a carboxylate kinase which is then converted to the corresponding acyl-CoA by a phosphotransacylase, (3) the carboxylic acid is first reduced to the corresponding aldehyde by a carboxylic acid reductase which is then converted to the corresponding acyl-CoA by an acyl-CoA reductase, or (4) the carboxylic acid is first reduced to the corresponding aldehyde by an aldehyde dehydrogenase which is then converted to the corresponding acyl-CoA by an acyl-CoA reductase.

The second step in the disclosed systems and methods is the conversion of the acyl-CoA into a desired reduced product(s) This step is referred to herein as product synthesis. In general, diverse reduced products with varying functionality and chain length can be synthesized from the acyl-CoA intermediate. For example, reduction of the acyl-CoA by an acyl-CoA reductase can generate an aldehyde. This aldehyde can be further reduced to an n-alcohol by an alcohol dehydrogenase or serve as a precursor for formyl-CoA elongation using formyl-CoA as the C1 building block in a reaction catalyzed by a 2-hydroxyacyl-CoA lyase (HACL) or an oxalyl-CoA decarboxylase (OXC). These enzymes can ligate formyl-CoA with a variety of carbonyl-containing acceptors of broad chain length and functionalization, including aldehydes and ketones.

In some embodiments, the initial carboxylic acid and corresponding acyl-CoA contains a hydroxy (—OH) group at the second carbon (2-hydroxyacyl-CoA). In these situations, reduction of the CoA group by and an acyl-CoA reductase and an alcohol dehydrogenase results in the formation of a 1,2-diol which can be further dehydrated by a diol dehydratase to the corresponding ketone or aldehyde. This ketone or aldehyde can also serve as a precursor for formyl-CoA elongation using formyl-CoA as the C1 building block.

In some embodiments, the 2-hydroxyacyl-CoA formed through the addition of formyl-CoA to an aldehyde or ketone is reduced to a 2-hydroxyaldehyde by an acyl-CoA reductase (ACR; E.C. 1.2.1.-, e.g. 1.2.1.10, 1.2.1.76, 1.2.1.84). Further reduction of the 2-hydroxyaldehyde to give a 1,2-diol is possible by a suitable 1,2-diol oxidoreductase (DOR; E.C. 1.1.1.77) or alcohol dehydrogenase (ADH; E.C. 1.1.1.71). Dehydration of the 1,2-diol can be catalyzed by the activity of diol dehydratase (DDR; E.C. 4.2.1.28) to give an aldehyde, which can be further reduced to an alcohol by an alcohol dehydrogenase (ADH; E.C. 1.1.1.71).

In some embodiments, a combination of the above routes can be implemented at the same time such that for some molecules, products of the same chain length as the acyl-CoA are formed, whereas for other molecules, elongation takes place. Both routes can be simultaneously present at the same time in the same system.

In some embodiments, the products of interest are the products of the above reactions. Examples of these products include, but are not limited to alcohols, such as ethanol, propanol, and butanol; diols, such as 1,2-propanediol, 1,2-butanediol, and 1,3-butanediol; triols, such as 1,2,3-butanetriol; hydroxy carboxylic acids, such as lactate, 2-hydroxybutyrate, and 2,3-dihydroxybutyrate; and ketones, such as acetone.

The third step in the disclosed systems and methods is the generation of reducing equivalents and ATP from an external energy source. This step is referred to herein as energy generation. In general, energy generation involves the oxidation of a supplied external energy source resulting in the generation of reducing equivalents and ATP. In some embodiments, reduced one-carbon (C1) molecules, such as methanol, are supplied as the energy source. In these situations, the C1 molecule is oxidized to CO2 by suitable enzymes with concomitant generation of NADH. For example, methanol can be oxidized to CO2 through a methanol dehydrogenase converting methanol to formaldehyde, a formaldehyde dehydrogenase converting formaldehyde to formate, and a formate dehydrogenase converting formate to CO2. In some embodiments, the reduced one-carbon (C1) is oxidized using suitable enzymes that generate both NAD(P)H and ATP. For example, methanol can be oxidized to CO2 through a methanol dehydrogenase converting methanol to formaldehyde, an acylating formaldehyde dehydrogenase converting formaldehyde to formyl-CoA, a phosphate formyltransferase converting formyl-CoA to formyl-phosphate, a formate kinase converting formyl-phosphate to formate, and a formate dehydrogenase converting formate to CO2. In some embodiments, hydrogen (H2) is supplied as the energy source. In these situations, H2 is converted to NAD(P)H through suitable NAD⁺-reducing hydrogenases.

In some embodiments, the described pathways are provided within the context of a microbial host. In some embodiments, the microbial host is cultured in a fermentation system to produce desired products. In other embodiments, a microbial system is used to produce the enzymes, which are then extracted from the microbes for use in a cell-free system. In other embodiments, the enzymes are produced separately and individually added to the system.

The pathway in a living system is generally made by transforming the microbe with one or more expression vector(s) containing a gene encoding one or more of the enzymes, but the genes can also be added to the chromosome by recombinant engineering, homologous recombination, gene editing, and similar techniques. Where the needed protein is endogenous, as is the case in some instances, it may suffice as is, but is usually overexpressed for better functionality and control over the level of active enzyme. In some embodiments, one or more, or all, such genes are under the control of an inducible promoter.

The enzymes can be added to the genome or via expression vectors, as desired. Preferably, multiple enzymes are expressed in one vector or multiple enzymes can be combined into one operon by adding the needed signals between coding regions. Further improvements can be had by overexpressing one or more, or even all of the enzymes, e.g., by adding extra copies to the cell via plasmid or other vector. Initial experiments may employ expression plasmids hosting 3 or more ORFs for convenience, but it may be preferred to insert operons or individual genes into the genome for stability reasons.

Still further improvements in yield can be had by reducing competing pathways, such as those pathways for making e.g., acetate, formate, ethanol, and lactate, and it is already well known in the art how to reduce or knockout these pathways. Sec e.g., U.S. Pat. Nos. 7,569,380, 7,262,046, 8,962,272, 8,795,991, 8,129,157, and 8,691,552, each incorporated by reference herein in its entirety for all purposes. Many others have worked in this area as well.

Following the construction of a suitable strain containing the engineered pathway, culturing of the developed strains can be performed to evaluate the effectiveness of the pathway at its intended goal—the production of products more reduced than the carboxylic acid intermediate. The organism can be cultured in a suitable growth medium, and can be evaluated for product formation from carboxylic acid substrates, where the carboxylic acid is either supplied as the carbon source or generated from a different supplied carbon source (e.g. glucose). The amount of products produced by the organism can be measured by UPLC or GC, and indicators of performance such as growth rate, productivity, titer, yield, or carbon efficiency can be determined.

Further evaluation of the interaction of the pathway enzymes with each other and with the host system can allow for the optimization of pathway performance and minimization of deleterious effects. Because the pathway is under synthetic control, rather than under the organism's natively evolved regulatory mechanisms, the expression of the pathway is usually manually tuned to avoid potential issues that slow cell growth or production and to optimize production of desired compounds.

Additionally, an imbalance in relative enzyme activities might restrict overall carbon flux throughout the pathway, leading to suboptimal production rates and the buildup of pathway intermediates, which can inhibit pathway enzymes or be cytotoxic. Analysis of the cell cultures by HPLC or GC can reveal the metabolic intermediates produced by the constructed strains. This information can point to potential pathway issues.

As an alternative to the in vivo expression of the pathway, a cell free in vitro version of the pathway can be constructed. By purifying the relevant enzyme for each reaction step, the overall pathway can be assembled by combining the necessary enzymes in a reaction mixture. With the addition of the relevant cofactors and substrates, the pathway can be assessed for its performance independently of a host.

General methods for gene synthesis and DNA cloning, as well as vector and plasmid construction, are well known in the art, and are described in a number of publications. More specifically, techniques such as digestion and ligation-based cloning, as well as in vitro and in vivo recombination methods, can be used to assemble DNA fragments encoding a polypeptide that catalyzes a substrate to product conversion into a suitable vector. These methods include restriction digest cloning, sequence- and ligation-independent Cloning (SLIC), Golden Gate cloning, Gibson assembly, and the like. Some of these methods can be automated and miniaturized for high-throughput applications.

Gene cassettes for expressing an engineered metabolic pathway in a host microorganism are known in the art. The cassette can comprise one or more open reading frames (ORFs) which encode the enzymes of the introduced pathway, a promoter for directing transcription of the downstream ORF(s) within the operon, ribosome binding sites for directing translation of the mRNAs encoded by the individual ORF(s), and a transcriptional terminator sequence. Due to the modular nature of the various components of the expression cassette, one can create combinatorial permutations of these arrangements by substituting different components at one or more of the positions. One can also reverse the orientation of one or more of the ORFs to determine whether any of these alternate orientations improve the product yield.

In some embodiments, the host microorganism for expressing metabolic pathway genes contains plasmid vector(s) with the metabolic pathway expression cassettes mobilized into these organisms via conjugation.

In an alternative method for expressing metabolic pathway genes in a microbial host, the biosynthetic pathway genes can be inserted directly into the chromosome. Methods for chromosomal modification include both non-targeted and targeted deletions and insertions.

In some embodiments, the disclosed systems and methods also involve recovering and purifying the desired product from the fermentation broth. The method to be used depends on the physico-chemical properties of the product and the nature and composition of the fermentation medium and cells. For example, U.S. Pat. No. 8,101,808 describes methods for recovering C3-C6 alcohols from fermentation broth using continuous flash evaporation and phase separation processing. In some embodiments, solids may be removed from the fermentation medium by centrifugation, filtration, decantation. In some embodiments, the multi-carbon compounds are isolated from the fermentation medium using methods such as distillation, azeotropic distillation, liquid-liquid extraction, adsorption, gas stripping, membrane evaporation, or pervaporation.

Disclosed herein are several exemplary, non-limiting embodiments of the system, methods and compositions disclosed herein. In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

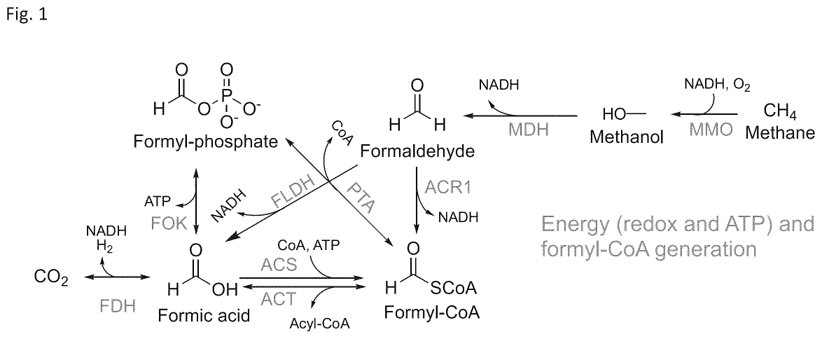

For example, in some embodiments, the microorganisms are modified to express one or more enzymes responsible for the catalytic reactions as depicted in FIG. 1, 2, 3, 4, 5, 6, 7, 8, 9, 11, 13, 14, 16, 17, 18, 19, 20, 21, 22, 23, 24, 25, 26, 27, 28, 29, 30, 31, 32, 33, 34, 35, 36, 37. Suitably, the microorganisms are modified to express two or more enzymes, three or more enzymes, four or more enzymes, five or more enzymes, and can be determined from the pathways depicted in the figures and not to be limited by the examples described herein. Further, one skilled in the art would be able to pick the combination of enzymes to be introduced into the microorganism to produce a genetically modified organism capable of modifying a carbon source to the desired chemical outcome.

In some embodiments, microorganisms are modified with enzymes responsible for energy, redox and ATP, and formyl-CoA generation. In any and all combination, the microorganism is modified to include one or more of the following enzymes, including, for example, at least one of, mmoXyBZCD, pmoA1A2B1B2, BmMDH2, CnMCH2, BsMDH, LmACR, StEutE, ChAld, EcMhpF, PsDmpF, EcFrmA, PpFdhA, CcPta-Ack, EcACS, StACSstab, MhACS, ArACS, CaAbft, OfFrc, PsFdh and/or CbFdh.

In some embodiments, microorganisms are modified with enzymes responsible for carboxylic acid platform using formaldehyde as the intermediate aldehyde for the C1 elongation. In any and all combinations, the microorganism is modified to include one or more of the following enzymes, including, for example, at least one of, CcPta-A ck, EcPta-Ack, EcACS, StACSstab, MhACS, ArACS, CaAbfT, OfFrc, LmACE, RuHACL, BsmHACL, AcHACK, MeOXC4, LmACR, StPduP, EcAldA, EcfucO, KoPddABC, EcAdhE, CcPta-Ack, EcPta-Ack, EcYciA, EcGlcD, MtAld, BsAld, ALAT1, aldH1, dhaS, EcSerC, GOT1 and/or Ec YdfG.

In some embodiments, microorganisms are modified with enzymes responsible for carboxylic acid platform using formamide as the intermediate aldehyde for the C1 elongation. In any and all combinations, the microorganism is modified to include one or more of the following enzymes, including, for example, at least one of, MmFmdA, RuHACL, BsmHACL, ACHACL, MeOXC4, LmACR, EcAldA, EcfucO, KoPddA BC, EcAdhE, CcPta-Ack, EcPta-Ack, EcYciA, EcGlcD, MtAld, BsAld, ALA TI, aldH1, dhaS, EcSerC, GOT1, EcYdfG and/or EcGldA.

In some embodiments, microorganisms are modified to express enzymes responsible for energy (redox and ATP) and formyl-CoA generation pathways. In any and all combinations, the microorganism is modified to include one or more of the following enzymes, including, for example, at least one of, mmoB, mdh2, adh, frmA, LmACR, mhpF, dmpF, cutE, CcPta-AcK, EcPta-AcK, EcACS, StACSstabm MhACS, ArACS, abfT, frc and/or fdh.

In some embodiments, microorganisms are modified with enzymes responsible for activation-reduction and C1 elongation pathways. In any and all combinations, the microorganism is modified to include one or more of the following enzymes, including, for example, at least one of, ackA, tdcD, buk1, pta, ptb, yfaC, prpE, IvaE, AAE3, sucC, sucD, cat1, scoC, cat3, yfdE, pct, cutE, pduP, adhE2, sucD, RuHACL, MeOXC4, JG115, ydiF, tesA, Idh, ald, pdh, pduP, yahK, pduC, pduD, pduE, pddA, pddB, pddC, PiDD, yahk, lcdAB, acuN, gbuF, acuK, crt, IvaC, acuK, tesB, pcs, bcd, fade, pduP and/or ald.

In some embodiments, microorganisms are modified with enzymes responsible for carboxylic acid platform using methyl ketones for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In some embodiments, microorganisms are modified with enzymes responsible for carboxylic acid platform using acetone for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, ChAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PIECH, CaHbd, EcAldA, CaAbfT, OfFrc, PsFdh, CbFdh and/or EcYciA.

In some embodiments, microorganisms are modified with enzymes responsible for carboxylic acid platform using butanone for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, ChAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PIECH, CaHbd, EcAldA, CaAbfT, OfFrc, PsFdh, CbFdh and/or EcYciA.

In some embodiments, microorganisms are modified with enzymes responsible for carboxylic acid platform using pentanone for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PIECH, CaHbd, EcAldA, CaAbfT, OfFrc, PsFdh, CbFdh and/or Ec YciA.

In some embodiments, microorganisms are modified with enzymes responsible for carboxylic acid platform using heptanone for the C-1 elongation pathway. In any and all combinations, these enzymes include at least one of the microorganism is modified to include one or more of the following enzymes, including, for example, at least one of, mmoXYBDCD, pmoA1A2B1B2, BmMDH2, CnMDH2, BsMDH, LmACR, StEutE, ChAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PIECH, CaHbd, EcAldA, CaAbfT, OfFrc, PsFdh, CbFdh and/or EcYciA.

In some embodiments, microorganisms are modified with enzymes responsible for carboxylic acid platform using hydroxyacetone for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PIECH, CaHbd, EcAldA, CaAbfT, OfFrc, PsFdh, CbFdh and/or EcYciA.

In some embodiments, microorganisms are modified with enzymes responsible for carboxylic acid platform using 3-methyl-2-butanone for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, ChAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PIECH, CaHbd, EcAldA, CaAbfT, OfFrc, PsFdh, CbFdh and/or Ec YciA.

In some embodiments, microorganisms are modified with enzymes responsible for carboxylic acid platform using methylgloxyal for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, ChAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PIECH, CaHbd, EcAldA, CaAbfT, OfFrc, PsFdh, CbFdh and/or Ec YciA.

In some embodiments, microorganisms are modified with enzymes responsible for carboxylic acid platform using acetylacetone (pentane-2, 4-ddione) for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In specific embodiments, microorganisms are modified with enzymes responsible for the generation of formyl-CoA from formate. In any and all combinations, these enzymes include, for example, at least one of AbfT, CcPta-Ack, CaAbfT, OfFrc, EcACS, MhACS, MhACS3, ArACS, StACS6 and/or StACS.

In specific embodiments, microorganisms are modified with enzymes responsible for the generation of formyl-CoA from formaldehyde using acyl-CoA reductase. In any and all combinations, these enzymes include, for example, at least one of LmACR, StEutE, EcmhpF and/or PsDmpF.

In specific embodiments, microorganisms are modified with enzymes responsible for the generation of formyl-CoA from methanol derived formaldeyde using different methanol dehydrogenases in conjunction with LmACR. In any and all combinations, these enzymes include, for example, at least one of BmMDH, CnMdh, RuHACL and/or EcAldA.

In specific embodiments, microorganisms are modified with enzymes responsible for the conversion of formate to ethylene glycol. In any and all combinations, these enzymes include, for example, at least one of LmACR, OfFrc and/or HACS.

In specific embodiments, microorganisms are modified with enzymes responsible for the conversion of glycolate production. In any and all combinations, these enzymes include, for example, at least one of AbfT, HACS, RuHACL, BsmHACL, MeOXC4 and/or AcHACL.

Specific embodiments demonstrate the cell-free method for prototyping C1 elongation pathway for formaldehyde as a starting substrate, which could be generated through activation and reduction of carboxylic acid, formate. In any and all combination, the enzymes may include, LcACR, RuHACL, EcFucO, and/or KoPddABC.

In specific embodiments, microorganisms are modified with enzymes responsible for the synthesis of glycine from glycolate. In any and all combinations, these enzymes include, for example, at least one of AbfT, HACS, RuHACL, BsmHACL, MeOXC4 and/or AcHACL.

In specific embodiments, microorganisms are modified with enzymes responsible the implementation of the carboxylic acid (CA) platform using acetic acid (R═H) as the CA intermediate with C1 elongation pathways. In any and all combination, the enzymes may include, for example, JGI15, RuHACL and/or MeOXC4.

In specific embodiments, microorganisms are modified with enzymes responsible the implementation of the carboxylic acid (CA) platform using propionic acid (R═CH3) as the CA intermediate with C1 elongation pathways. In any and all combination, the enzymes may include, for example, JGI15, RuHACL and/or AcHACL.

In specific embodiments, microorganisms are modified with enzymes responsible the implementation of the carboxylic acid (CA) platform using glycolic acid (R═OH) as the CA intermediate with C1 elongation pathways. In any and all combination, the enzymes may include, for example, JGI15, RuHACL and/or AcHACL.

In specific embodiments, microorganisms are modified with enzymes responsible the implementation of the carboxylic acid (CA) platform using oxalic acid (R═OOH) as the CA intermediate with C1 elongation pathways. In any and all combination, the enzymes may include, for example, JGI15 and/or RuHACL.

In specific embodiments, microorganisms are modified with enzymes responsible the condensation of methyl ketones with formyl-CoA using purified enzymes. In any and all combination, the enzymes may include, for example, CaAbfT and/or BsmHACS.

In specific embodiments, microorganisms are modified with enzymes responsible the implementation of the condensation of methyl ketone with formyl-CoA (generated from formate) in vivo using growing cells, with acetone used as a representative methylketone. In any and all combination, the enzymes may include, for example, BsmHACS and/or AcHCS.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

This Example demonstrates the implementation of the carboxylic acid (CA) platform using lactic acid (lactate) as the CA intermediate. Lactate can be supplied as the carbon source or generated from a carbon source such as glucose and/or other biomass-derived sugars. Conversion of sugars to lactate can leverage the efficiency of fermentative metabolism, namely (L- or D-) lactic acid fermentation, which operates at high titer (>200 g/L), rate (>3 g/L/h), and yield (near theoretical maximum of 1 g/g) and has been demonstrated in numerous microorganisms on an industrial scale (Rawoof, S., et al. Environ. Chem. Lett. 19: 539-556 (2021); Mora-Villalobos, J. A., et al. Fermentation 6:21 (2020); de Oliveira, R. A., et al. Biochem. Eng. J. 133:219-239 (2018)). In this example, established knowledge on high titer, rate, and yield D- and L-lactate production in Escherichia coli (Mazumdar, S., et al. Microb. Cell Fact. 12:7 (2013); Mazumdar, S., et al. Appl. Environ. Microbiol. 76:4327-4336 (2010); Zhu, Y., et al. Appl. Environ. Microbiol. 73:456-464 (2007); Zhao, J. F., et al. Microb. Cell Fact. 12: (2013)) and previous research on engineering Zymomonas mobilis strains for lactate production (Liu, Y., et al. Metab. Eng. 61:261-274 (2020)) are exploited to generate host strains for D- or L-homolactate fermentation from sugars. It is understood to those skilled in the art that other microbial hosts are suitable for direct supply and conversion of carboxylic acids or the generation of carboxylic acids from other carbon sources such as sugars.

Activation of lactate to lactoyl-CoA utilizes an acyl-CoA synthetase from Thermococcus kodakarensis (Awano, T., et al. J. Bacteriol. 196:140-147 (2014)), with subsequent product synthesis pathways engineered to generate products with varying functionality and chain length in the host strain.

Reduction of lactoyl-CoA to lactaldehyde proceeds through the CoA-dependent aldehyde dehydrogenase PduP from Salmonella enterica (Niu, W. and Guo, J., ACS Syn. Biol. 4:378-382 (2015)). The specific lactaldehyde enantiomer, determined by the enantiomer of lactate, is then reduced to the corresponding 1,2-propanediol enantiomer through the action of the lactaldehyde reductase YahK from E. coli (Niu, W. and Guo, J., ACS Syn. Biol. 4:378-382 (2015)). 1,2-PDO is then dehydrated to either propanal or acetone depending on the 1,2-PDO enantiomer and the diol dehydratase utilized. Diol dehydratases such as those from Roseburia inulinivorans (LaMattina, J. W., et al. J. Biol. Chem. 291:15515-15526 (2016)) and Klebsiella oxytoca (Tobimatsu, T., et al. Arch. Biochem. Biophys. 347:132-40 (1997)) are used for this conversion. Propanal is then further reduced to 1-propanol by an alcohol dehydrogenase such as PduQ from Salmonella enterica (Cheng, S., et al. PLOS ONE 7:e47144 (2012)). Alternatively, if acetone is formed from 1,2-PDO dehydration further reduction by an alcohol dehydrogenase such as the secondary alcohol dehydrogenase from Trichomonas vaginalis yields 2-propanol (Sutak, R., et al. FEBS J. 279:2768-2780 (2012)).

To generate the reducing equivalents and ATP required for reduced product synthesis, methanol is supplied as the external energy source. Methanol oxidation proceeds through the NADH-dependent methanol dehydrogenase 2 from Bacillus methanolicus (Roth, T. B., et al. ACS Syn. Biol. 8:796-806 (2019)) to form formaldehyde and generate a NADH. To enable ATP generation directly from the provided C1, a CoA acylating formaldehyde dehydrogenase, phosphate formyltransferase, and formate kinase are used to convert formaldehyde to formate through formyl-CoA and formyl-phosphate intermediates. Here the CoA acylating formaldehyde dehydrogenase from Listeria monocytogenes (Chou, A., et al. Nat. Chem. Biol. 15:900-906 (2019)) and the phosphate formyltransferase and formate kinase enzymes from Clostridium cylindrosporum (Sly, W. S. and Stadtman, E. R. J. Biol. Chem. 238:2639-2647 (1963)) are used to generate an additional NADH and an ATP during this conversion. Formate oxidation to CO2 through the NADH-dependent formate dehydrogenase from Pseudomonas sp. (strain 101) is used to generate another NADH (Tishkov, V. I., et al. Biochem. Biophys. Res. Commun. 192:976-81 (1993)).

The genes for overexpression are either cloned into appropriate vectors or inserted into chromosome with strong synthetic constitutive promoter, such as M1-93. When cloned into vectors, these genes are amplified through PCR using appropriate primers to append homology on each end for recombination into the vector backbone with e.g., Phusion polymerase (Thermo Scientific, Waltham, MA) to serve as the gene insert. Plasmids are linearized by the appropriate restriction enzymes (New England Biolabs, Ipswich, MA, USA) and recombined with the gene inserts using the In-Fusion HD Eco-Dry Cloning system. The mixture is subsequently transformed into Stellar competent cells. Transformants that grow on solid media (LB+Agar) supplemented with the appropriate antibiotic are isolated and screened for the gene insert by PCR. Plasmids from verified transformants are isolated and the sequence of the gene insert is further confirmed by DNA sequencing. The sequence confirmed plasmids are then introduced to host strain through electroporation.

When inserted into chromosome, CRISPR is used and genetic sites of tesB and adhE are suitable loci, although others could be used. The CRISPR method is based on the method developed by Jiang et al. (Jiang, Y., et al. Appl. Environ. Microbiol. 81:2506-2514 (2015)). First, the host strain is transformed with plasmid pCas, the vector for expression of Cas9 and A-red recombinase. The resulting strain is grown under 30° C. with L-arabinose for induction of A-red recombinase expression, and when OD reaches ˜0.6, competent cells are prepared and transformed with pTargetF (AddGene 62226) expressing sgRNA and N20 spacer targeting the locus and template of insertion of target gene. The template is the inserted gene plus M1-93 promoter with ˜500 bp sequences homologous with upstream and downstream of the insertion locus, constructed through overlap PCR with usage of Phusion polymerase or synthesized by GenScript (Piscataway, NJ) or GeneArt® (Life Technologies, Carlsbad, CA). The way to switch N20 spacer of pTargetF plasmid is inverse PCR with the modified N20 sequence hanging at the 5′ end of primers with usage of Phusion polymerase and followed by self-ligation with usage of T4 DNA ligase and T4 polynucleotide kinase (New England Biolabs, Ipswich, MA, USA). Transformants that grow under 30° C. on solid media (LB+Agar) supplemented with spectinomycin and kanamycin (or other suitable antibiotic) are isolated and screened for the chromosomal gene insert by PCR. The sequence of the gene insert, which is amplified from genomic DNA through PCR using Phusion polymerase, is further confirmed by DNA sequencing. The pTargetF can then be cured through IPTG induction, and pCas can be cured through growth under higher temperature like 37-42° C.

All molecular biology techniques are performed with standard methods (Sambrook et al., Molecular Cloning: A Laboratory Manual, Cold Spring Harbor Laboratory Press, 1989).) or by manufacturer protocol. Strains are stored in glycerol stocks at −80° C. Plates are prepared using LB medium containing 1.5% agar, and appropriate antibiotics are included at the following concentrations: ampicillin (100 μg/mL), kanamycin (50 μg/mL), spectinomycin (50 μg/mL) and chloramphenicol (12.5 μg/mL).

MOPS minimal medium (Neidhardt et al. J. Bacteriol. 119736-47 (1974)) with 125 mM MOPS and Na₂HPO₄ in place of K₂HPO₄ (2.8 mM), supplemented with 20 g/L glucose, 10 g/L tryptone, 5 g/L yeast extract, 100 μM FeSO₄, 5 mM (NH₄)₂SO₄, and 30 mM NH₄Cl is used for fermentations. If required, 55 g/L of CaCO3 is also supplemented as pH buffer. 20 mM lactic acid is supplemented, if it is not synthesized intracellularly and needed for the experiment. 500 mM methanol is also supplemented. Antibiotics (50 μg/mL carbenicillin, 50 μg/mL spectinomycin and 50 μg/mL kanamycin) are included when appropriate. All chemicals are obtained from Fisher Scientific Co. (Pittsburgh, PA) and Sigma-Aldrich Co. (St. Louis, MO).

Fermentations are performed in 25 mL Pyrex Erlenmeyer flasks (narrow mouth/heavy duty rim, Corning Inc., Corning, NY) filled with appropriate volume of fermentation medium and sealed with foam plugs filling the necks. For anaerobic conditions, 17.5 mL Hungate tubes are completely filled with fermentation medium and sealed with tubber septa. A single colony of the desired strain is cultivated overnight (14-16 hrs) in LB medium with appropriate antibiotics and used as the inoculum with initial OD₆₀₀ as ˜0.05. After inoculation, flasks are incubated in a NBS 124 Benchtop Incubator Shaker (New Brunswick Scientific Co., Inc., Edison, NJ) at 200 rpm and 37° C. or 30° C. When optical density (550 nm, OD₅₅₀) reached ˜0.3-0.5, appropriate concentration of isopropyl beta-D-1-thiogalactopyranoside (IPTG) (or other suitable inducer) is added for plasmid gene induction. Additional fermentations are conducted in a SixFors multi-fermentation system (Infors HT, Bottmingen, Switzerland) with an air or argon flowrate of 2 N L/hr, independent control of temperature)(37° C., pH (controlled at 7.0 with NaOH and H₂SO₄), and appropriate stirrer speed. Pre-cultures are grown in 25 mL Pyrex Erlenmeyer flasks as described above and incubated for 4 hours post-induction. An appropriate amount of this pre-culture is centrifuged, washed twice with fresh media, and used for inoculation (400 mL initial volume). The fermentations in bioreactor use described fermentation media with 40 g/L glucose and appropriate IPTG and antibiotics. If required, lactic acid (20 mM) is added at 0, 24, and 48 hours.

After the fermentation, the supernatant obtained through 5000 g, 5 min centrifuge in an Optima L-80XP Ultracentrifuge (Beckman-Coulter, Schaumburg, IL) of 2 mL culture is prepared for GC-FID/GC-MS analysis. The supernatant aliquots of 2 mL are transferred to 5 mL glass vials (Fisher Scientific Co., Pittsburgh, PA). Then, organic solvent (typically ethyl acetate) is added at a 1:1 ratio to a fermentation broth sample (e.g. 2 mL for a 2 mL aqueous solution) for extraction. Following an appropriate extraction (vortex samples for 15 seconds, spin on a rotator at 60 rpm for 2 hours, and vortex again for 15 seconds), 1 mL of the organic phase is removed. 50 μL pyridine and 50 uL BSTFA are then added to the 1 mL organic phase for derivatization, with the reaction allowed to proceed at 70° C. for 30 minutes. After cooling to room temperature, this mixture is used for GC analysis.

GC analysis is conducted on an Agilent Intuvo 9000 Series Custom Gas Chromatography system equipped with a 5977B Inert Plus Mass Selective Detector Turbo EI Bundle (for identification) or a Flame Ionization Detector (for quantification) and an Agilent HP-5 capillary column (0.25 mm internal diameter, 0.25 μm film thickness, 30 m length). The following temperature profile is used with helium as the carrier gas at a flowrate of 1.5 mL/min: Initial 50° C. (hold 3 min); ramp at 20° C./min to 270° C. (hold 6 min). The injector and detector temperature are 250° C. and 350° C., respectively. 1 uL of sample is injected with a 20:1 split ratio.

### Example 2

This Example demonstrates the implementation of the carboxylic acid (CA) platform using lactic acid (lactate) as the CA intermediate with C1 elongation pathways. As described above, lactate can be supplied as the carbon source or generated from a carbon source such as glucose and/or other biomass-derived sugars. The activation of lactate and reduction to various compounds such as lactaldehyde, acetone, or propanal proceeds through the reactions and associated enzymes described above.

For C1 elongation, pathways are based on 2-hydroxyacyl-CoA (HACL) enzymes that condense carbonyl-containing compounds, including propanal, lactaldehyde, and acetone, with formyl-CoA (Chou, A., et al. Nat. Chem. Biol. 15:900-906 (2019)). Here these intermediates are selectively generated through the lactate reduction and serve as the substrate for C1 addition, with the resulting 2-hydroxyacyl-CoA converted to various products. Formyl-CoA is generated from formaldehyde through the CoA acylating formaldehyde dehydrogenase from Listeria monocytogenes (Chou, A., et al. Nat. Chem. Biol. 15:900-906 (2019)).

When lactaldehyde is generated from lactoyl-CoA, the HACL from Rhodospirillales bacterium URHD0017 is used to condense lactaldehyde with formyl-CoA forming 2,3-dihydroxybutyryl-CoA. This C4 intermediate can then be converted to various products. 2,3-dihydroxybutyric acid is produced from 2,3-dihydroxybutyryl-CoA through the action of a thioesterase such as TesB from E. coli or Pseudomonas putida (McMahon, M. D. and Prather, K. L. J. Appl. Environ. Microbiol. 80:1042-1050 (2014)). Alternatively, 2,3-dihydroxybutyryl-CoA can be reduced to 2,3-dihydroxybutyraldehyde by an acyl-CoA reductase such as Clostridium beijerinckii ALD (Kim, S., et al. J. Ind.

Microbiol. Biotechnol. 42:465-475 (2015)). 2,3-dihydroxybutyraldehyde can be further reduced to 1,2,3-butanetriol by a suitable alcohol dehydrogenase such as E. coli FucO, AdhP, or YqhD (Kim, S., et al. J. Ind. Microbiol. Biotechnol. 42:465-475 (2015)).

Dehydration of 1,2,3-butanetriol to 3-hydroxybutyraldehyde is catalyzed by the K. oxytoca diol dehydratase (Yamanishi, M., et al. FEBS J. 279: 793-804 (2012)). Further reduction of 3-hydroxybutyraldehyde through the action of a suitable alcohol dehydrogenase such as E. coli FucO, AdhP, or YqhD (Kim, S., et al. J. Ind. Microbiol. Biotechnol. 42:465-475 (2015)) results in the production of 1,3-butanediol.

When acetone is generated from lactoyl-CoA, the HACL from Rhodospirillales bacterium URHD0017 is used to condense acetone with formyl-CoA forming 2-hydroxyisobutyryl-CoA. This C4 intermediate can then be converted to various products, including 2-hydroxyisobutyric acid, 2-hydroxyisobutyraldehyde 2-methyl-1,2-propanediol, isobutyraldehyde, and isobutanol, through the pathways described above using suitable enzymes.

When propanal is generated from lactoyl-CoA, the HACL from Rhodospirillales bacterium URHD0017 is used to condense propanal with formyl-CoA forming 2-hydroxybutyryl-CoA. This C4 intermediate can then be converted to various products, including 2-hydroxybutyric acid, 2-hydroxybutyraldehyde 1,2-butanediol, butyraldehyde, and n-butanol, through the pathways described above using suitable enzymes.

To generate the reducing equivalents and ATP required for reduced product synthesis, methanol is supplied as the external energy source. Methanol oxidation proceeds through the NADH-dependent methanol dehydrogenase 2 from Bacillus methanolicus (Roth, T. B., et al. ACS Syn. Biol. 8:796-806 (2019)) to form formaldehyde and generate a NADH. To enable ATP generation directly from the provided C1, a CoA acylating formaldehyde dehydrogenase, phosphate formyltransferase, and formate kinase are used to convert formaldehyde to formate through formyl-CoA and formyl-phosphate intermediates. Here the CoA acylating formaldehyde dehydrogenase from Listeria monocytogenes (Chou, A., et al. Nat. Chem. Biol. 15:900-906 (2019)) and the phosphate formyltransferase and formate kinase enzymes from Clostridium cylindrosporum (Sly, W. S. and Stadtman, E. R. J. Biol. Chem. 238:2639-2647 (1963)) are used to generate an additional NADH and an ATP during this conversion. Formate oxidation to CO2 through the NADH-dependent formate dehydrogenase from Pseudomonas sp. (strain 101) is used to generate another NADH (Tishkov, V. I., et al. Biochem. Biophys. Res. Commun. 192:976-81 (1993)).

### Example 3: Overview of Enzymes Catalyzing Energy (Redox and ATP) and Formyl-COA Generation

The purpose of this example is to provide exemplary genes, enzymes and pathways involved in the interconversion of one-carbon (C1) molecules to provide energy (in the form of NADH and ATP) and formyl-CoA required for C1 elongation reactions.

While providing preferred embodiments of the invention, these are given as illustration which combined with this disclosure allow a person skilled in the art to make additional modifications. The examples teach how to endow microorganisms with said capabilities, including specific pathway designs, required genes/enzymes to construct said pathways, methods for cloning and transformation, monitoring product formation and using the engineered microorganisms for production. While the examples show modified Escherichia coli strains, these modifications can easily be performed in other microorganisms of the same family Enterobacteriaceae as well as other bacterial species as well as yeast and fungi. Methane is readily available C1 source from natural gas, landfills, and agriculture. Biological oxidation of methane to methanol is catalyzed by methane monooxygenases. Functional expression of soluble methane monooxygenase (sMMO) from Methylococcus capsulatus is demonstrated in E. coli as a host (bioRxiv 2021. 08.05.455234) FIG. 1 and Table 1). Subsequent oxidation of methanol to formaldehyde can be catalyzed by NAD⁺-dependent methanol dehydrogenases (MDH). MDH from Bacillus methanolicus MGA3 (BmMDH), Bacillus stearothermophilus (BsMDH) (Metab. Eng. 39:49-59, 2017) and Cupriavidus necator (Appl. Microbiol. Biotechnol. 100:4969-4983, 2016) are expressed and characterized in E. coli (FIG. 1 and Table 1). Formaldehyde can be directly oxidized to formic acid catalyzed by E. coli formaldehyde detoxification system (frmA). Alternatively, formaldehyde oxidation to formyl-CoA can be catalyzed by various acylating aldehyde dehydrogenase candidates (Nat. Chem. Biol. 15:900-906, 2019) (FIG. 1 and Table 1). Formyl-CoA can either be fed to the C1 elongation platform or further converted to generate energy. Formyl-CoA hydrolysis to formate can be catalyzed by two different families of enzymes. Acyl-CoA transferases (ACT) can catalyze reversible CoA transfer from various CoA donor, such as glycolyl-CoA, acetyl-CoA or succinyl-CoA to formate. Phosphotransacylase-formate kinase (PTA-FOK) pair catalyze reversible phosphorylation of formyl-CoA to formyl-phosphate, followed by dephosphorylation to formate generating 1 ATP. While these two reactions are considered fully reversible, AMP-forming acyl-CoA synthetases are favored toward formate activation to formyl-CoA. Finally, formate can be further oxidized to CO2 to generate NADH, catalyzed by soluble formate dehydrogenases, or the same enzyme can be used to reduce CO2 to formate for formyl-CoA generation (FIG. 1 and Table 1).

TABLE 1
List of enzymes for energy (redox and
ATP) and formyl-CoA generation pathways

| | | | Uniprot |
|---|---|---|---|
| Reaction | Gene | Organism | accession |
| sMMO | mmoXYBZCD | Methylococcus | P18797 |
| | | capsulatus | |
| pMMO | pmoA1A2B1B2 | Methylococcus | G1UBD1 |
| | | capsulatus | Q607G3 |
| MDH | BmMDH2 | Bacillus | I3E2P9 |
| | | methanolicus | |
| | | MGA3 | |
| | CnMDH2 | Cupriavidus | F8GNE5 |
| | | necator | |
| | BsMDH | Bacillus | P42327 |
| | | stearothermophilus | |
| ACR1 | LmACR | Listeria | Q8Y7U1 |
| | | monocytogenes | |
| | StEutE | Salmonella | P41793 |
| | | typhymurium | |
| | CbA1d | Clostridium | Q716S8 |
| | | beijerinckii | |
| | EcMhpF | Escherichia coli | P77580 |
| | PsDmpF | Pseudomonas sp. | Q52060 |
| | | strain CF600 | |
| ALDH | EcFrmA | Escherichia coli | P25437 |
| | PpFdhA | Pseudomonas | P46154 |
| | | putida | |
| PTA-FOK | CcPta-Ack | Clostridium | A0A0J8D6J2 |
| | | cylindrosporum | A0A0J8DB00 |
| | EcPta-Ack | Escherichia coli | P0A9M8 |
| | | | P0A6A3 |
| ACS | EcACS | Escherichia coli | P27550 |
| | StACSstab | Salmonella | Q8ZKF6_PROSS |
| | | typhimurium | |
| | MhACS | Marinithermus | F2NQX2 |
| | | hydrothemalis | |
| | ArACS | Angustibacter sp. | A0A0Q7JEV7 |
| | | Root456 | |
| ACT | CaAbfT | Clostridium | Q9RM86 |
| | | aminobutyricum | |
| | OfFrc | Oxalobacter | O06644 |
| | | formigenes | |
| FDH | PsFdh | Pseudomonas sp. | P33160 |
| | | (strain 101) | |
| | CbFdh | Candida boidinii | O13437 |

### Example 4: Formyl-COA Generation from Formic Acid

The purpose of this example is to demonstrate the aspect of the invention pertaining to generation of formyl-CoA from formate in vivo using both resting cells and growing cultures.

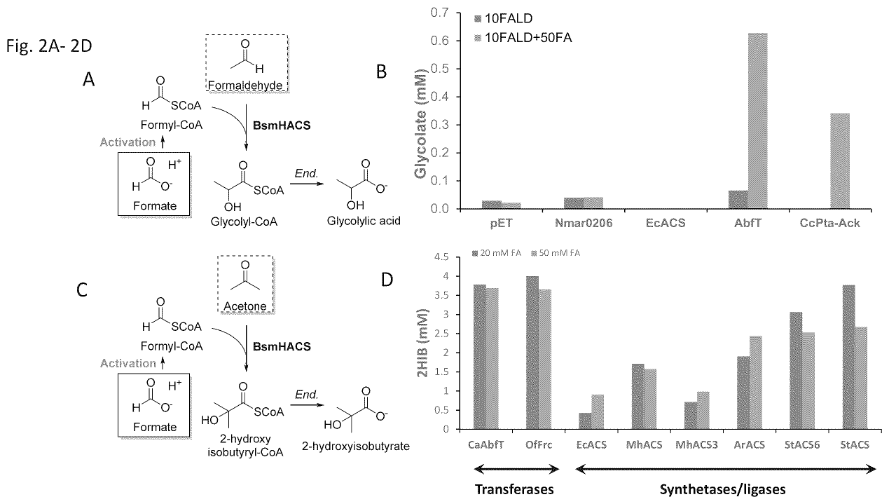

The formyl-CoA generated through formate activation enzymes (FAE) is further condensed with formaldehyde to produce glycolic acid. We engineered vectors to independently control expression of BsmHACS (UniProt accession: A0A3C0TX30), sourced from beach sand metagenome, along with FAEs. BsmHACS is under control of the IPTG-inducible T7 promoter in pCDFduet-1 and FAEs under control of a cumate-inducible T5 promoter in pETDuet-1 (FIG. 2A). FAEs were selected to represent the three major formate activation routes, including acyl-CoA synthase ACS (Nmar0206 from Nitrosopumilus maritimus and EcACS), acyl-CoA transferase ACT (AbfT from Clostridium aminobutyricum) and phosphotransacylase(PTA)-FOK (CcPta+CcAck from Clostridium cylindrosporum). As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD), which we expected could compete or interfere with the analysis of our pathway.

Cells from the above pre-cultures were then centrifuged (5000×g, 22° C.), washed twice with the above minimal media without any carbon source, and resuspended to an optical density ˜10. Five mL of this cell suspension and indicated amounts of carbon source (e.g. formaldehyde) was added to 25 mL Pyrex Erlenmeyer flasks (Corning Inc., Corning, NY) and sealed with foam plugs filling the necks. 10 mM formaldehyde and 50 mM formate were added at 0 hr. Flasks were incubated at 30° C. and 200 rpm in an NBS 124 Benchtop Incubator Shaker (New Brunswick Scientific Co., Inc., Edison, NJ). After incubation at 30° C. for 24 hours, the cells were centrifuged at 20817×g for 15 minutes and the supernatant analyzed by HPLC as described below.

Quantification of product and substrate concentrations (formic acid, formaldehyde and glycolic acid) were determined via HPLC using a Shimadzu Prominence SIL 20 system (Shimadzu Scientific Instruments, Inc., Columbia, MD) equipped with a refractive index detector and an HPX-87H organic acid column (Bio-Rad, Hercules, CA) with operating conditions to optimize peak separation (0.3 ml/min flowrate, 30 mM H₂SO₄ mobile phase, column temperature 42° C.). Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 μm film thickness, 30 m length).

The expected product glycolic acid was detected in the media, indicating the performance of corresponding formate activation enzymes. Of the tested formate CoA transferase CaAbfT has the best performance (FIG. 2B).

Since formate activation is a required step for formate utilization, we further evaluated enzymes catalyzing this reaction. Acyl-CoA synthetase (ACS) enzymes are one of the three explored routes for formate activation, which can directly generate formyl-CoA from formate with the consumption of 2 ATP equivalents (ATP is converted to AMP). ACSs evaluated using resting cells showed relatively poor activity as described above, which may be due to limited availability of ATP in the resting cell experiments. Therefore, ACS variants were further evaluated using the new established growing cell platform with BsmHACS overexpression. In this platform, formyl-CoA generated through formate activation enzymes is further condensed with non-toxic substrate acetone to produce 2-hydroxyisobutyrate (2HIB) (FIG. 2C).

The screening of a larger group of formate activation enzymes in actively growing cells was conducted using the M9-LB medium contains 6.78 g/L Na₂HPO₄, 3 g/L KH₂PO₄, 1 g/L NH₄Cl, 0.5 g/L NaCl, 2 mM MgSO₄, 100 μM CaCl₂), 15 μM thiamine-HCl, 10 g/L tryptone, and 5 g/L yeast extract additionally supplemented with the micronutrient solution of Neidhardt⁶⁸. A single colony of the desired strain was cultivated overnight (14-16 hrs) in LB medium with appropriate antibiotics and used as the inoculum (1%) to 50 mL centrifugation tubes containing 5 mL of M9-LB medium. Antibiotics (100 μg/mL carbenicillin, 100 μg/mL spectinomycin) were included when appropriate. Cultures were then incubated at 30° C. and 250 rpm in a Lab Companion SI-600 rotary shaker (Jeio Tech, Seoul, South Korea) until an OD₅₅₀ of ˜0.4 was reached, at which point appropriate amounts of inducer(s) (isopropyl β-D-1-thiogalactopyranoside and cumate) and substrates (100 mM acetone and 20 mM formate) were added. Tubes were tightened and incubated for a total of 48 hr post-inoculation. The cells were pelleted by centrifugation and the supernatant analyzed by HPLC as described below. The expected product 2-hydroxyisobutyric acid was detected in the media indicating the performance of corresponding formate activation enzymes.

Quantification of product and substrate concentrations (formic acid, acetone and 2HIB) were determined via HPLC using a Shimadzu Prominence SIL 20 system (Shimadzu Scientific Instruments, Inc., Columbia, MD) equipped with a refractive index detector and an HPX-87H organic acid column (Bio-Rad, Hercules, CA) with operating conditions to optimize peak separation (0.3 ml/min flowrate, 30 mM H₂SO₄ mobile phase, column temperature 42° C.). Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 μm film thickness, 30 m length).

To test the ACS activity, the two CoA transferases which have good performance on formate activation were also included to serve as positive controls. By screening a larger group of FAEs in actively growing cells, the CoA transferases (CaAbfT and OfFrc) which have a better performance on formate activation than other tested formate activation enzymes. Of the ACSs tested, StACS has the best performance, its activity being comparable to CoA transferases (FIG. 2D).

### Example 5: Formyl-COA Generation from Formaldehyde

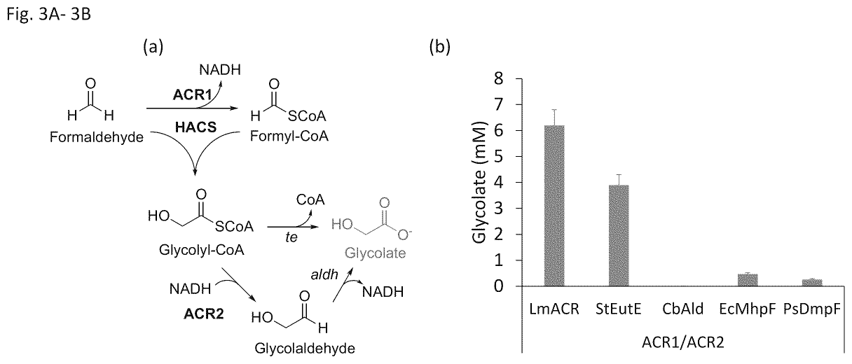

The purpose of this example is to demonstrate the use of various acylating formaldehyde dehydrogenase/acyl-CoA reductase (ACR1) catalyzing interconversion between formaldehyde and formyl-CoA. We used glycolic acid (glycolate) production as proxy, by adding active 2-hydroxyacyl-CoA lyase, HACL from Rhodospirillales bacterium URHD0017 (RuHACL), along with different ACR1 candidates (FIG. 3A).

Cell-free reactions for pathway prototyping contained 50 mM KPi pH 7.4, 4 mM MgCl₂, 0.1 mM TPP, 2.5 mM CoASH, 5 mM NAD⁺, and 50 mM formaldehyde. Individual cell extract loading was around 4.4 g/L protein (1/8 of the reaction volume), and the amount of protein added to each reaction was normalized with BL21(DE3) extract to ˜26 g/L protein (3/4 of the reaction volume). Reactions were incubated at room temperature for one hour unless otherwise specified. 1/4 of the reaction volume of saturated ammonium sulfate solution acidified with 1% sulfuric acid was added to terminate the reactions. Samples were centrifuged at 20817×g for 15 minutes and the supernatant analyzed by HPLC or GC-MS as described below.

Quantification of product and substrate concentrations (formic acid, formaldehyde and glycolic acid) were determined via HPLC using a Shimadzu Prominence SIL 20 system (Shimadzu Scientific Instruments, Inc., Columbia, MD) equipped with a refractive index detector and an HPX-87H organic acid column (Bio-Rad, Hercules, CA) with operating conditions to optimize peak separation (0.3 ml/min flowrate, 30 mM H₂SO₄ mobile phase, column temperature 42° C.). Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 μm film thickness, 30 m length).

We combined extracts of E. coli, prepared as described in the previous example(s), expressing RuHACL and ACR1 variants to rapidly screen for the best performing combinations. Of the ACRs tested, the variant from Listeria monocytogenes (LmACR) was best suited for enabling the pathway. The combination of LmACR and RuHACL enabled the production of 6.2±0.6 mM glycolic acid in 1 hr (FIG. 3B).

### Example 6: Methanol as the Source for Redox (NADH) and Formyl COA Generation

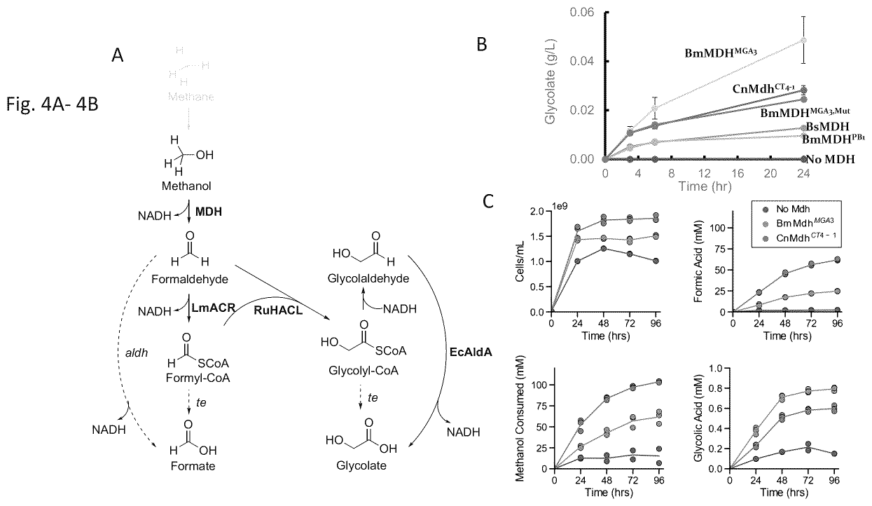

The purpose of this example is to demonstrate the utilization of methanol as source for NADH and formyl-CoA generation demonstrated in vivo with both resting cells and growing cultures. Methanol oxidation to formaldehyde catalyzed by NAD⁺-dependent methanol dehydrogenase generates NADH, which could be used as reducing power for the pathway or energy in the form of ATP when coupled with oxidative phosphorylation. Likewise, formaldehyde oxidation to formyl-CoA also generates one NADH. These two enzymes combined with HACL from Rhodospirillales bacterium URHD0017 (RuHACL) catalyze methanol to glycolyl-CoA, which is readily hydrolyzed to glycolate by endogenously expressed thioesterases (FIG. 4A).

To implement methanol utilization pathway in vivo, we engineered vectors to express RuHACL and the acyl-CoA reductase from Listeria monocytogenes (LmACR) and various methanol dehydrogenase (MDH) candidates. As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD), which we expected could compete or interfere with the analysis of our pathway.

Resting-cell prototyping was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 125 mL baffled flasks (Wheaton, Millville, NJ) containing 25 mL of the above media further supplemented with 20 g/L glycerol, 10 g/L tryptone, and 5 g/L yeast extract. A single colony of the desired strain was cultivated overnight (14-16 hrs) in LB medium with appropriate antibiotics and used as the inoculum (1%). Antibiotics (50 μg/mL carbenicillin, 50 μg/mL spectinomycin) were included when appropriate. Cultures were then incubated at 30° C. and 250 rpm in a Lab Companion SI-600 rotary shaker (Jeio Tech, Seoul, South Korea) until an OD₅₅₀ of ˜0.4 was reached, at which point appropriate amounts of inducer(s) (isopropyl β-D-1-thiogalactopyranoside and cumate) were added. Flasks were incubated for a total of 24 hrs post-inoculation.

Cells from the above pre-cultures were then centrifuged (5000×g, 22° C.), washed twice with the above minimal media without any carbon source, and resuspended to an optical density ˜10.5 mL of this cell suspension and indicated amounts of carbon source (e.g. formaldehyde) was added to 25 mL Pyrex Erlenmeyer flasks (Corning Inc., Corning, NY) and sealed with foam plugs filling the necks. 25 mM acetone and 5 mM formaldehyde were added at 0 hr, with additional 5 mM formaldehyde added at 1, 2, and 3 hrs Flasks were incubated at 30° C. and 200 rpm in an NBS 124 Benchtop Incubator Shaker (New Brunswick Scientific Co., Inc., Edison, NJ). After incubation at 30° C. for 24 hours, the cells were pelleted by centrifugation and the media analyzed. The expected product glycolic acid was detected in the media, indicating production by the engineered organism.

For the growing-cell experiment, the growth media used was M9 (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) additionally supplemented with 500 mM methanol, 10 g/L tryptone, 5 g/L yeast extract and micronutrient solution of Neidhardt et al. An overnight LB culture of each strain was used to inoculate (1%) a 50 mL closed-cap conical tube (Genesee Scientific Co.) containing 5 mL of the above media further supplemented with appropriate antibiotics (50 μg/mL carbenicillin, 50 μg/mL spectinomycin). After approximately 3 hours, gene expression was induced by addition of 0.04 mM isopropyl β-d-1-thiogalactopyranoside (IPTG) and 0.04 mM cumate. Tubes were incubated at 30° C. and 200 rpm in an NBS 124 Benchtop Incubator Shaker (New Brunswick Scientific Co.). Samples (100 μL) were taken every 24, 48, 72 and 96 hours after inoculation for OD₆₀₀ measurement and HPLC analysis as described previously.

The resting-cell experiment shows that the combination of various MDHs and LmACR produces formyl-CoA, as indicated by glycolate production as proxy. Of variants tested, BmMDH^{MGA3} gives the best glycolate production of up to 50 mg/L, followed by CnMDH^{CT4-1} of 30 mg/L under the resting-cell format (FIG. 4B).

The best two enzyme candidates were tested under the growing-cell experiment. The correlation between methanol consumption and cell growth indicates the utilization of methanol as source of energy (ATP) via respiration of excess NADH generation. The result with glycolate production aligns with the result from resting-cell experiments but CnMDH^{CT4-1} shows faster consumption of methanol and accumulation of formate (FIG. 4C). Therefore, BmMDH^{MGA3} is a better choice for the formyl-CoA generation whereas CnMDH^{CT4-1} is a better enzyme for energy generation.

### Example 7: Carboxylic Acid Platform Using Formic Acid

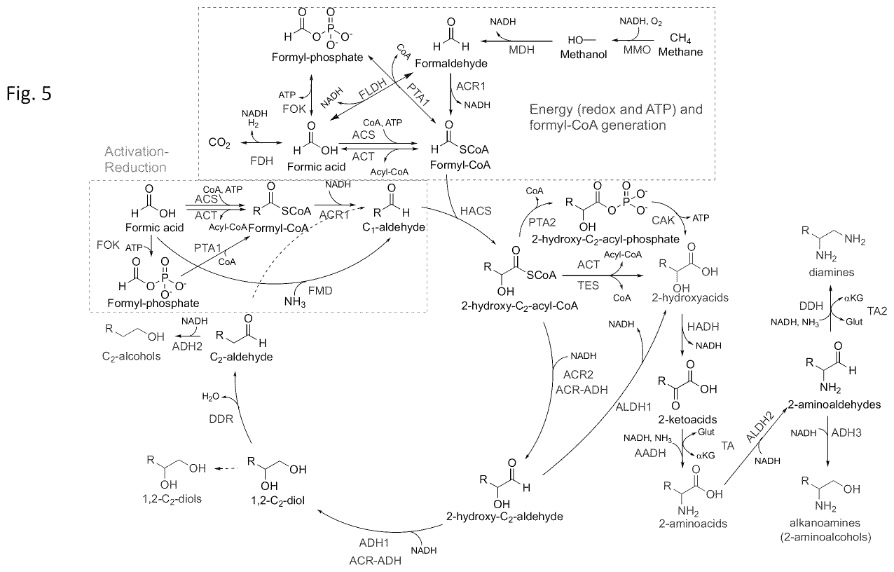

This example demonstrates the implementation of the carboxylic acid (CA) platform using formic acid (formate) as the CA with C1 elongation pathways. Formate can be supplied as the carbon source, internally generated by the cells through the enzyme pyruvate formate lyase (e.g. PFL1 from Chlamydomonas reinhardtii, pf1 from Clostridium pasteurianum, pf1B and tdcE from Escherichia coli, pf1 from Streptococcus mutans: Nucleic Acids Research 42(1): D459-D471, 2014), generated from electrochemical or enzymatic reduction of CO2. Activation and reduction or amination of formate gives (substituted) C1 aldehydes which serve as substrates for the condensation reaction with formyl-CoA. The resulting 2-hydroxyacyl-CoAs can be hydrolyzed to the corresponding acids, which can then be aminated to form amino acids. Reduction of amino acids to aldehydes gives 2-aminoaldehydes, which could subsequently be aminated to form diamines (catalyzed by diamine dehydrogenase or transaminase) or further reduced to form alkanoamines (2-aminoalcohols) (catalyzed by alcohol dehydrogenase). Alternatively, they can go through α-reduction cycle to generate 1,2-diols and alcohols (FIG. 5).

The activation of formate to formyl-CoA is demonstrated using various native and engineered acyl-CoA synthetases (Synth. Biol. 6:1-14, 2021), CoA transferases (J. Biol. Chem. 283: 6519-6529, 2008; ACS Catal. 11:5396-5404, 2021; Nat. Metab. 3:1385-1399, 2021), and carboxylic acid kinase-phosphotransacylase (J. Biol. Chem. 238:2639-2647, 1963). Further reduction of formyl-CoA to formaldehyde is catalyzed by CoA acylating formaldehyde dehydrogenases (e.g. from Listeria monocytogenes: Synth. Biol. 6:1-14, 2021; Nat. Metab. 3:1385-1399, 2021).

When formaldehyde is generated from formyl-CoA, the HACL from Rhodospirillales bacterium URHD0017 can be used to condense formaldehyde with formyl-CoA forming glycolyl-CoA. Glycolyl-CoA can be utilized as CoA donor for activation of formate as demonstrated using AbfT from Clostridium aminobutyricum, or hydrolyzed through the action of a thioesterase such as YciA from E. coli (ACS Catal. 11:5396-5404, 2021) to yield glycolic acid (glycolate). In addition, it can undergo glycolyl-phosphate intermediate to generate glycolate and ATP similar to formic acid activation via phosphate intermediate catalyzed by E. coli Pta and Ack or equivalent enzymes. Glycolate can further be converted to glyoxylate via GlcD from E. coli, followed by alanine dehydrogenase reaction to generate glycine, one of the essential amino acids for living organisms. Multiple alanine dehydrogenases have activity with glyoxylate to yield glycine (J. Bacteriol. 194:1045-1054, 2012; Biochemistry 20:5650-5655, 1981). Glycine can be reduced to aminoacetaldehyde catalyzed by various aldehyde dehydrogenases/oxidases. The resulting aminacetaldehyde can be further aminated via diamine dehydrogenase or transaminase activity to form ethylene diamines or reduced to form ethanolamine.

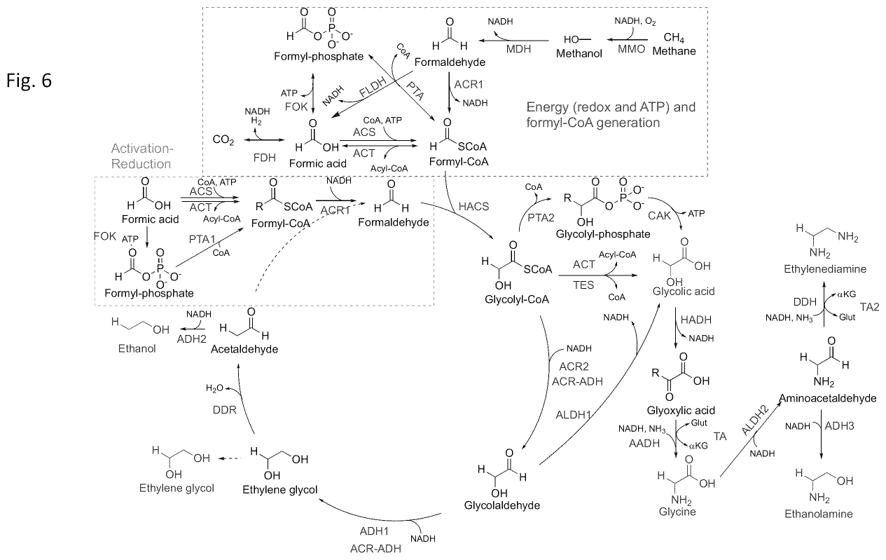

Glycolyl-CoA can be further reduced to glycolaldehyde via acyl-CoA reductase. In this case the same enzyme (LmACR) used for reduction of formyl-CoA to formaldehyde can catalyze reduction of glycolyl-CoA to glycolaldehyde (Nat. Metab. 3:1385-1399 (2021)). Glycolaldehyde reduction to ethylene glycol is catalyzed by E. coli fucO. Dehydration of ethylene glycol gives acetaldehyde, catalyzed by PddABC from Klebsiella oxytoca. The resulting acetaldehyde can be fed to the subsequent iteration of C1 elongation or reduced to ethanol catalyzed by E. coli adhE (FIG. 6 and Table 2).

TABLE 2
List of enzymes responsible for carboxylic
acid platform using formaldehyde as the intermediate
aldehyde for the C1 elongation.

| | | | Uniprot |
|---|---|---|---|
| Reaction | Gene | Organism | accession |
| PTA-FOK | CcPta-Ack | Clostridium | A0A0J8D6J2 |
| | | cylindrosporum | A0A0J8DB00 |
| | EcPta-Ack | Escherichia coli | P0A9M8 |
| | | | P0A6A3 |
| ACS | EcACS | Escherichia coli | P27550 |
| | StACSstab | Salmonella | Q8ZKF6_PROSS |
| | | typhimurium | |
| | MhACS | Marinithermus | F2NQX2 |
| | | hydrothemalis | |
| | ArACS | Angustibacter sp. | A0A0Q7JEV7 |
| | | Root456 | |
| ACT | CaAbfT | Clostridium | Q9RM86 |
| | | aminobutyricum | |
| | OfFrc | Oxalobacter | O06644 |
| | | formigenes | |
| ACR1 | LmACR | Listeria | Q8Y7U1 |
| | | monocytogenes | |
| HACS | RuHACL | Rhodospirillales | A0A1H8YFL8 |
| | | bacterium | |
| | | URHD0017 | |
| | BsmHACL | Beach Sand | A0A3C0TX30 |
| | | Metgenome | |
| | AcHACL | Actinomycetospora | UPI001EAE83D2 |
| | | chiangmaiensis | |
| | | DSM 45062 | |
| | MeOXC4 | Methylorubrum | C5AX46 |
| | | extorquens AM1 | |
| ACR2 | LmACR | Listeria | Q8Y7U1 |
| | | monocytogenes | |
| | StPduP | Salmonella | Q9XDN1 |
| | | typhimurium | |
| ALD | EcAldA | Escherichia coli | P25553 |
| ADH1 | EcfucO | Escherichia coli | P0A9S1 |
| DDR | KoPddABC | Klebsiella oxytoca | Q59470 |
| | | | Q59471 |
| | | | Q59472 |
| ADH2 | EcAdhE | Escherichia coli | P0A9Q7 |
| PTA2-CAK | CcPta-Ack | Clostridium | A0A0J8D6J2 |
| | | cylindrosporum | A0A0J8DB00 |
| | EcPta-Ack | Escherichia coli | P0A9M8 |
| | | | P0A6A3 |
| TES | EcYciA | Escherichia coli | P0A8Z0 |
| HADH | EcGlcD | Escherichia coli | P0AEP9 |
| AADH | MtAld | Mycobacterium | P9WQB1 |
| | | tuberculosis | |
| | BsAld | Bacillus subtilis | Q08352 |
| TA1 | ALAT1 | Homo Sapiens | P24298 |
| ALDH2 | aldH1 | Aquifex aeolicus | O66573 |
| | dhaS | Anoxybacillus | B7GJB2 |
| | | flavithermus | |
| TA2 | EcSerC | Escherichia coli | P23721 |
| | GOT1 | Sus scrofa | P00503 |
| ADH3 | EcYdfG | Escherichia coli | P39831 |

### Example 8: Conversion of Formate to Ethylene Glycol

This Example demonstrates the implementation of the carboxylic acid (CA) platform using formic acid (formate) as the CA intermediate with C1 elongation pathways. The activation of formate to formyl-CoA is demonstrated using various native and engineered acyl-CoA synthetases (Synth. Biol. 6:1-14, 2021) and CoA transferases (J. Biol. Chem. 283: 6519-6529, 2008; ACS Catal. 11:5396-5404, 2021; Nat. Metab. 3:1385-1399, 2021). Further reduction of formyl-CoA to formaldehyde is catalyzed by CoA acylating formaldehyde dehydrogenases (e.g., from Listeria monocytogenes: Synth. Biol. 6:1-14, 2021; Nat. Metab. 3:1385-1399, 2021).

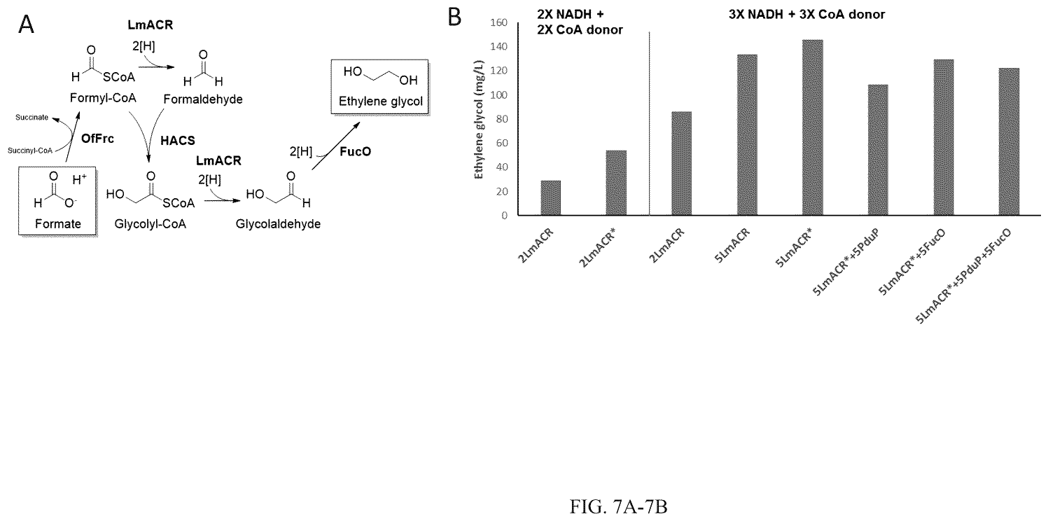

When formaldehyde is generated from formyl-CoA, the HACL from Rhodospirillales bacterium URHD0017 (RuHACL) or beach sand metagenome BsmHACS (UniProt accession: A0A3C0TX30) is used to condense formaldehyde with formyl-CoA forming glycolyl-CoA. Glycolyl-CoA can be further reduced to glycolaldehyde via acyl-CoA reductase. In this case the same enzyme (LmACR) used for reduction of formyl-CoA to formaldehyde can catalyze reduction of glycolyl-CoA to glycolaldehyde (Nat. Metab. 3:1385-1399, 2021). Glycolaldehyde reduction to ethylene glycol is catalyzed by E. coli fucO (FIG. 7A).

Expression of selected enzyme variants was achieved using plasmid-based gene expression by cloning the desired gene(s) into pETDuet-1 or pCDFDuet-1 (Novagen, Darmstadt, Germany) digested with appropriate restriction enzymes and by utilizing In-Fusion cloning technology (Clontech Laboratories, Inc., Mountain View, CA). Linear DNA fragments for insertion were created via PCR of the open reading frame of interest (for genes native to E. coli) or by gene synthesis of the codon optimized gene. Genes were synthesized by GeneArt (Life Technologies, Carlsbad, CA). Resulting In-Fusion reaction products were used to transform E. coli Stellar cells (Clontech Laboratories, Inc., Mountain View, CA), and clones identified by PCR screening were further confirmed by DNA sequencing.

Overnight cultures of the expression strains were grown in LB, which was used to inoculate 25 mL TB medium in a 250 mL baffled flask at 1%. The culture was grown at 30° C. and 250 rpm in an orbital shaker until OD550 reached 0.4-0.6, at which point expression was induced with 0.1 mM IPTG. 24 hours post inoculation, cells were harvested by centrifugation. The cell pellets were washed once with cold 9 g/L NaCl solution and stored at −80° C. until needed. Antibiotics were included where appropriate at the following concentrations: ampicillin (100 μg/mL), carbenicillin (50 μg/mL), and spectinomycin (50 μg/mL).

For protein purification, E. coli cell pellets expressing the desired his-tagged enzymes were prepared as described above. The frozen cell pellets were resuspended in cold lysis buffer (50 mM NaPi pH 7.4, 300 mM NaCl, 10 mM imidazole, 0.1% Triton-X 100) to an approximate OD550 of 40, to which 1 mg/mL of lysozyme and 250 U of Benzonase nuclease was added. The mixture was further treated by sonication on ice using a Branson Sonifier 250 (5 minutes with a 25% duty cycle and output control set at 3), and centrifuged at 7500×g for 15 minutes at 4° C. The supernatant was applied to a chromatography column containing 1 mL TALON metal affinity resin (Clontech Laboratories, Inc., Mountain View, CA), which had been pre-equilibrated with the lysis buffer. The column was then washed first with 10 mL of the lysis buffer and then twice with 20 mL of wash buffer (50 mM NaPi pH 7.4, 300 mM NaCl, 20 mM imidazole). The his-tagged protein of interest was eluted with 1-2 applications of 4 mL elution buffer (50 mM NaPi pH 7.4, 300 mM NaCl, 250 mM imidazole). The eluate was collected and applied to a 10,000 MWCO Amicon ultrafiltration centrifugal device (Millipore, Billerica, MA), and the concentrate (˜100 μL) was washed twice with 4 mL of 50 mM KPi pH 7.4 for desalting. Protein concentrations were estimated by the Bradford method. Purified protein was saved in 20 μL aliquots at −80° C. until needed. SDS-PAGE was performed using NuPAGE 12% Bis-Tris Protein Gels with SDS running buffer and stained with SimplyBlue SafeStain according to manufacturer protocols (ThermoFisher Scientific, Waltham, MA).

In vitro purified enzyme reactions for products from formate as a sole carbon source was comprised of 200 mM KPi pH 8.0, 5 mM MgCl₂, 0.2 mM TPP, 6 mM NADH, 2 mM succinyl-CoA, 2 μM BsmHACL or RuHACL^{G390N}, 2 M OfFrc, 4 μM LmACR, 2 μM FucO and 50 mM sodium formate. Reactions were incubated at 30° C. for 24 hours unless otherwise specified. 1/20 of the reaction volume of 10 M NaOH solution was added to terminate the reactions. After 30 min hydrolysis, 1/20 of the reaction volume of 10 N H₂SO₄ was added to neutralize the pH. Samples were centrifuged at 20817×g for 15 minutes and the supernatant analyzed by HPLC or GC-MS as described below.

Quantification of product and substrate concentrations (formic acid, formaldehyde, glycolic acid and ethylene glycol) was performed via HPLC using a Shimadzu Prominence SIL 20 system (Shimadzu Scientific Instruments, Inc., Columbia, MD) equipped with a refractive index detector and an HPX-87H organic acid column (Bio-Rad, Hercules, CA) with operating conditions to optimize peak separation (0.3 ml/min flowrate, 30 mM H₂SO₄ mobile phase, column temperature) 42° C. Compound identification and analysis was performed by GC-MS using an Agilent 7890B Series Custom Gas Chromatography system equipped with a 5977B Inert Plus Mass Selective Detector (for identification) and an Agilent HP-INNOWax Columns (0.25 mm internal diameter, 0.25 μm film thickness, 30 m length).

By using the best identified ACR enzyme LmACR*, ethylene glycol titer was improved 85% compared to the wide type LmACR. By adding more LmACR*, providing more reducing equivalent NADH and more CoA donor, the ethylene glycol titer was further increased to 145.8 mg/L (FIG. 7). The in vitro experiments demonstrate that LmACR and NADH/reducing equivalents have a significant impact on formate conversion to ethylene glycol (FIG. 7B).

### Example 9: Formaldehyde-Formyl-COA Condensation In Vivo

The purpose of this example is to demonstrate the aspect of the invention pertaining to C1-C1 (formaldehyde-formyl-CoA) condensation for product synthesis in vivo.

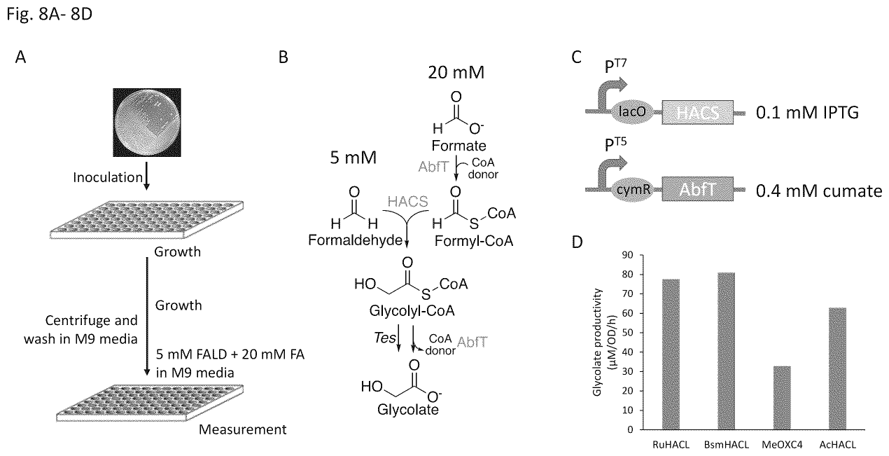

To implement the C1 elongation pathway for glycolate production in vivo, we engineered vectors to independently control expression of various HACS candidates and the acyl-CoA transferase from Clostridium aminobutyricum (CaAbfT), with HACS under control of the IPTG-inducible T7 promoter in pETDuet-1 and CaAbfT under control of a cumate-inducible T5 promoter in pCDFDuet-1 (FIG. 8C). As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD), which we expected could compete or interfere with the analysis of our pathway (FIG. 8B).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.2 mL of the above media further supplemented with 20 g/L glycerol, 10 g/L tryptone, and 5 g/L yeast extract. A single colony of the desired strain was cultivated overnight (14-16 hrs) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hrs post-inoculation.

Cells from the above pre-cultures were then centrifuged (4000 rpm, 22° C.), washed with the above minimal media without any carbon source, and resuspended with 1 mL of above minimal media containing indicated amounts of carbon source. 5 mM formaldehyde and 20 mM formate were added at 0 hr and were incubated at 30° C. and 1000 rpm in Digital Microplate Shaker (Fisher Scientific). After incubation at 30° C. for 3 hours, the cells were pelleted by centrifugation and the media analyzed (FIG. 8A).

The result indicates that RuHACL and BsmHACL are the two best candidates under the given experimental conditions reaching up to 80 μM/h per OD600.

### Example 10: Cell-Free Pathway Prototyping Using Formaldehyde as Starting Substrate

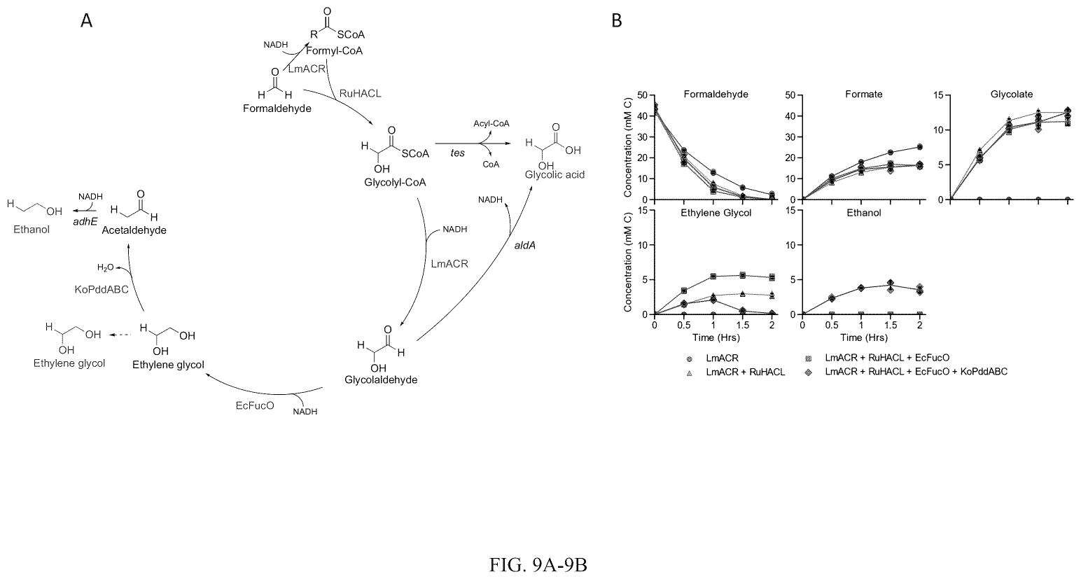

The purpose of this example is to demonstrate the cell-free method for prototyping C1 elongation pathway for formaldehyde as a starting substrate, which could be generated through activation and reduction of carboxylic acid, formate. We prototyped the use of enzymes identified in the previous example as part of a synthetic pathway for the conversion of C1 substrates to multi-carbon products using formaldehyde as the sole carbon source (FIG. 9A). The most immediate two-carbon ligation product of the pathway is glycolic acid, which can be readily hydrolyzed from glycolyl-CoA by endogenously expressed thioesterases. Thus, the synthesis of glycolic acid from formaldehyde using HACL only requires the additional generation of formyl-CoA, which can be accomplished by the use of an acyl-CoA reductase (ACR).

Cell-free reactions for pathway prototyping contained 50 mM KPi pH 7.4, 4 mM MgCl₂, 0.1 mM TPP, 2.5 mM CoASH, 5 mM NAD⁺, and 50 mM formaldehyde. For time course experiments, 0.1 mM coenzyme B12 was added. Individual cell extract loading was around 4.4 g/L protein (1/8 of the reaction volume), and the amount of protein added to each reaction was normalized with BL21(DE3) extract to ˜26 g/L protein (3/4 of the reaction volume). Reactions were incubated at room temperature for one hour unless otherwise specified. 1/4 of the reaction volume of saturated ammonium sulfate solution acidified with 1% sulfuric acid was added to terminate the reactions. Samples were centrifuged at 20817×g for 15 minutes and the supernatant analyzed by HPLC or GC-MS as described previously.

To demonstrate the utility of the HACL-catalyzed elongation reaction for generating varied chemical functionalities from the resulting 2-hydroxyacyl-CoA product, we included enzymes to extend the LmACR+RuHACL pathway (FIG. 9B). An acyl-CoA reductase is needed to reduce glycolyl-CoA to glycolaldehyde, and upon screening the same set of ACRs for activity on glycolaldehyde, we found that LmACR was also able to act upon glycolaldehyde. To minimize the complexity of the engineered system, we used LmACR in a bifunctional role, catalyzing both the oxidation of formaldehyde to formyl-CoA and the reduction of glycolyl-CoA to glycolaldehyde. As shown in FIG. 9B, LmACR alone resulted in only the conversion of formaldehyde to formate. With the inclusion of RuHACL, glycolate was observed. Glycolaldehyde, however, was not significantly detected as a product, probably due to the presence of endogenous oxidoreductases in the cell extract system, which catalyzed the oxidation of glycolaldehyde to glycolic acid or, to a lesser extent, reduction to ethylene glycol.

The synthesis of the next reduction product, ethylene glycol, was significantly increased by the addition of a cell extract of E. coli overexpressing E. coli FucO, a 1,2-diol oxidoreductase (a 2-fold increase, from 1.37±0.1 mM to 2.73±0.03 mM) (FIG. 9B). Ethylene glycol can be further dehydrated to acetaldehyde by a diol dehydratase. Upon addition of E. coli cell extract expressing diol dehydratase (DDR) from Klebsiella oxytoca, ethanol was detected (1.90±0.03 mM at one hour: FIG. 9B), a product of the reduction of acetaldehyde by endogenous aldehyde reductases, along with a corresponding decrease in ethylene glycol. Synthesis of these varied products (i.e. glycolate, ethylene glycol, ethanol) illustrates the use of the 2-hydroxyacyl-CoA node to readily generate products at varying levels of reduction, chain lengths, and functionalities.

### Example 11: Production of Glycine from Glycolate in Glycine Auxotroph Strain

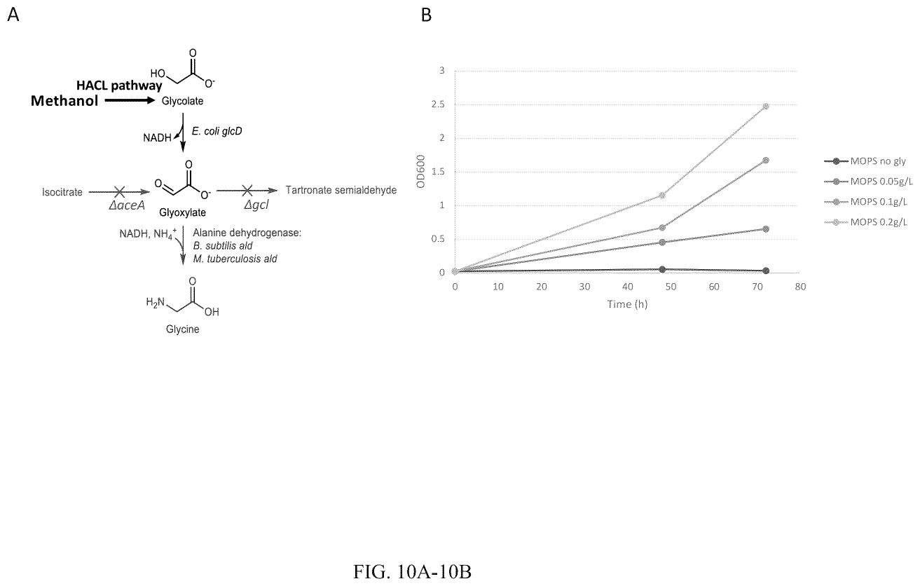

This Example illustrates the alternative route of 2-hydroxyacids to amino acid production when formate is used as the starting carboxylic acid. The demonstration of glycolate conversion to glycine was done through growth-coupled selection of two candidates for alanine dehydrogenases catalyzing glyoxylate reduction to glycine (FIG. 10A). As a host for the selection platform, we engineered a glycine auxotroph strain of E. coli based on MG1655(DE3) with knockouts for glycine production and utilization (ΔaceA Δkbl ΔltaE ΔglyA), which forced the strain to grow only in with the glycine supplementation (FIG. 10A).

For gene deletions, CRISPR is used based on the method developed in Appl. Environ. Microbiol. 81:2506-2514, 2015). First, the host strain is transformed with plasmid pCas, the vector for expression of Cas9 and A-red recombinase. The resulting strain is grown under 30° C. with L-arabinose for induction of A-red recombinase expression, and when OD reaches ˜0.6, competent cells are prepared and transformed with pTargetF (AddGene 62226) expressing sgRNA and N20 spacer targeting the locus and template of insertion of target gene. The template is the deleted gene with ˜500 bp sequences homologous with upstream and downstream of the insertion locus, constructed through overlap PCR with usage of Phusion polymerase or synthesized by GenScript (Piscataway, NJ). The way to switch N20 spacer of pTargetF plasmid is inverse PCR with the modified N20 sequence hanging at the 5′ end of primers with usage of Phusion polymerase and followed by self-ligation with usage of T4 DNA ligase and T4 polynucleotide kinase (New England Biolabs, Ipswich, MA, USA). Transformants that grow under 30° C. on solid media (LB+Agar) supplemented with spectinomycin and kanamycin (or other suitable antibiotic) are isolated and screened for the chromosomal gene insert by PCR. The sequence of the gene insert, which is amplified from genomic DNA through PCR using Phusion polymerase, is further confirmed by DNA sequencing. The pTargetF can then be cured through IPTG induction, and pCas can be cured through growth under higher temperature like 37-42° C.

The resulting glycine auxotroph strain was transformed with a vector constitutively expressing alanine dehydrogenase from Mycobacterium tuberculosis (MtAld) or Bacillus subtilis (BsAld). Out of the two candidates, a strain harboring BsAld started to grow with glycolate instead of glycine supplementation (FIG. 10B) indicating glycolate is successfully being converted to glycine via native glcD and heterologously expressed BsAld genes.

### Example 12: Formamide as Substrate for Condensation with Formyl-COA

This Example demonstrates the implementation of the carboxylic acid (CA) platform using formic acid (formate) as the CA intermediate with C1 elongation pathways. Amination of formate catalyzed by formamidase gives formamide which can also be used as substituted C1 aldehyde substrate for the 2-hydroxyacyl-CoA synthase (HACS) reaction. Functional expression and characterization of formamidases from Methylophilus methylotrophus are demonstrated in literature (Eur. J. Biochem. 240:314-322 (1996)).

The subsequent C1 elongation of formamide catalyzed by HACS forms 2-aminolactoyl-CoA. 2-aminolactoyl-CoA can be utilized as CoA donor for activation of formate using CoA transferases such as AbfT from Clostridium aminobutyricum, or hydrolyzed through the action of a thioesterase such as YciA from E. coli to yield 2-hydroxyglycine. In addition, it can undergo 2-aminolactoyl-phosphate intermediate to generate 2-hydroxyglycine and ATP similar to formic acid activation via phosphate intermediate catalyzed by E. coli Pta and Ack or equivalent enzymes. 2-hydroxyglycine can further be converted to oxamic acid via GlcD from E. coli or similar enzyme, followed by amino acid dehydrogenase reaction to generate diaminoacetic acid. Diaminoacetic acid can be reduced to diaminoethanal catalyzed by various aldehyde dehydrogenases/oxidases. The resulting diaminoethanal can be further aminated via diamine dehydrogenase or transaminase activity to form 1,1,2-ethanetriamine or reduced to form 2,2-diaminoethanol.

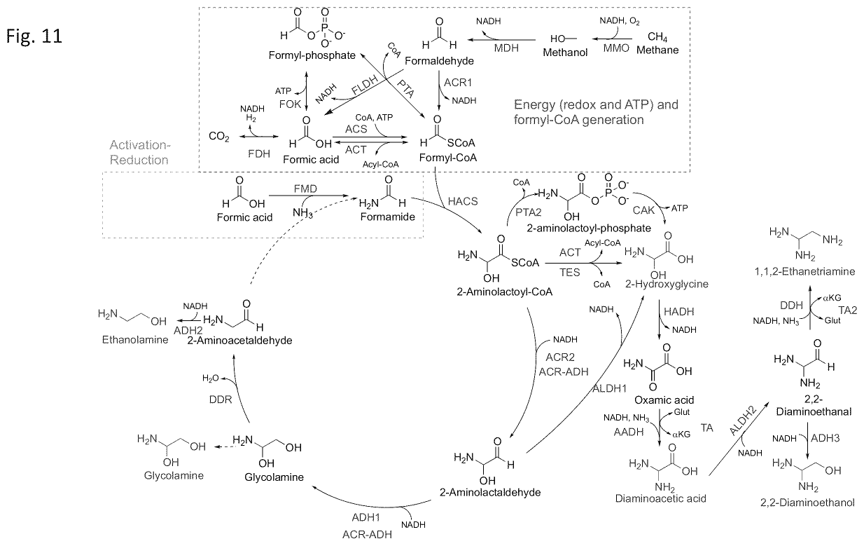

2-aminolactoyl-CoA can be further reduced to 2-aminolactaldehyde via acyl-CoA reductase activity. 2-aminolactaldehyde reduction to glycolamine is catalyzed by E. coli fucO or similar enzymes. Dehydration of glycolamine gives 2-aminoacetaldehyde, catalyzed by diol dehydratases. The resulting 2-aminoacetaldehyde can be fed to the subsequent iteration of C1 elongation or reduced to ethanolamine catalyzed by alcohol dehydrogenases (FIG. 11 and Table 3).

TABLE 3
List of enzymes responsible for carboxylic acid platform using
formamide as the intermediate aldehyde for the C1 elongation.

| | | | Uniprot |
|---|---|---|---|
| Reaction | Gene | Organism | accession |
| FMD | MmFmdA | Methylophilus | Q50228 |
| | | methylotrophus | |
| | | (Bacterium W3A1) | |
| HACS | RuHACL | Rhodospirillales | A0A1H8YFL8 |
| | | bacterium | |
| | | URHD0017 | |
| | BsmHACL | Beach Sand | A0A3C0TX30 |
| | | Metgenome | |
| | AcHACL | Actinomycetospora | UPI001EAE83D2 |
| | | chiangmaiensis | |
| | | DSM 45062 | |
| | MeOXC4 | Methylorubrum | C5AX46 |
| | | extorquens AM1 | |
| ACR2 | LmACR | Listeria | Q8Y7U1 |
| | | monocytogenes | |
| ALD | EcAldA | Escherichia coli | P25553 |
| ADH1 | EcfucO | Escherichia coli | P0A9S1 |
| DDR | KoPddABC | Klebsiella oxytoca | Q59470 |
| | | | Q59471 |
| | | | Q59472 |
| ADH2 | EcAdhE | Escherichia coli | P0A9Q7 |
| PTA2-CAK | CcPta-Ack | Clostridium | A0A0J8D6J2 |
| | | cylindrosporum | A0A0J8DB00 |
| HADH | EcPta-Ack | Escherichia coli | P0A9M8 |
| | | | P0A6A3 |
| TES | EcYciA | Escherichia coli | P0A8Z0 |
| HADH | EcGlcD | Escherichia coli | P0AEP9 |
| AADH | MtAld | Mycobacterium | P9WQB1 |
| | | tuberculosis | |
| | BsAld | Bacillus subtilis | Q08352 |
| TA1 | ALAT1 | Homo Sapiens | P24298 |
| ALDH2 | aldH1 | Aquifex aeolicus | O66573 |
| | dhaS | Anoxybacillus | B7GJB2 |
| | | flavithermus | |
| TA2 | EcSerC | Escherichia coli | P23721 |
| | GOT1 | Sus scrofa | P00503 |
| ADH3 | EcYdfG | Escherichia coli | P39831 |
| | EcGldA | Escherichia coli | P0A9S5 |

### Example 13: Construction of Formamidase Expression Vector

This Example demonstrates the design and construction of a vector used for expression of Methylophilus methylotrophus formamidase (FmdA).

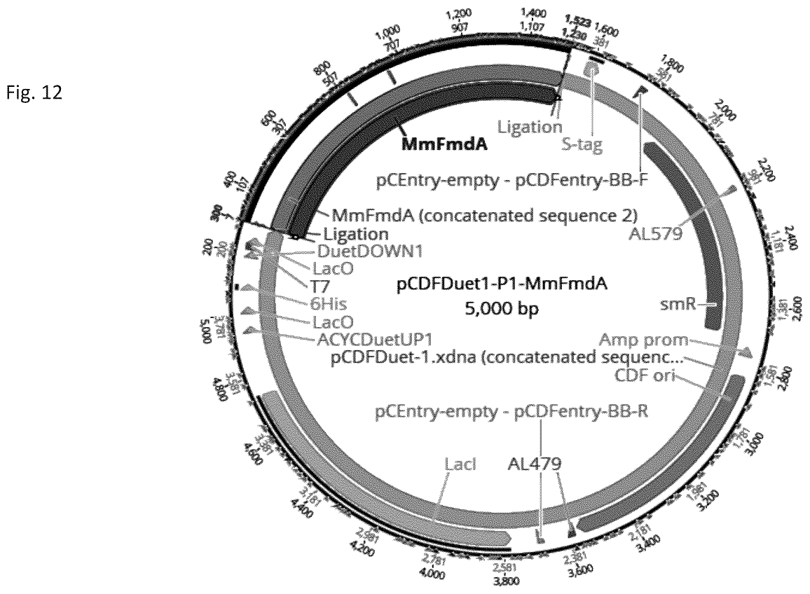

FmdA gene is codon-optimized and synthesized by Twist Biosciences. Then, the gene is amplified through PCR using appropriate primers to append homology on each end for recombination into the vector backbone with e.g., Phusion polymerase (Thermo Scientific, Waltham, MA) to serve as the gene insert. Plasmids are linearized by the appropriate restriction enzymes (New England Biolabs, Ipswich, MA, USA) and recombined with the gene inserts using the In-Fusion HD Eco-Dry Cloning system. The mixture is subsequently transformed into Stellar competent cells. Transformants that grow on solid media (LB+Agar) supplemented with the appropriate antibiotic are isolated and screened for the gene insert by PCR. Plasmids from verified transformants are isolated and the sequence of the gene insert is further confirmed by DNA sequencing. The sequence confirmed plasmids are then introduced to host strain through electroporation (FIG. 12).

### Example 14: Carboxylic Acid Platform Using C₂+ Carboxylic Acids as the CA Intermediate with C1 Elongation Pathways

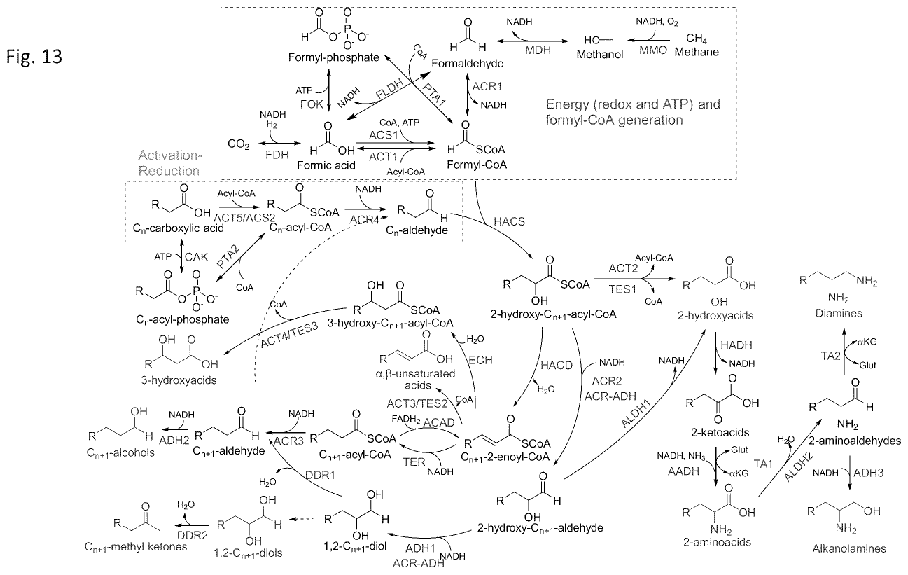

This Example demonstrates the implementation of the carboxylic acid (CA) platform using C₂+ carboxylic acids as the CA intermediate with C1 elongation pathways. C₂+ carboxylic acids can be supplied as the carbon source. Activation and reduction C₂+ carboxylic acids give C₂+ aldehydes which serve as substrate for the condensation reaction with formyl-CoA. The resulting 2-hydroxyacyl-CoAs can be hydrolyzed to the corresponding 2-hydroxyacids, which can then be aminated to form 2-amino acids, alkanolamines and diamines. Alternatively, 2-hydroxyacids can go through α-reduction cycle to generate 1,2-diols, methyl ketones, alcohols, 3-hydroxyacids and α,β-unsaturated acids (FIG. 13).

TABLE 4
List of enzymes for energy (redox and
ATP) and formyl-CoA generation pathways

| | | | Uniprot |
|---|---|---|---|
| Reaction | Gene | Organism | accession |
| MMO | mmoB | Methylococcus | P18797 |
| | | capsulatus | |
| MDH | mdh2 | Bacillus | I3E2P9 |
| | | methanolicus | |
| | mdh2 | Cupriavidus | F8GNE5 |
| | | necator | |
| | adh | Bacillus | P42327 |
| | | stearothermophilus | |
| FLDH | frmA | Escherichia coli | P25437 |
| ACR1 | LmACR | Listeria | Q8Y7U1 |
| | | monocytogenes | |
| | mhpF | Escherichia coli | P77580 |
| | dmpF | Pseudomonas sp. | Q52060 |
| | | CF600 | |
| | eutE | Salmonella | P41793 |
| | | enterica | |
| PTA-FOK | CcPta- | Clostridium | A0A0J8D6J2 |
| | AcK | cylindrosporum | A0A0J8DB00 |
| | EcPta- | Escherichia coli | P0A9M8 |
| | AcK | | P0A6A3 |
| ACS1 | EcACS | Escherichia coli | P27550 |
| | StACSstab | Salmonella | Q8ZKF6 |
| | | typhimurium | |
| | | (strain LT2) | |
| | MhACS | Marinithermus | F2NQX2 |
| | | hydrothermalis | |
| | ArACS | Angustibacter sp. | A0A0Q7JEV7 |
| | | Root456 | |
| ACT1 | abfT | Clostridium | Q9RM86 |
| | | aminobutyricum | |
| | frc | Oxalobacter | O06644 |
| | | formigenes | |
| FDH | fdh | Pseudomonas sp. | P33160 |
| | | (strain 101) | |

TABLE 5
List of enzymes for action-reduction and C1 elongation pathways

| | | | Uniprot |
|---|---|---|---|
| Reaction | Gene | Organism | accession |
| CAK | ackA | Escherichia coli | P0A6A3 |
| | tdcD | Escherichia coli | P11868 |
| | buk1 | Clostridium | Q45829 |
| | | acetobutylicum | |
| PTA2 | pta | Escherichia coli | P0A9M8 |
| | ptb | Clostridium | P58255 |
| | | acetobutylicum | |
| ACS2 | yfaC | Escherichia coli | P27550 |
| | prpE | Escherichia coli | P77495 |
| | lvaE | Pseudomonas | Q88J54 |
| | | putida KT2440 | |
| | AAE3 | Arabidopsis | Q9SMT7 |
| | | thaliana | |
| | sucC | Escherichia coli | P0A836 |
| | sucD | | P0AGE9 |
| ACT5 | cat1 | Clostridium kluyveri | P38946 |
| | scpC | Escherichia coli | P52043 |
| | cat3 | Clostridium kluyveri | A5N390 |
| | yfdE | Escherichia coli | P76518 |
| | pct | Megasphaera | G0VND6 |
| | | elsdenii | |
| ACR4 | eutE | Escherichia coli | P77445 |
| | pduP | Salmonella | Q9XDN1 |
| | | typhimurium (strain | |
| | | LT2) | |
| | adhE2 | Clostridium | Q9ANR5 |
| | | acetobutylicum | |
| | sucD | Clostridium kluyveri | P38947 |
| HACS | RuHACL | Rhodospirillales | A0A1H8YFL8 |
| | | bacterium | |
| | | URHD0017 | |
| | MeOXC4 | Methylorubrum | C5AX46 |
| | | extorquens | |
| | JGI15 | Alphaproteobacteria | A0A3C0TX30 |
| | | bacterium | |
| ACT2/TES1 | ydiF | Megasphaera | G0VND6 |
| | | elsdenii | |
| | tesA | Escherichia coli | P0ADA1 |
| HADH | ldh | Lacticaseibacillus | P00343 |
| | | casei | |
| | ldh | Desulfovibrio | A1VG02 |
| | | vulgaris | |
| AADH | ald | Bacillus subtilis | Q08352 |
| | pdh | Thermoactinomyces | P22823 |
| | | intermedius | |
| ACR2 | pduP | Salmonella | Q9XDN1 |
| | | typhimurium (strain | |
| | | LT2) | |
| ADH1 | yahK | Escherichia coli | P75691 |
| DDR1 | pduC | Salmonella | P37450 |
| | pduD | typhimurium (strain | O31041 |
| | pduE | LT2) | O31042 |
| | pddA | Klebsiella oxytoca | Q59470 |
| | pddB | | Q59471 |
| | pddC | | Q59472 |
| DDR2 | RiDD | Roseburia | Q1A666 |
| | | inulinivorans | |
| ADH2 | yahk | Escherichia coli | P75691 |
| HACD | lcdAB | Anaerotignum | G3KIM3 |
| | | propionicum | G3KIM4 |
| ACT3/TES2 | acuN | Halomonas sp. | C8YX88 |
| | | HTNK1 | |
| | gbuF | Emergencia | A0A415DZ10 |
| | | timonensis | |
| ECH | acuK | Halomonas sp. | C8YX87 |
| | | HTNK1 | |
| | crt | Clostridium | P52046 |
| | | acetobutylicum | |
| | lvaC | Pseudomonas | Q88J56 |
| | | putida KT2440 | |
| ACT4/TES3 | acuK | Halomonas sp. | C8YX87 |
| | | HTNK1 | |
| | tesB | Escherichia coli | P0AGG2 |
| TER | pcs | Chloroflexus | Q8VRG6 |
| | | aurantiacus | |
| | bcd | Clostridium kluyveri | A5N5C8 |
| | fadE | Escherichia coli | Q47146 |
| ACR3 | pduP | Salmonella | Q9XDN1 |
| | | typhimurium (strain | |
| | | LT2) | |
| | ald | Clostridium | Q716S8 |
| | | beijerinckii | |

### Example 15: Carboxylic Acid Platform Using Acetic Acid-Derived Acetaldehyde as Substrate for Condensation with Formyl-COA

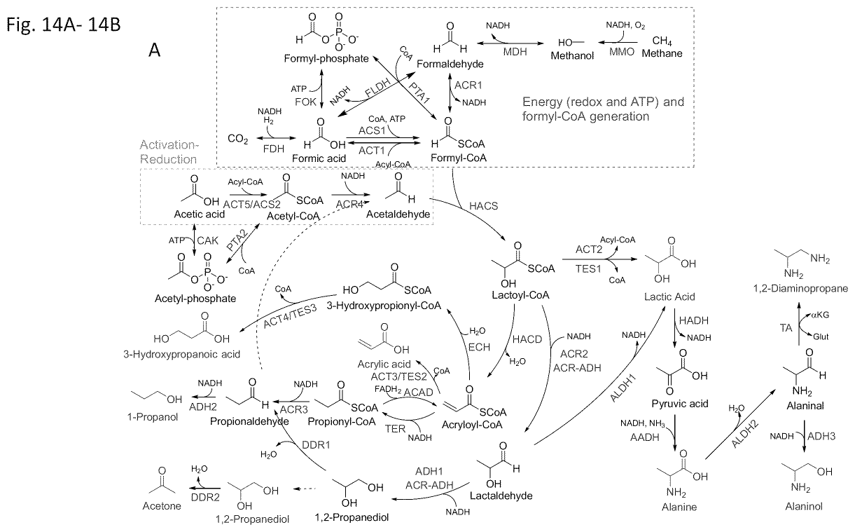

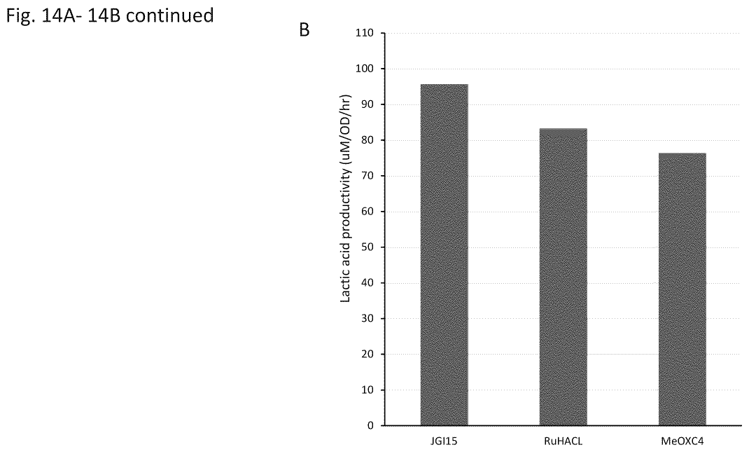

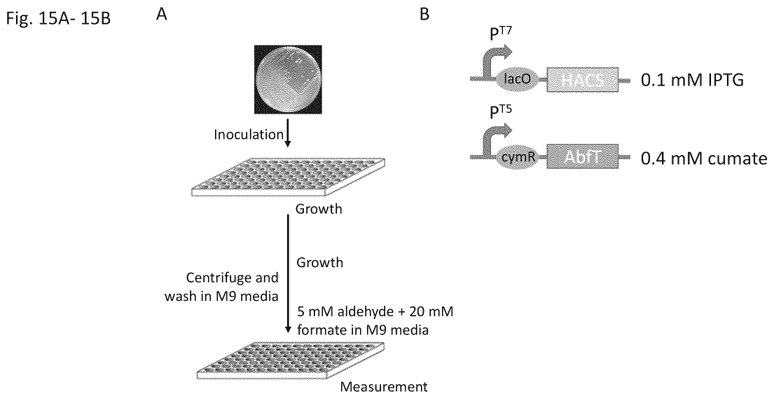

The purpose of this example is to demonstrate the implementation of the carboxylic acid (CA) platform using acetic acid (R═H) as the CA intermediate with C1 elongation pathways (FIG. 14A). Acetate can be supplied as the carbon source or generated from electrochemical reduction or microbial conversion from acetogenic bacteria using CO2 as the carbon source. Conversion of CO2 to acetate has been demonstrated using CO2 electrolyzer system with up to 57% carbon selectivity (Hann et al. Nat. Food 3:461-471 (2022)).

Activation of acetic acid to acetyl-CoA and then reduction to acetaldehyde have been well studied in the literatures. Acetic acid can be activated to acetyl-CoA through acetyl-phosphate. First, acetic acid is phosphorylated to acetyl phosphate by acetate kinase from Escherichia coli (Skarstedt et al. J Biol Chem. 251(21):6775-6783 (1976)) and then converted to acetyl-CoA by phosphate acetyltransferase from Escherichia coli (Campos-Bermudez et al. FEBS J. 277(8): 1957-1966 (2010)). The other pathway to activate acetic acid is acetyl-CoA synthetase from Escherichia coli (Biochem Biophys Res Commun. 449(3):272-277 (2014)) or succinyl-CoA transferase from Clostridium kluyveri (Sohling et al. Eur J Biochem. 212(1): 121-127 (1993)). Finally, acetyl-CoA is reduced to acetaldehyde by acetaldehyde dehydrogenase from Escherichia coli (Song et al. Metab Eng. 35:38-45 (2016)).

C1 elongation is initiated by formyl-CoA production through energy and formyl-CoA generation steps. 2-hydroxyacyl-CoA synthase (HACS) condenses acetaldehyde and formyl-CoA to produce lactoyl-CoA. Lactoyl-CoA is converted to lactic acid by lactoyl-CoA transferase from Megasphaera elsdenii (Niu et al. ACS Synth Biol. 4(4):378-382 (2015)).

Lactic acid can be further oxidized to pyruvic acid by lactate dehydrogenase from Lacticaseibacillus casei (Gordon et al. Eur J Biochem. 67(2):543-555 (1976)). Pyruvate is reduced to alanine by alanine dehydrogenase from Bacillus subtilis (Yoshida et al. Methods in enzymology 17:176-181 (1970)).

Lactoyl-CoA as the product of condensation can be reduced to 1,2-propanediol (1,2-PDO), acetone and 1-propanol. First, lactoyl-CoA is reduced to lactaldehyde through lactaldehyde dehydrogenase from Salmonella enterica (Niu et al. ACS Synth Biol. 4(4):378-382 (2015)) which can be further reduced to 1,2-PDO by lactaldehyde reductase from E. coli (Niu et al. ACS Synth Biol. 4(4):378-382 (2015)). Dehydration of 1,2-PDO by propanediol dehydratase from Roseburia inulinivorans (LaMattina et al. J Biol Chem. 291(30):15515-15526 (2016)) produces acetone. Moreover, dehydration of 1,2-PDO by diol dehydratase from Salmonella enterica (Bibok et al. J Bacteriol. 179(21):6633-6639(1997)) produces propionaldehyde. The resulting propionaldehyde can be fed to the subsequent iteration of C1 elongation or reduced to 1-propanol by aldehyde reductase from E. coli (Pick et al. Appl Microbiol Biotechnol. 97(13):5815-5824(2013)).

Lactoyl-CoA can be used to produce unsaturated acids. Lactoyl-CoA is dehydrated to acryloyl-CoA by lactoyl-CoA dehydratase from Anaerotignum propionicum (Kandasamy et al. Appl Microbiol Biotechnol. 97(3): 1191-1200(2013)). Acryloyl-CoA is converted to acrylic acid through CoA transferase from Halomonas sp. HTNK1 (Todd et al. Environ Microbiol. 12(2):327-343 (2010)). Moreover, acryloyl-CoA can be hydrolyzed to 3-hydroxypropionyl-CoA and further oxidized to 3-hydroxypropionic acid by acryloyl-CoA hydratase from Halomonas sp. HTNK1 (Todd et al. Environ Microbiol. 12(2):327-343 (2010)). Another pathway for acryloyl-CoA is to be converted to propionyl-CoA by propionyl-CoA synthase from Chloroflexus aurantiacus (Alber et al. J Biol Chem. 277(14): 12137-12143 (2002)). Propionyl-CoA is further reduced to propionaldehyde by propanal dehydrogenase from Salmonella enterica (Niu et al. ACS Synth Biol. 4(4):378-382 (2015)).

To implement the C1 elongation pathway for lactic acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1 (FIG. 15B). As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.2 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 5 mM acetaldehyde and 20 mM formate were added at 0 hr and were incubated at 30° C. and 1000 rpm in Digital Microplate Shaker (Fisher Scientific). After incubation at 30° C. for 1 hour, the cells were pelleted by centrifugation and the media was analyzed using HPLC (FIG. 15A).

The result indicates that BsmHACS is the best candidate under the given experimental conditions reaching up to 95 μM/hr per OD600 (FIG. 14B).

### Example 16: Carboxylic Acid Platform Using Propionic Acid-Derived Propionaldehyde as Substrate for Condensation with Formyl-COA

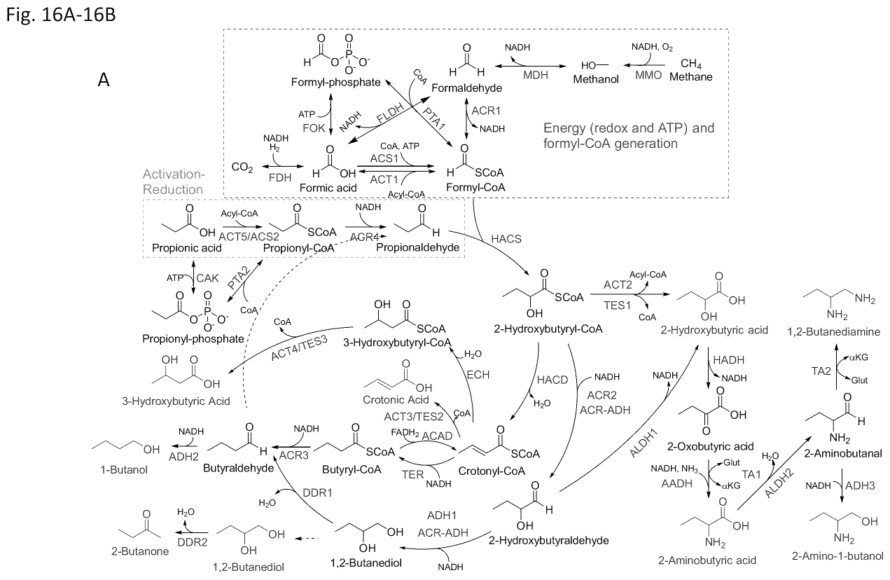

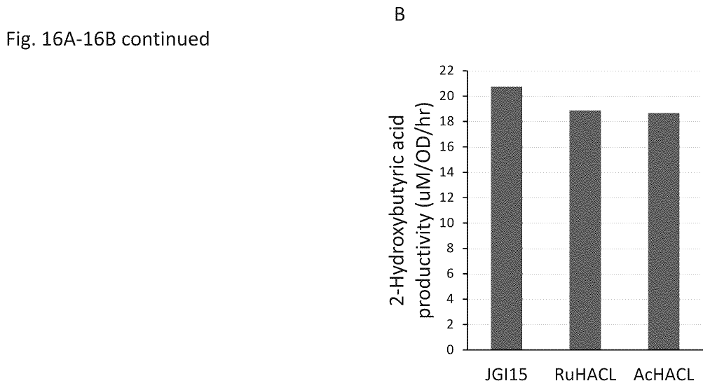

The purpose of this example is to demonstrate the implementation of the carboxylic acid (CA) platform using propionic acid (R═CH₃) as the CA intermediate with C1 elongation pathways (FIG. 16A).

Activation of propionic acid to propionyl-CoA and then reduction to propionaldehyde have been well studied in the literatures. Propionic acid can be activated to propionyl-CoA through propionyl-phosphate. First, propionic acid is phosphorylated to propionyl-phosphate by propionate kinase from Escherichia coli (Hesslinger et al. Mol Microbiol. 27(2):477-492(1998)) and then converted to propionyl-CoA by phosphate acetyltransferase from Escherichia coli (Hesslinger et al. Mol Microbiol. 27(2):477-492(1998)). The other pathway to activate propionic acid is propionyl-CoA synthetase from Escherichia coli (Guo et al. Prikl Biokhim Mikrobiol. 48(3):289-293(2012)) or succinyl-CoA transferase from Escherichia coli (Haller et al. Biochemistry. 39(16):4622-4629(2000)). Finally, propionyl-CoA is reduced to propionaldehyde by propanal dehydrogenase from Salmonella enterica (Niu et al. ACS Synth Biol. 4(4):378-382(2015)).

C1 elongation is initiated by formyl-CoA production through energy and formyl-CoA generation steps. 2-hydroxyacyl-CoA synthase (HACS) condenses propionaldehyde and formyl-CoA to produce 2-hydroxybutyryl-CoA. 2-Hydroxybutyryl-CoA is converted to 2-hydroxybutyric acid by acyl-CoA thioesterase from Escherichia coli (Lee et al. Biochem Biophys Res Commun. 231(2):452-456(1997)).

2-Hydroxybutyric acid can be further oxidized to 2-oxobutyric acid by lactate dehydrogenase from Desulfovibrio vulgaris (Ogata et al. J Biochem. 89(5):1423-1431 (1981)). 2-Oxobutyric acid is reduced to 2-aminobutyric acid by phenylalanine dehydrogenase from Thermoactinomyces intermedius (Kataoka et al. J Biochem. 116(4):931-936 (1994)).

2-Hydroxybutyryl-CoA as the product of condensation can be reduced to 1,2-butanediol (1,2-BDO), 2-butanone and 1-butanol. First, 2-hydroxybutyryl-CoA is reduced to 2-hydroxybutyraldehyde through aldehyde dehydrogenase from Salmonella enterica (Niu et al. ACS Synth Biol. 4(4):378-382 (2015)) which can be further reduced to 1,2-BDO by aldehyde reductase from E. coli (Niu et al. ACS Synth Biol. 4(4):378-382 (2015)). Dehydration of 1,2-BDO by diol dehydratase produces 2-butanone. Moreover, dehydration of 1,2-BDO by diol dehydratase from Klebsiella oxytoca (Toraya et al. J Biochem. 144(4):437-446 (2008)) produces butyraldehyde. The resulting butyraldehyde can be fed to the subsequent iteration of C1 elongation or reduced to 1-butanol by aldehyde reductase from E. coli (Pick et al. Appl Microbiol Biotechnol. 97(13):5815-5824(2013)).

2-Hydroxybutyryl-CoA can be used to produce unsaturated acids. 2-Hydroxybutyryl-CoA is dehydrated to crotonyl-CoA by acyl-CoA dehydratase from Anaerotignum propionicum (Kandasamy et al. Appl Microbiol Biotechnol. 97(3):1191-1200(2013)). Crotonyl-CoA is converted to crotonic acid through crotonyl-CoA thioesterase from Emergencia timonensis (Buffa et al. Nat Microbiol. 7(1):73-86 (2022)). Moreover, crotonyl-CoA can be hydrolyzed to 3-hydroxybutyryl-CoA by acyl-CoA dehydratase from Clostridium acetobutylicum (Waterson et al. Methods Enzymol. 71 Pt C:421-430 (1981)) and further oxidized to 3-hydroxybutyric acid by acyl-CoA thioesterase from E. coli (Tseng et al. Appl Environ Microbiol. 75(10):3137-3145 (2009)). Another pathway for crotonyl-CoA is to be converted to butyryl-CoA by butyryl-CoA dehydrogenase from Clostridium kluyveri (Li et al. J Bacteriol. 190(3):843-850 (2008)). Butyryl-CoA is further reduced to butyraldehyde by aldehyde dehydrogenase from Clostridium beijerinckii (Yan et al. Appl Environ Microbiol. 56(9):2591-2599 (1990)).

To implement the C1 elongation pathway for 2-hydroxybutyric acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1 (FIG. 15B). As a host for these vectors, we used an engineered strain of E. coli based on MG1655(DE3) with knockouts for formaldehyde (ΔfrmA) and formate (ΔfdhF-ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.2 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 5 mM propionaldehyde and 20 mM formate were added at 0 hr and were incubated at 30° C. and 1000 rpm in Digital Microplate Shaker (Fisher Scientific). After incubation at 30° C. for 1 hour, the cells were pelleted by centrifugation and the media was analyzed using HPLC (FIG. 15A).

The result indicates that BsmHACS is the best candidate under the given experimental conditions reaching up to 20 μM/hr per OD600 (FIG. 16B).

### Example 17: Carboxylic Acid Platform Using Butyric Acid-Derived Butyraldehyde as Substrate for Condensation with Formyl-COA

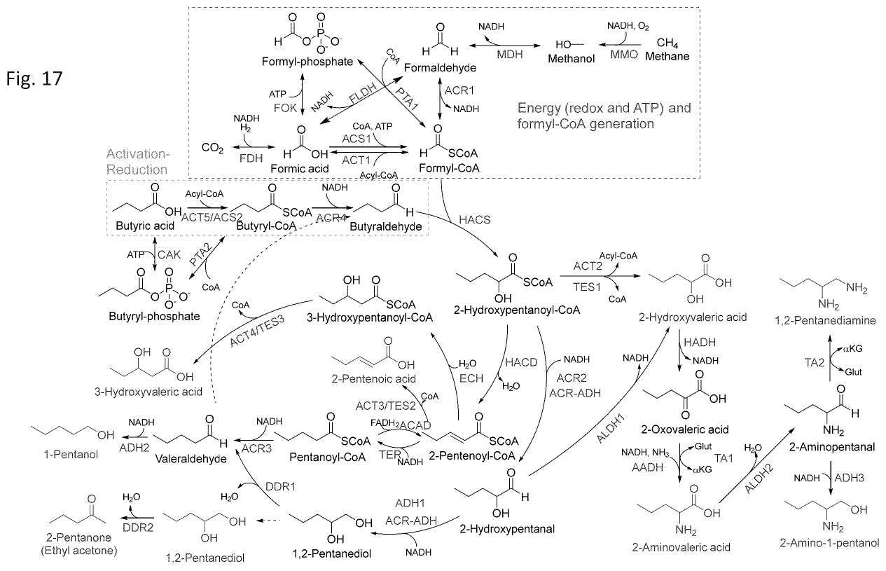

The purpose of this example is to demonstrate the implementation of the carboxylic acid (CA) platform using butyric acid (R═CH₂CH₃) as the CA intermediate with C1 elongation pathways (FIG. 17).

Activation of butyric acid to butyryl-CoA and then reduction to butyraldehyde have been well studied in the literatures. Butyric acid can be activated to butyryl-CoA through butyryl-phosphate. First, butyric acid is phosphorylated to butyryl-phosphate by butyrate kinase from Clostridium acetobutylicum (Cary et al. J Bacteriol. 170(10):4613-4618 (1988)) and then converted to butyryl-CoA by phosphotransbutyrylase from Clostridium acetobutylicum (Wiesenborn et al. Appl Environ Microbiol. 55(2):317-322 (1989)). The other pathway to activate butyric acid is CoA synthetase from Pseudomonas putida (Rand et al. Nat Microbiol. 2(12): 1624-1634 (2017)) or CoA transferase from Clostridium kluyveri (Seedorf et al. Proc Natl Acad Sci USA. 105(6):2128-2133 (2008)). Finally, butyryl-CoA is reduced to butyraldehyde by aldehyde dehydrogenase from Clostridium acetobutylicum (Fontaine et al. J Bacteriol. 184(3):821-830 (2002)).

C1 elongation is initiated by formyl-CoA production through energy and formyl-CoA generation steps. 2-hydroxyacyl-CoA synthase (HACS) condenses butyraldehyde and formyl-CoA to produce 2-hydroxypentanoyl-CoA. 2-Hydroxypentanoyl-CoA is converted to 2-hydroxyvaleric acid by acyl-CoA thioesterase. 2-Hydroxyvaleric acid can be further oxidized to 2-oxovaleric acid by 2-hydroxyacid dehydrogenase. 2-Oxovaleric acid is reduced to 2-aminovaleric acid by amino dehydrogenase/transaminase.

2-Hydroxypentanoyl-CoA as the product of condensation can be reduced to 1,2-pentanediol, 2-pentanone and valeraldehyde. First, 2-hydroxypentanoyl-CoA is reduced to 2-hydroxypentanal through aldehyde dehydrogenase which can be further reduced to 1,2-pentanediol by aldehyde reductase. Dehydration of 1,2-pentanediol by diol dehydratase produces 2-pentanone. Moreover, dehydration of 1,2-pentanediol by diol dehydratase produces valeraldehyde. The resulting valeraldehyde can be fed to the subsequent iteration of C1 elongation or reduced to 1-pentanol by aldehyde reductase.

2-Hydroxypentanoyl-CoA can be used to produce unsaturated acids. 2-Hydroxypentanoyl-CoA is dehydrated to 2-pentenoyl-CoA by acyl-CoA dehydratase. 2-Pentenoyl-CoA is converted to 2-pentenoic acid through CoA thioesterase. Moreover, 2-pentenoyl-CoA can be hydrolyzed to 3-hydroxypentanoyl-CoA by acyl-CoA dehydratase from Pseudomonas putida (Rand et al. Nat Microbiol. 2(12): 1624-1634 (2017)) and further oxidized to 3-hydroxyvaleric acid by acyl-CoA thioesterase. Another pathway for 2-pentenoyl-CoA is to be converted to pentanoyl-CoA by acyl-CoA dehydrogenase from E. coli (Campbell et al. J Bacteriol. 184(13):3759-3764 (2002)). Pentanoyl-CoA is further reduced to valeraldehyde by aldehyde dehydrogenase.

### Example 18: Carboxylic Acid Platform Using Glycolic Acid-Derived Glycolaldehyde as Substrate for Condensation with Formyl-COA

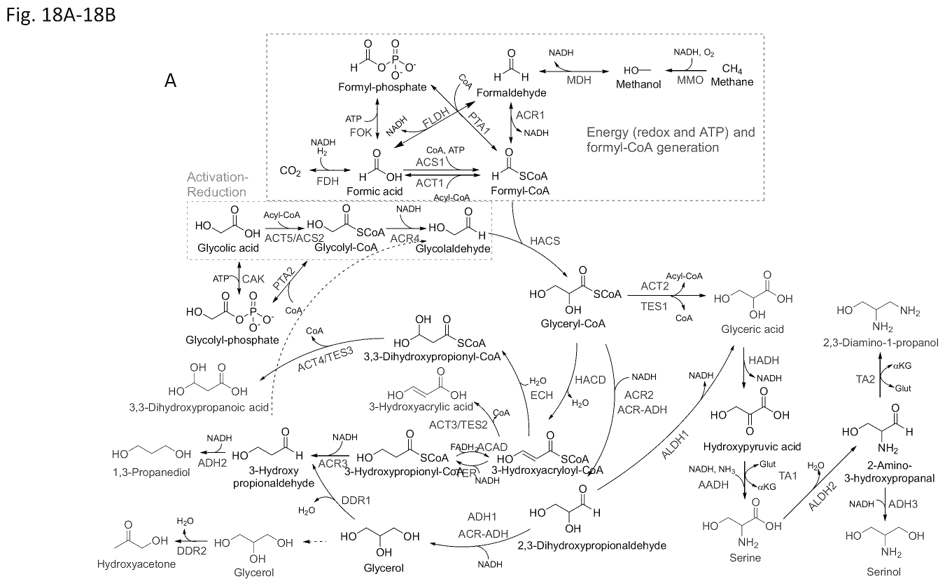

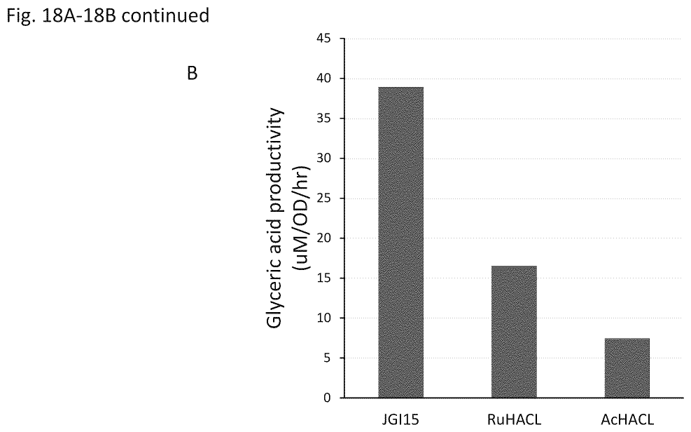

The purpose of this example is to demonstrate the implementation of the carboxylic acid (CA) platform using glycolic acid (R═OH) as the CA intermediate with C1 elongation pathways (FIG. 18A).

Glycolic acid can be activated to glycolyl-CoA through glycolyl-phosphate. First, glycolic acid is phosphorylated to glycolyl-phosphate by kinase and then converted to glycolyl-CoA by phosphotransacylase. The other pathway to activate glycolic acid is CoA synthetase from or CoA transferase. Finally, glycolyl-CoA is reduced to glycolaldehyde by aldehyde dehydrogenase.

C1 elongation is initiated by formyl-CoA production through energy and formyl-CoA generation steps. 2-hydroxyacyl-CoA synthase (HACS) condenses glycolaldehyde and formyl-CoA to produce glyceryl-CoA. Glyceryl-CoA is converted to glyceric acid by acyl-CoA thioesterase.

Glyceric acid can be further oxidized to hydroxypyruvic acid by 2-hydroxyacid dehydrogenase. Hydroxypyruvic acid is reduced to serine by amino dehydrogenase/transaminase. Dehydration of serine produces 2-amino-3-hydroxypropanal which can be further reduced to serinol or aminated to 2,3-diamino-1-propanol.

Glyceryl-CoA as the product of condensation can be reduced to glycerol, hydroxyacetone and 1,3-propanediol. First, glyceryl-CoA is reduced to 2,3-dihydroxypropionaldehyde through aldehyde dehydrogenase which can be further reduced to glycerol by aldehyde reductase. Dehydration of glycerol by diol dehydratase produces hydroxyacetone. Moreover, dehydration of glycerol by diol dehydratase produces 3-hydroxypropionaldehyde. The resulting 3-hydroxypropionaldehyde can be fed to the subsequent iteration of C1 elongation or reduced to 1,3-propanediol by aldehyde reductase.

Glyceryl-CoA can be used to produce unsaturated acids. Glyceryl-CoA is dehydrated to 3-hydroxyacryloyl-CoA by acyl-CoA dehydratase. 3-Hydroxyacryloyl-CoA is converted to 3-hydroxyacrylic acid through CoA thioesterase. Moreover, 3-hydroxyacryloyl-CoA can be hydrolyzed to 3,3-dihydroxypropionyl-CoA by acyl-CoA dehydratase and further oxidized to 3,3-dihydroxypropionic acid by acyl-CoA thioesterase. Another pathway for 3-hydroxyacryloyl-CoA is to be converted to 3-hydroxypropionyl-CoA by acyl-CoA dehydrogenase. 3-Hydroxypropionyl-CoA is further reduced to 3-hydroxypropionaldehyde by aldehyde dehydrogenase.

To implement the C1 elongation pathway for glyceric acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1 (FIG. 15B). As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.2 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 μg/mL spectinomycin) were included when appropriate.

Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 5 mM glycolaldehyde and 20 mM formate were added at 0 hr and were incubated at 30° C. and 1000 rpm in Digital Microplate Shaker (Fisher Scientific). After incubation at 30° C. for 1 hour, the cells were pelleted by centrifugation and the media was analyzed using HPLC (FIG. 15A).

The result indicates that JG115 is the best candidate under the given experimental conditions reaching up to 39 μM/hr per OD600 (FIG. 18B).

### Example 19: Carboxylic Acid Platform Using Lactic Acid-Derived Lactaldehyde as Substrate for Condensation with Formyl-COA

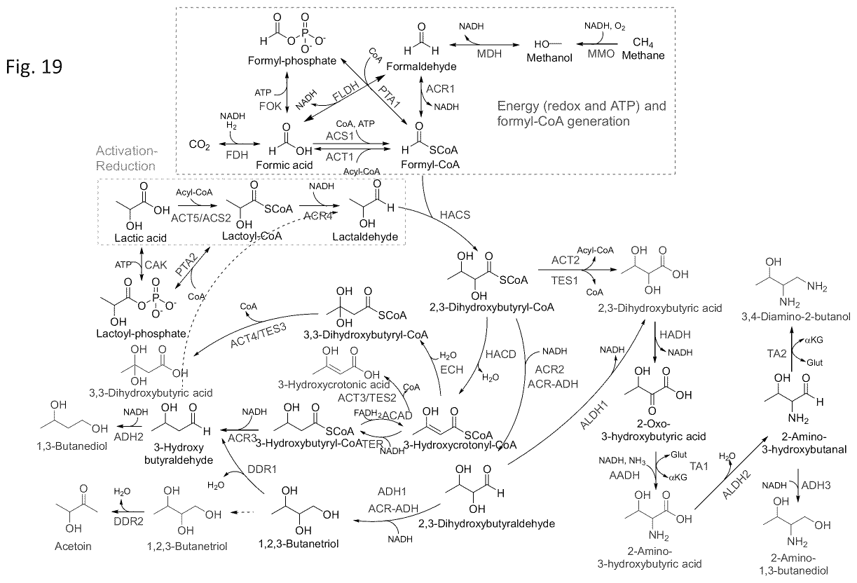

The purpose of this example is to demonstrate the implementation of the carboxylic acid (CA) platform using lactic acid (R═CH₃OH) as the CA intermediate with C1 elongation pathways (FIG. 19).

Lactic acid can be activated to lactoyl-CoA through lactoyl-phosphate. First, lactic acid is phosphorylated to lactoyl-phosphate by kinase and then converted to lactoyl-CoA by phosphotransacylase. The other pathway to activate lactic acid is CoA synthetase from or CoA transferase from Megasphaera elsdenii (Niu et al. ACS Synth Biol. 4(4):378-382 (2015)). Finally, lactoyl-CoA is reduced to lactaldehyde by aldehyde dehydrogenase from Salmonella enterica (Niu et al. ACS Synth Biol. 4(4):378-382 (2015)).

C1 elongation is initiated by formyl-CoA production through energy and formyl-CoA generation steps. 2-Hydroxyacyl-CoA synthase (HACS) condenses lactaldehyde and formyl-CoA to produce 2,3-dihydroxybutyryl-CoA. 2,3-Dihydroxybutyryl-CoA is converted to 2,3-dihydroxybutyric acid by acyl-CoA thioesterase.

2,3-Dihydroxybutyric acid can be further oxidized to 2-oxo-3-hydroxybutyric acid by 2-hydroxyacid dehydrogenase. 2-Oxo-3-hydroxybutyric acid is reduced to 2-amino-3-hydroxybutyric acid by amino dehydrogenase/transaminase. Dehydration of 2-amino-3-hydroxybutyric acid produces 2-amino-3-hydroxybutanal which can be further reduced to 2-amino-1,3-butanediol or aminated to 3,4-diamino-2-butanol.

2,3-Dihydroxybutyryl-CoA as the product of condensation can be reduced to 1,2,3-butanetriol, acetion and 1,3-butanediol. First, 2,3-dihydroxybutyryl-CoA is reduced to 2,3-dihydroxybutyraldehyde through aldehyde dehydrogenase which can be further reduced to 1,2,3-butanetriol by aldehyde reductase. Dehydration of 1,2,3-butanetriol by diol dehydratase produces acetion. Moreover, dehydration of 1,2,3-butanetriol by diol dehydratase produces 3-hydroxybutyraldehyde. The resulting 3-hydroxybutyraldehyde can be fed to the subsequent iteration of C1 elongation or reduced to 1,3-butanediol by aldehyde reductase.

2,3-Dihydroxybutyryl-CoA can be used to produce unsaturated acids. 2,3-Dihydroxybutyryl-CoA is dehydrated to 3-hydroxycrotonyl-CoA by acyl-CoA dehydratase. 3-Hydroxycrotonyl-CoA is converted to 3-hydroxycrotonic acid through CoA thioesterase. Moreover, 3-hydroxycrotonyl-CoA can be hydrolyzed to 3,3-dihydroxybutyryl-CoA by acyl-CoA dehydratase and further oxidized to 3,3-dihydroxybutyric acid by acyl-CoA thioesterase. Another pathway for 3-hydroxycrotonyl-CoA is to be converted to 3-hydroxybutyryl-CoA by acyl-CoA dehydrogenase. 3-Hydroxybutyryl-CoA is further reduced to 3-hydroxybutyraldehyde by aldehyde dehydrogenase.

### Example 20: Carboxylic Acid Platform Using Glyceric Acid-Derived Glyceraldehyde as Substrate for Condensation with Formyl-COA

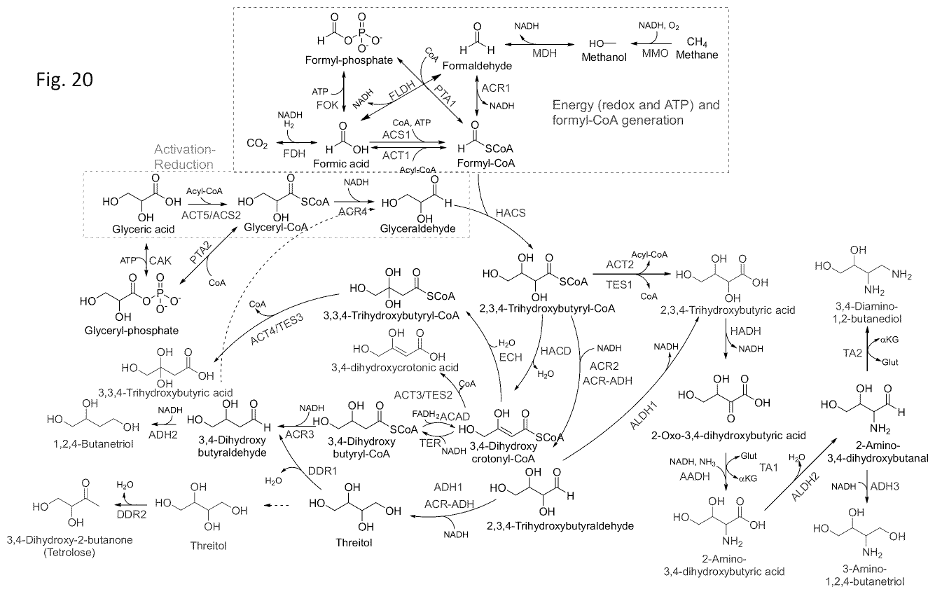

The purpose of this example is to demonstrate the implementation of the carboxylic acid (CA) platform using glyceric acid (R═OHCH₂OH) as the CA intermediate with C1 elongation pathways (FIG. 20).

Glyceric acid can be activated to glyceryl-CoA through glyceryl-phosphate. First, glyceric acid is phosphorylated to glyceryl-phosphate by kinase and then converted to glyceryl-CoA by phosphotransacylase. The other pathway to activate glyceric acid is CoA synthetase or CoA transferase. Finally, glyceryl-CoA is reduced to glyceraldehyde by aldehyde dehydrogenase.

C1 elongation is initiated by formyl-CoA production through energy and formyl-CoA generation steps. 2-Hydroxyacyl-CoA synthase (HACS) condenses glyceraldehyde and formyl-CoA to produce 2,3,4-trihydroxybutyryl-CoA. 2,3,4-Trihydroxybutyryl-CoA is converted to 2,3,4-trihydroxybutyric acid by acyl-CoA thioesterase.

2,3,4-Trihydroxybutyric acid can be further oxidized to 2-oxo-3,4-dihydroxybutyric acid by 2-hydroxyacid dehydrogenase. 2-Oxo-3,4-dihydroxybutyric acid is reduced to 2-amino-3,4-dihydroxybutyric acid by amino dehydrogenase/transaminase. Dehydration of 2-amino-3,4-dihydroxybutyric acid produces 2-amino-3,4-dihydroxybutanal which can be further reduced to 3-amino-1,2,4-butanetriol or aminated to 3,4-diamino-1,2-butanediol.

2,3,4-Trihydroxybutyryl-CoA as the product of condensation can be reduced to threitol, 3,4-dihydroxy-2-butanone and 1,2,4-butanetriol. First, 2,3,4-trihydroxybutyryl-CoA is reduced to 2,3,4-trihydroxybutyraldehyde through aldehyde dehydrogenase which can be further reduced to threitol by aldehyde reductase. Dehydration of threitol by diol dehydratase produces 3,4-dihydroxy-2-butanone. Moreover, dehydration of threitol by diol dehydratase produces 3,4-dihydroxybutyraldehyde. The resulting 3,4-dihydroxybutyraldehyde can be fed to the subsequent iteration of C1 elongation or reduced to 1,2,4-butanetriol by aldehyde reductase.

2,3,4-Trihydroxybutyryl-CoA can be used to produce unsaturated acids. 2,3,4-Trihydroxybutyryl-CoA is dehydrated to 3,4-dihydroxycrotonyl-CoA by acyl-CoA dehydratase. 3,4-Dihydroxycrotonyl-CoA is converted to 3,4-dihydroxycrotonic acid through CoA thioesterase. Moreover, 3,4-dihydroxycrotonyl-CoA can be hydrolyzed to 3,3,4-trihydroxybutyryl-CoA by acyl-CoA dehydratase and further oxidized to 3,3,4-trihydroxybutyric acid by acyl-CoA thioesterase. Another pathway for 3,4-dihydroxycrotonyl-CoA is to be converted to 3,4-dihydroxybutyryl-CoA by acyl-CoA dehydrogenase. 3,4-Dihydroxybutyryl-CoA is further reduced to 3,4-dihydroxybutyraldehyde by aldehyde dehydrogenase.

### Example 21: Carboxylic Acid Platform Using 3-Hydroxypropionic Acid-Derived 3-Hydroxypropionaldehyde as Substrate for Condensation with Formyl-COA

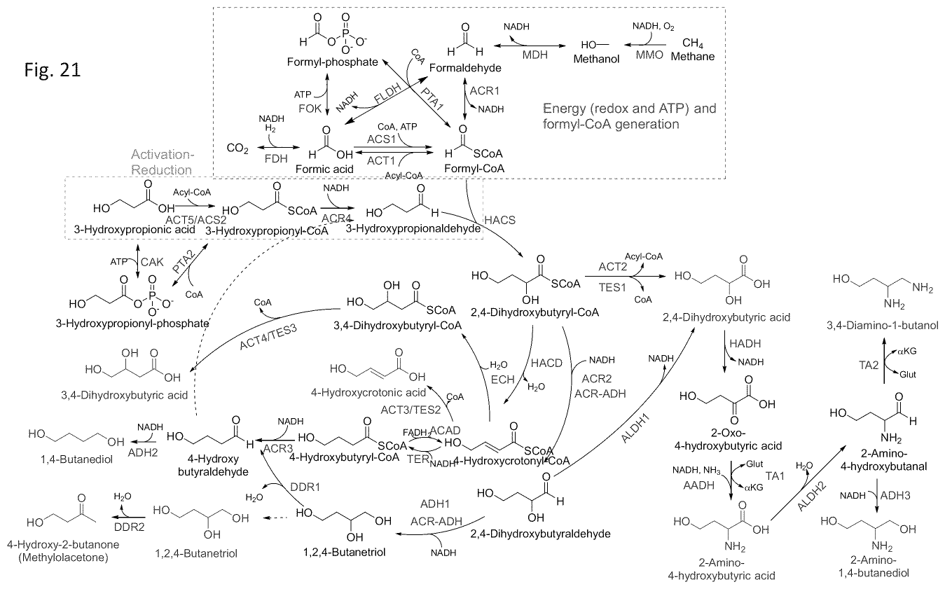

The purpose of this example is to demonstrate the implementation of the carboxylic acid (CA) platform using 3-hydroxypropionic acid (R═CH₂OH) as the CA intermediate with C1 elongation pathways (FIG. 21).

3-Hydroxypropionic acid can be activated to 3-hydroxypropionyl-CoA through 3-hydroxypropionyl-phosphate. First, 3-hydroxypropionic acid is phosphorylated to 3-hydroxypropionyl-phosphate by kinase and then converted to 3-hydroxypropionyl-CoA by phosphotransacylase. The other pathway to activate 3-hydroxypropionic acid is CoA synthetase or CoA transferase. Finally, 3-hydroxypropionyl-CoA is reduced to 3-hydroxypropionaldehyde by aldehyde dehydrogenase.

C1 elongation is initiated by formyl-CoA production through energy and formyl-CoA generation steps. 2-Hydroxyacyl-CoA synthase (HACS) condenses 3-hydroxypropionaldehyde and formyl-CoA to produce 2,4-dihydroxybutyryl-CoA. 2,4-Dihydroxybutyryl-CoA is converted to 2,4-dihydroxybutyric acid by acyl-CoA thioesterase.

2,4-Dihydroxybutyric acid can be further oxidized to 2-oxo-4-hydroxybutyric acid by 2-hydroxyacid dehydrogenase. 2-Oxo-4-hydroxybutyric acid is reduced to 2-amino-4-hydroxybutyric acid by amino dehydrogenase/transaminase. Dehydration of 2-amino-4-hydroxybutyric acid produces 2-amino-4-hydroxybutanal which can be further reduced to 2-amino-1,4-butanediol or aminated to 3,4-diamino-1-butanol.

2,4-Dihydroxybutyryl-CoA as the product of condensation can be reduced to 1,2,4-butanetriol, 4-hydroxy-2-butanone and 1,4-butanediol. First, 2,4-dihydroxybutyryl-CoA is reduced to 2,4-dihydroxybutyraldehyde through aldehyde dehydrogenase which can be further reduced to 1,2,4-butanetriol by aldehyde reductase. Dehydration of 1,2,4-butanetriol by diol dehydratase produces 4-hydroxy-2-butanone. Moreover, dehydration of 1,2,4-butanetriol by diol dehydratase produces 4-hydroxybutyraldehyde. The resulting 4-hydroxybutyraldehyde can be fed to the subsequent iteration of C1 elongation or reduced to 1,4-butanediol by aldehyde reductase.

2,4-Dihydroxybutyryl-CoA can be used to produce unsaturated acids. 2,4-Dihydroxybutyryl-CoA is dehydrated to 4-hydroxycrotonyl-CoA by acyl-CoA dehydratase. 4-Hydroxycrotonyl-CoA is converted to 4-hydroxycrotonic acid through CoA thioesterase. Moreover, 4-hydroxycrotonyl-CoA can be hydrolyzed to 3,4-dihydroxybutyryl-CoA by acyl-CoA dehydratase and further oxidized to 3,4-dihydroxybutyric acid by acyl-CoA thioesterase. Another pathway for 4-hydroxycrotonyl-CoA is to be converted to 4-hydroxybutyryl-CoA by acyl-CoA dehydrogenase. 4-Hydroxybutyryl-CoA is further reduced to 4-hydroxybutyraldehyde by aldehyde dehydrogenase.

### Example 22: Carboxylic Acid Platform Using Oxalic Acid-Derived Oxalic Semialdehyde as Substrate for Condensation with Formyl-COA

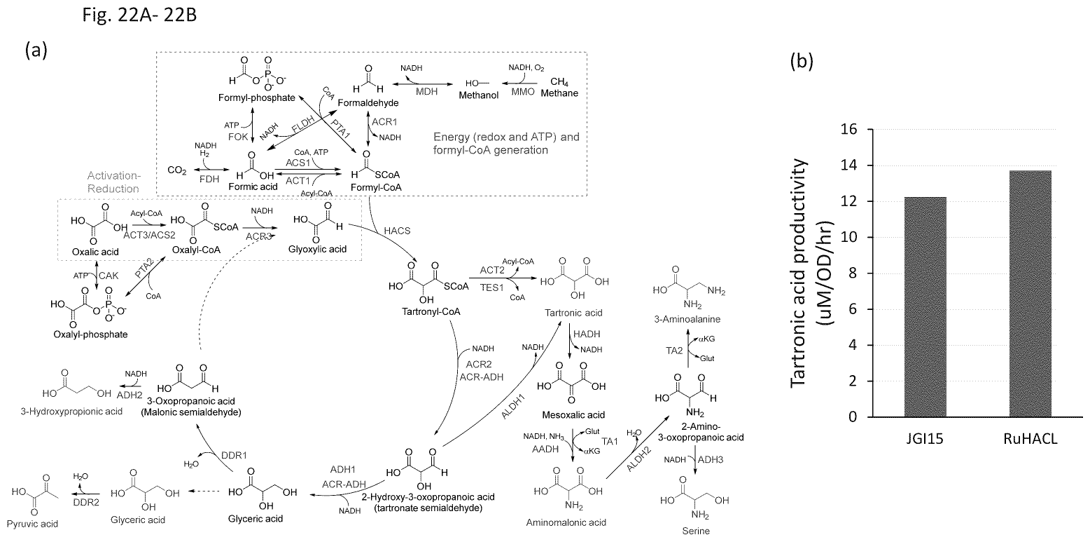

The purpose of this example is to demonstrate the implementation of the carboxylic acid (CA) platform using oxalic acid (R═OOH) as the CA intermediate with C1 elongation pathways (FIG. 22A).

Oxalic acid can be activated to oxalyl-CoA through oxalyl-phosphate. First, oxalic acid is phosphorylated to oxalyl-phosphate by kinase and then converted to oxalyl-CoA by phosphotransacylase. The other pathway to activate oxalic acid is oxalate-CoA ligase from Arabidopsis thaliana (Foster et al. Plant Cell. 24(3):1217-1229 (2012)) or CoA transferase from E. coli (Mullins et al. PLOS One. 8(7):e67901 (2013)). Finally, oxalyl-CoA is reduced to glyoxylic acid by aldehyde dehydrogenase.

C1 elongation is initiated by formyl-CoA production through energy and formyl-CoA generation steps. 2-Hydroxyacyl-CoA synthase (HACS) condenses glyoxylic acid and formyl-CoA to produce tartronyl-CoA. Tartronyl-CoA is converted to tartronic acid by acyl-CoA thioesterase. Tartronic acid can be further oxidized to mesoxalic acid by 2-hydroxyacid dehydrogenase. Mesoxalic acid is reduced to aspartic acid by amino dehydrogenase/transaminase. Dehydration of Aminomalonic acid produces 2-amino-3-oxopropionic acid which can be further reduced to serine or aminated to 3-aminoalanine.

Tartronyl-CoA as the product of condensation can be reduced to glyceric acid, pyruvic acid and 3-hydroxypropionic acid. First, tartronyl-CoA is reduced to 2-hydroxy-3-oxopropionic acid through aldehyde dehydrogenase which can be further reduced to glyceric acid by aldehyde reductase. Dehydration of glyceric acid by diol dehydratase produces pyruvic acid. Moreover, dehydration of glyceric acid by diol dehydratase produces 3-oxopropionic acid. The resulting 3-oxopropionic acid can be fed to the subsequent iteration of C1 elongation or reduced to 3-hydroxypropionic acid by aldehyde reductase.

To implement the C1 elongation pathway for tartronic acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1 (FIG. 15B). As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.2 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 5 mM glyoxylic acid and 20 mM formate were added at 0 hr and were incubated at 30° C. and 1000 rpm in Digital Microplate Shaker (Fisher Scientific). After incubation at 30° C. for 1 hour, the cells were pelleted by centrifugation and the media was analyzed using HPLC (FIG. 15A).

The result indicates that RuHACL is the best candidate under the given experimental conditions reaching up to 13 μM/hr per OD600 (FIG. 22B).

### Example 23: Carboxylic Acid Platform Using Malonic Acid-Derived Malonic Semialdehyde as Substrate for Condensation with Formyl-COA

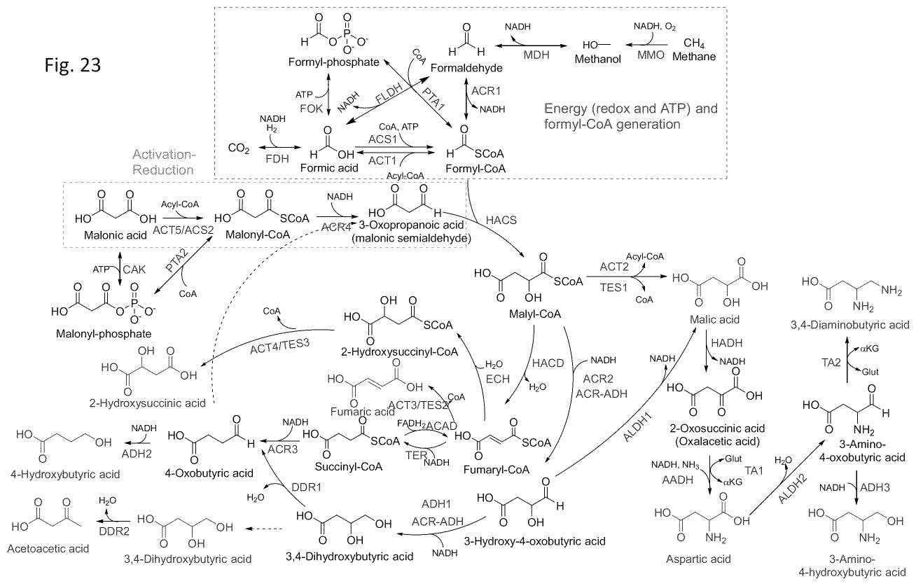

The purpose of this example is to demonstrate the implementation of the carboxylic acid (CA) platform using malonic acid (R═COOH) as the CA intermediate with C1 elongation pathways (FIG. 23).

Malonic acid can be activated to malonyl-CoA through malonyl-phosphate. First, malonic acid is phosphorylated to malonyl-phosphate by kinase and then converted to malonyl-CoA by phosphotransacylase. The other pathway to activate malonic acid is CoA synthetase or CoA transferase. Finally, malonyl-CoA is reduced to malonic semialdehyde by aldehyde dehydrogenase.

C1 elongation is initiated by formyl-CoA production through energy and formyl-CoA generation steps. 2-Hydroxyacyl-CoA synthase (HACS) condenses malonic semialdehyde and formyl-CoA to produce malyl-CoA. Malyl-CoA is converted to malic acid by acyl-CoA thioesterase.

Malic acid can be further oxidized to 2-oxosuccinic acid (oxalacetic acid) by 2-hydroxyacid dehydrogenase. 2-Oxosuccinic acid is reduced to aspartic acid by amino dehydrogenase/transaminase. Dehydration of aspartic acid produces 3-amino-4-oxobutyric acid which can be further reduced to 3-amino-4-hydroxybutyric acid or aminated to 3,4-diaminobutyric acid.

Malyl-CoA as the product of condensation can be reduced to 3,4-dihydroxybutyric acid, acetoacetic acid and 4-hydroxybutyric acid. First, malyl-CoA is reduced to 3-hydroxy-4-oxobutyric acid through aldehyde dehydrogenase which can be further reduced to 3,4-dihydroxybutyric acid by aldehyde reductase. Dehydration of 3,4-dihydroxybutyric acid by diol dehydratase produces acetoacetic acid. Moreover, dehydration of 3,4-dihydroxybutyric acid by diol dehydratase produces 4-oxobutyric acid. The resulting 4-oxobutyric acid can be fed to the subsequent iteration of C1 elongation or reduced to 4-hydroxybutyric acid by aldehyde reductase.

Malyl-CoA can be used to produce unsaturated acids. Malyl-CoA is dehydrated to fumaryl-CoA by acyl-CoA dehydratase. Fumaryl-CoA is converted to fumaric acid through CoA thioesterase. Moreover, fumaryl-CoA can be hydrolyzed to 2-hydroxysuccinyl-CoA by acyl-CoA dehydratase and further oxidized to 2-hydroxysuccinic acid by acyl-CoA thioesterase. Another pathway for fumaryl-CoA is to be converted to succinyl-CoA by acyl-CoA dehydrogenase. Succinyl-CoA is further reduced to 4-oxobutyric acid by aldehyde dehydrogenase.

### Example 24: Carboxylic Acid Platform Using Succinic Acid-Derived Succinic Semialdehyde as Substrate for Condensation with Formyl-COA

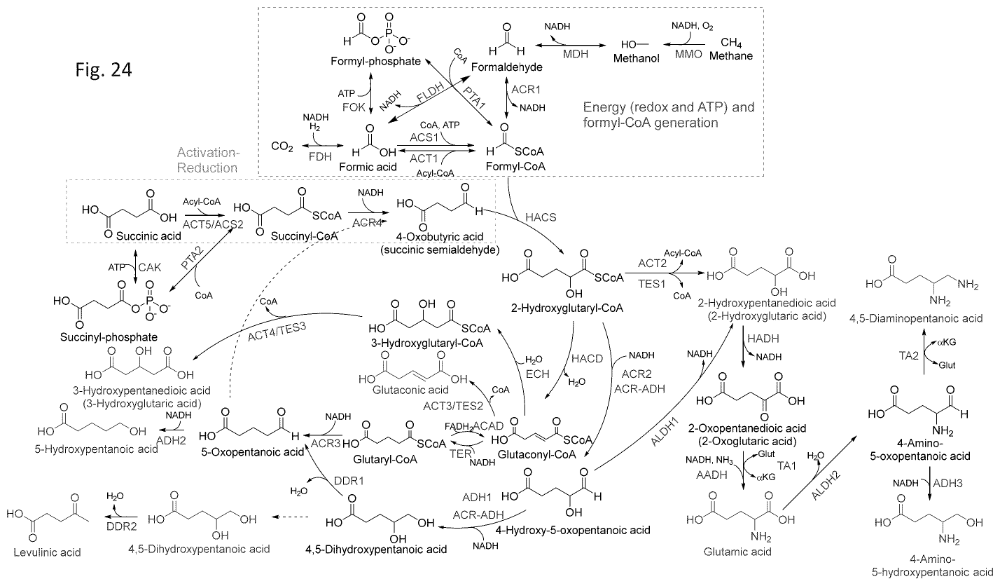

The purpose of this example is to demonstrate the implementation of the carboxylic acid (CA) platform using succinic acid (R═CH₂COOH) as the CA intermediate with C1 elongation pathways (FIG. 24).

Activation of succinic acid to succinyl-CoA and then reduction to succinic semialdehyde have been well studied in the literatures. Succinic acid can be activated to succinyl-CoA through succinyl-phosphate. First, succinic acid is phosphorylated to succinyl-phosphate by kinase and then converted to succinyl-CoA by phosphotransacylase. The other pathway to activate succinic acid is CoA synthetase from E. coli (Yim et al. Nat Chem Biol. 7(7):445-452 (2011)) or CoA transferase. Finally, succinyl-CoA is reduced to succinic semialdehyde by aldehyde dehydrogenase from Clostridium kluyveri (Schwander et al. Science. 354(6314):900-904 (2016)).

C1 elongation is initiated by formyl-CoA production through energy and formyl-CoA generation steps. 2-Hydroxyacyl-CoA synthase (HACS) condenses succinic semialdehyde and formyl-CoA to produce 2-hydroxyglutaryl-CoA. 2-Hydroxyglutaryl-CoA is converted to 2-hydroxyglutaric acid by acyl-CoA thioesterase.

2-Hydroxyglutaric acid can be further oxidized to 2-oxoglutaric acid by 2-hydroxyacid dehydrogenase. 2-Oxoglutaric acid is reduced to glutamic acid by amino dehydrogenase/transaminase. Dehydration of glutamic acid produces 4-amino-5-oxopentanoic acid which can be further reduced to 4-amino-5-hydroxypentanoic acid or aminated to 4,5-diaminopentanoic acid.

2-Hydroxyglutaryl-CoA as the product of condensation can be reduced to 4,5-dihydroxypentanoic acid, levulinic acid and 5-hydroxypentanoic acid. First, 2-hydroxyglutaryl-CoA is reduced to 4-hydroxy-5-oxopentanoic acid through aldehyde dehydrogenase which can be further reduced to 4,5-dihydroxypentanoic acid by aldehyde reductase. Dehydration of 4,5-dihydroxypentanoic acid by diol dehydratase produces levulinic acid. Moreover, dehydration of 4,5-dihydroxypentanoic acid by diol dehydratase produces 5-oxopentanoic acid. The resulting 5-oxopentanoic acid can be fed to the subsequent iteration of C1 elongation or reduced to 5-hydroxypentanoic acid by aldehyde reductase.

2-Hydroxyglutaryl-CoA can be used to produce unsaturated acids. 2-Hydroxyglutaryl-CoA is dehydrated to glutaconyl-CoA by acyl-CoA dehydratase. Glutaconyl-CoA is converted to glutaconic acid through CoA thioesterase. Moreover, glutaconyl-CoA can be hydrolyzed to 3-hydroxyglutaryl-CoA by acyl-CoA dehydratase and further oxidized to 3-hydroxyglutaric acid by acyl-CoA thioesterase. Another pathway for glutaconyl-CoA is to be converted to glutaryl-CoA by acyl-CoA dehydrogenase. Glutaryl-CoA is further reduced to 5-oxopentanoic acid by aldehyde dehydrogenase.

### Example 25: Carboxylic Acid Platform Using Isobutyric Acid-Derived Isobutyraldehyde as Substrate for Condensation with Formyl-COA

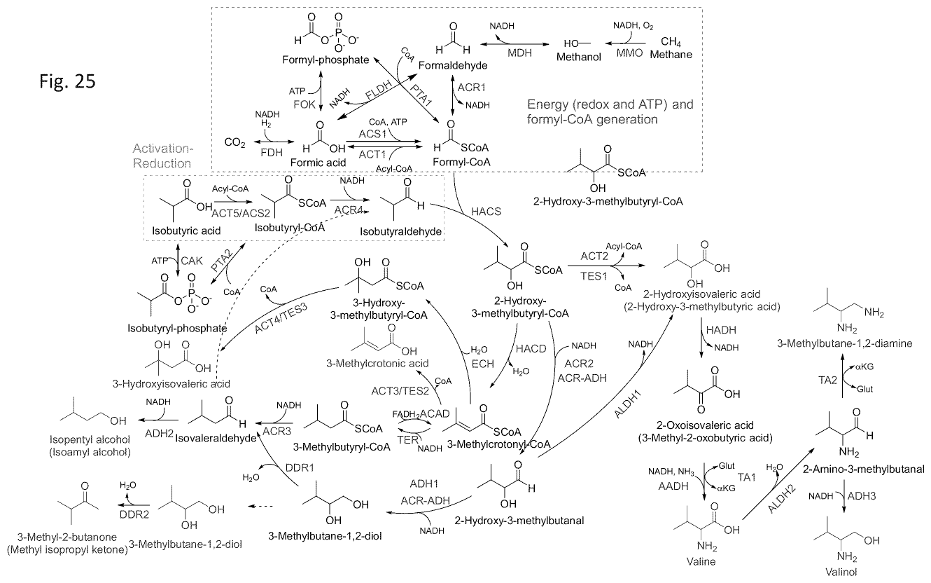

The purpose of this example is to demonstrate the implementation of the carboxylic acid (CA) platform using isobutyric acid (R═CH₃CH₃) as the CA intermediate with C1 elongation pathways (FIG. 25).

Isobutyric acid can be activated to isobutyryl-CoA through isobutyryl-phosphate. First, isobutyric acid is phosphorylated to isobutyryl-phosphate by kinase and then converted to isobutyryl-CoA by phosphotransacylase. The other pathway to activate isobutyric acid is CoA synthetase or CoA transferase. Finally, isobutyryl-CoA is reduced to isobutyraldehyde by aldehyde dehydrogenase.

C1 elongation is initiated by formyl-CoA production through energy and formyl-CoA generation steps. 2-Hydroxyacyl-CoA synthase (HACS) condenses isobutyraldehyde and formyl-CoA to produce 2-hydroxy-3-methylbutyryl-CoA. 2-Hydroxy-3-methylbutyryl-CoA is converted to 2-hydroxyisovaleric acid by acyl-CoA thioesterase.

2-Hydroxyisovaleric acid can be further oxidized to 2-oxoisovaleric acid by 2-hydroxyacid dehydrogenase. 2-Oxoisovaleric acid is reduced to valine by amino dehydrogenase/transaminase. Dehydration of valine produces 2-amino-3-methylbutanal which can be further reduced to valinol or aminated to 3-methylbutane-1,2-diamine.

2-Hydroxy-3-methylbutyryl-CoA as the product of condensation can be reduced to 3-methylbutane-1,2-diol, 3-methyl-2-butanone and isoamyl alcohol. First, 2-hydroxy-3-methylbutyryl-CoA is reduced to 2-hydroxy-3-methylbutanal through aldehyde dehydrogenase which can be further reduced to 3-methylbutane-1,2-diol by aldehyde reductase. Dehydration of 3-methylbutane-1,2-diol by diol dehydratase produces 3-methyl-2-butanone. Moreover, dehydration of 3-methylbutane-1,2-diol by diol dehydratase produces isovaleraldehyde. The resulting isovaleraldehyde can be fed to the subsequent iteration of C1 elongation or reduced to isoamyl alcohol by aldehyde reductase.

2-Hydroxy-3-methylbutyryl-CoA can be used to produce unsaturated acids. 2-Hydroxy-3-methylbutyryl-CoA is dehydrated to 3-methylcrotonyl-CoA by acyl-CoA dehydratase. 3-Methylcrotonyl-CoA is converted to 3-methylcrotonic acid through CoA thioesterase. Moreover, 3-methylcrotonyl-CoA can be hydrolyzed to 3-hydroxy-3-methylbutyryl-CoA by acyl-CoA dehydratase and further oxidized to 3-hydroxyisovaleric acid by acyl-CoA thioesterase. Another pathway for 3-methylcrotonyl-CoA is to be converted to 3-methylbutyryl-CoA by acyl-CoA dehydrogenase. 3-Methylbutyryl-CoA is further reduced to isovaleraldehyde by aldehyde dehydrogenase.

### Example 26: Carboxylic Acid Platform Using Isovaleric Acid-Derived Isovaleraldehyde as Substrate for Condensation with Formyl-COA

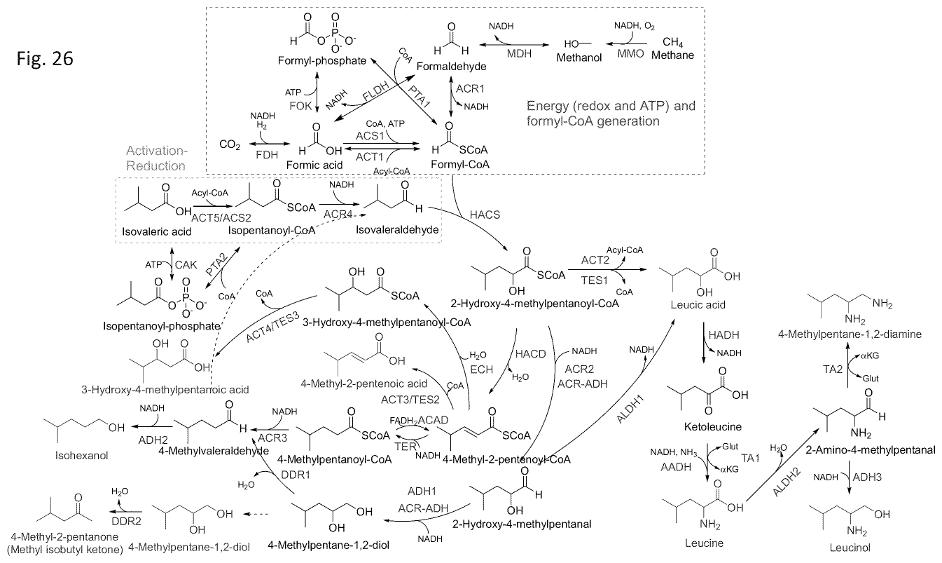

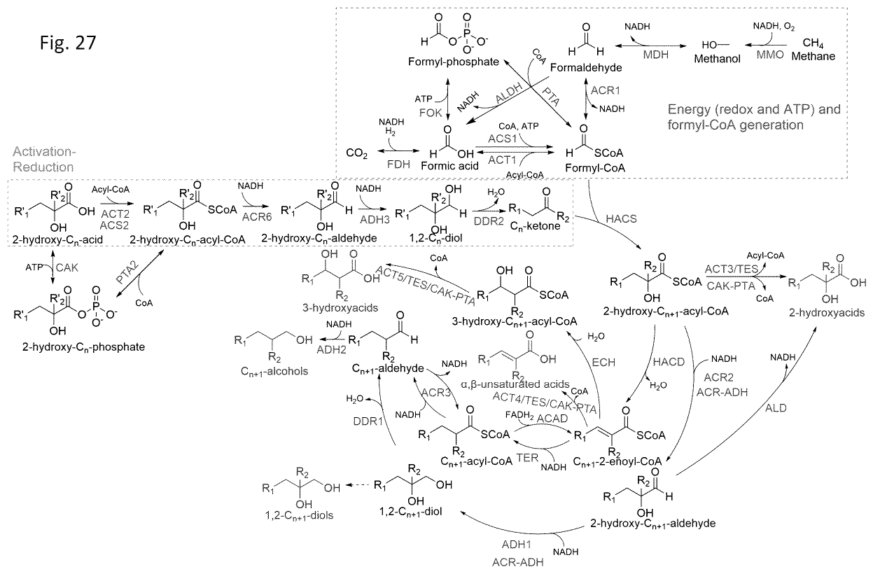

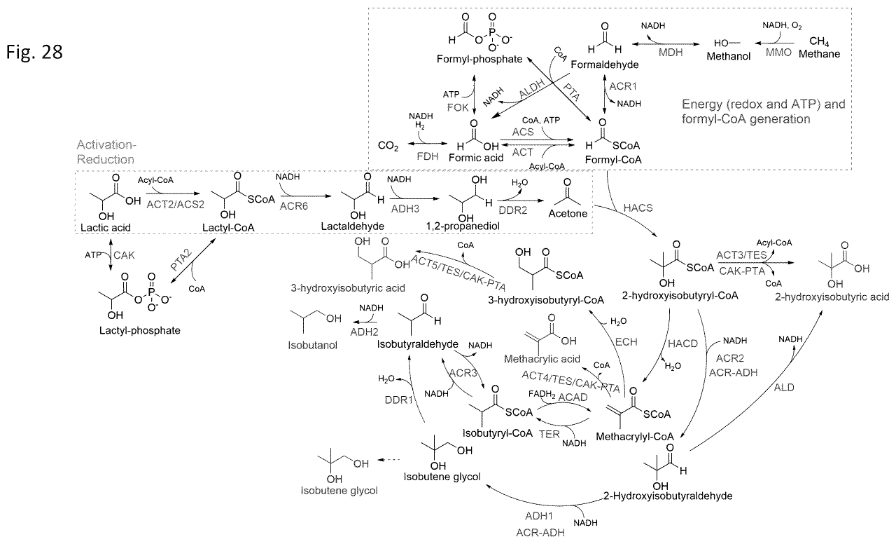

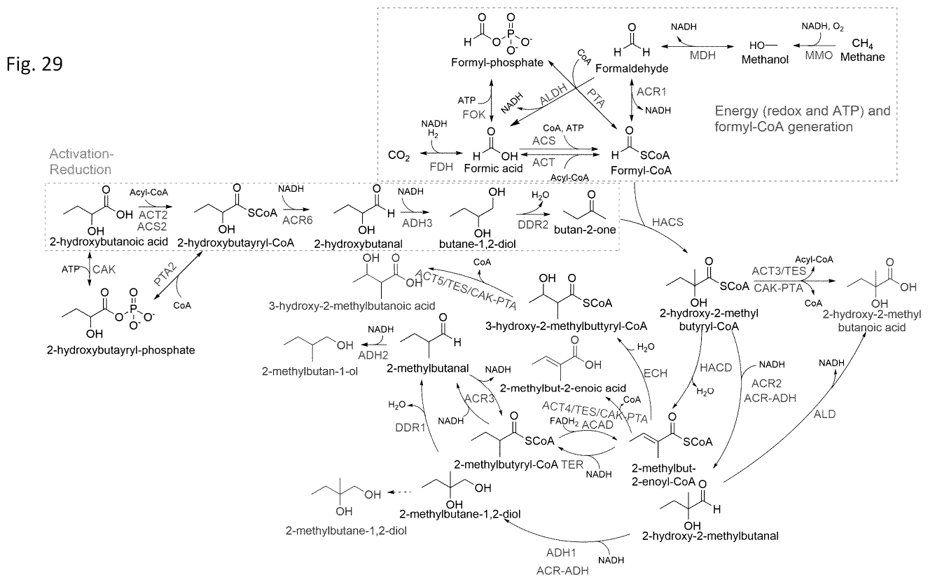

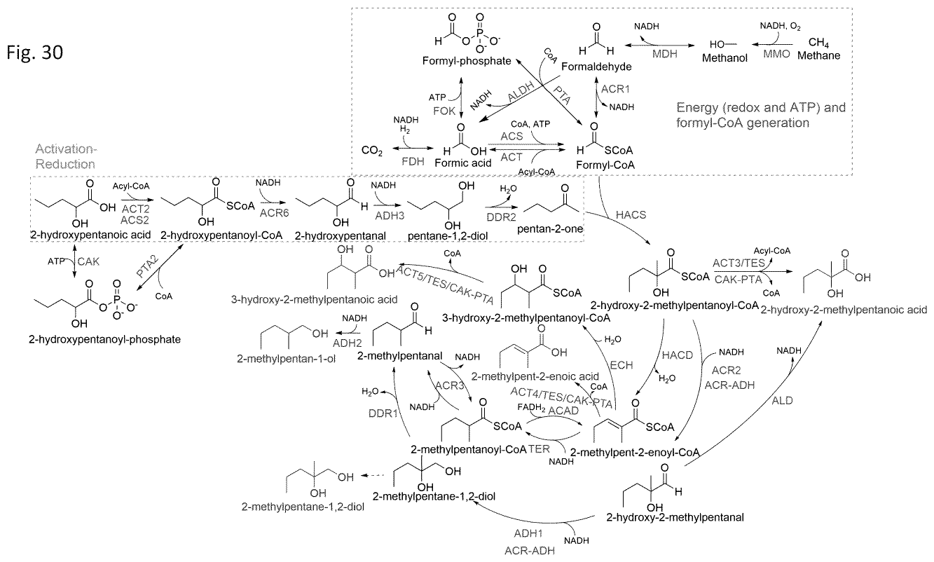

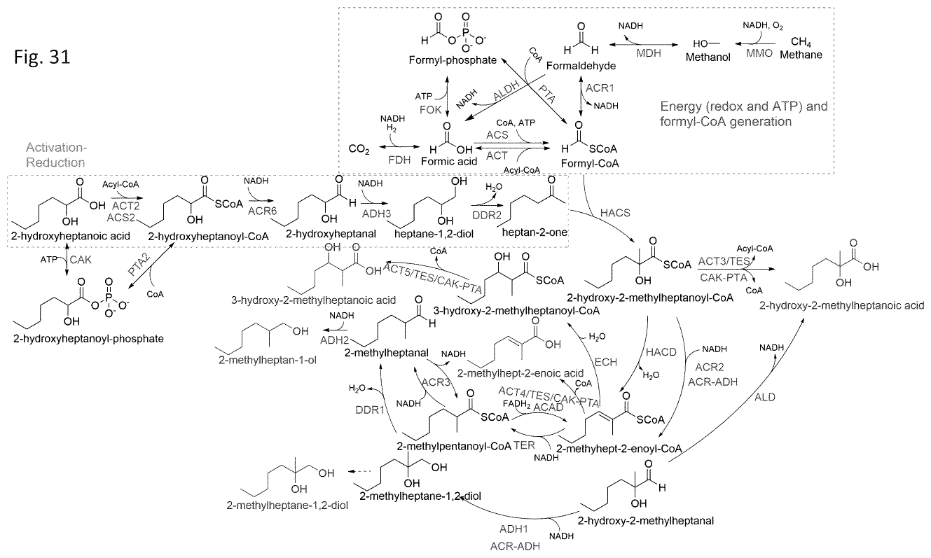

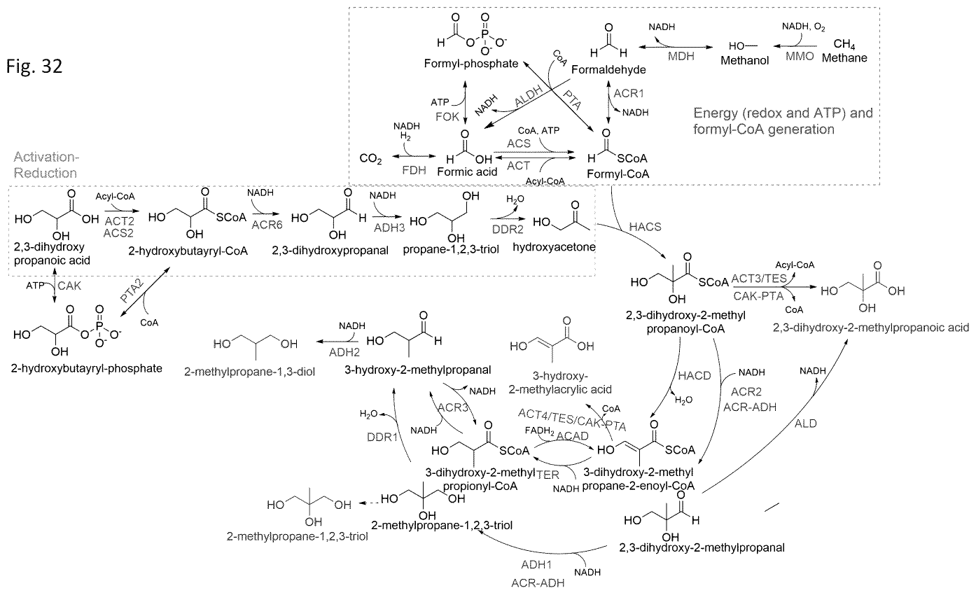

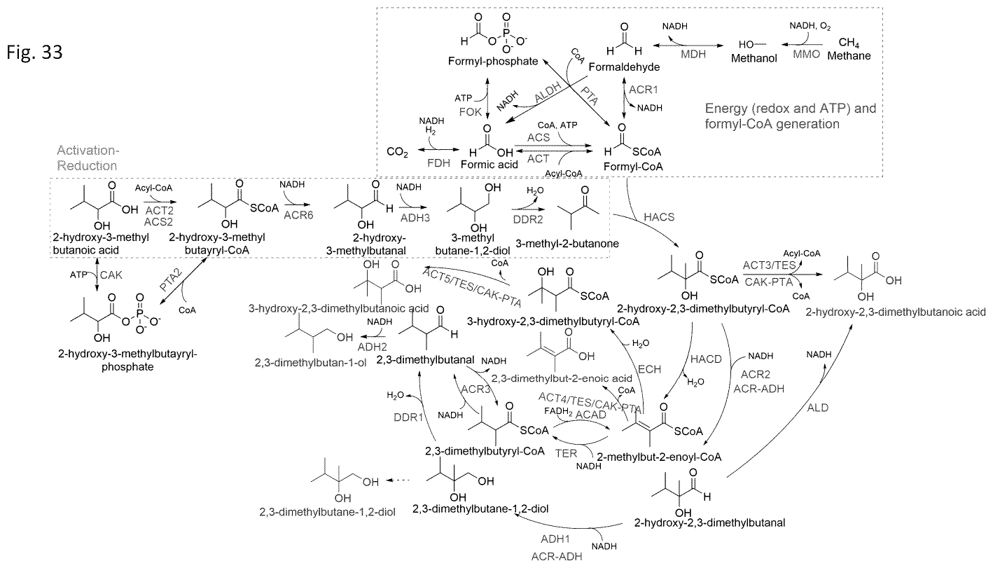

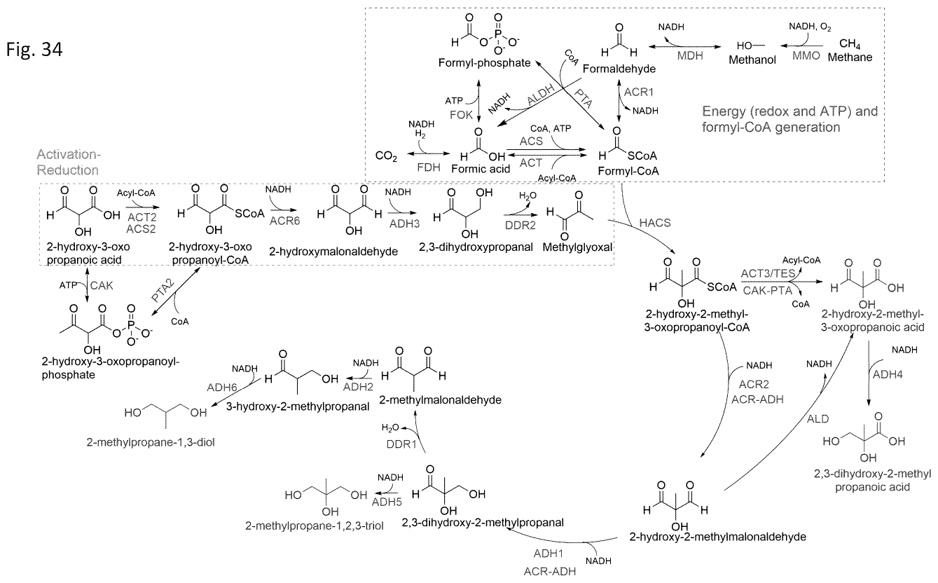

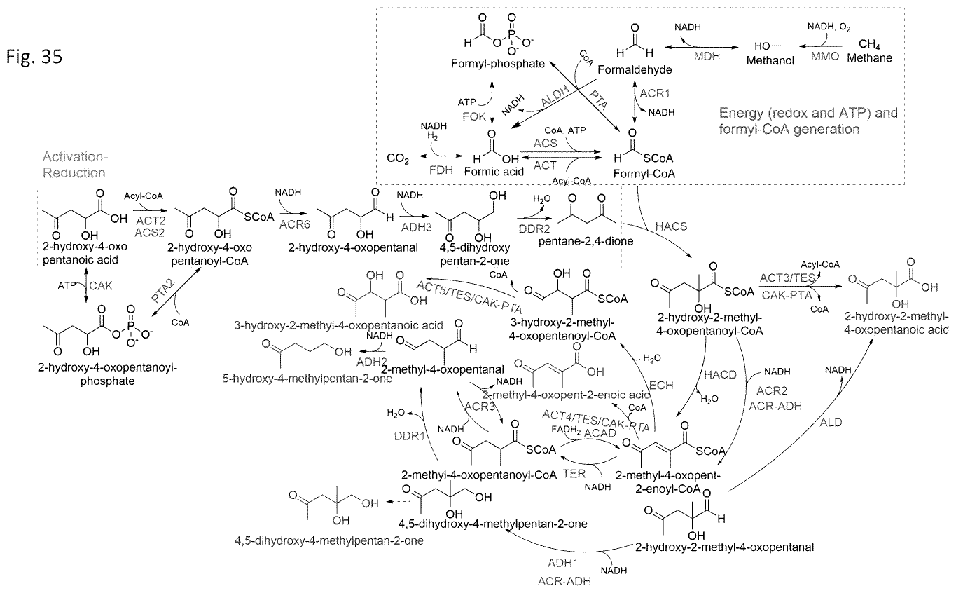

The purpose of this example is to demonstrate the implementation of the carboxylic acid (CA) platform using isovaleric acid (R═CHCH₃CH₃) as the CA intermediate with C1 elongation pathways (FIG. 26).

Isovaleric acid can be activated to isopentanoyl-CoA through isopentanoyl-phosphate. First, isovaleric acid is phosphorylated to isopentanoyl-phosphate by kinase and then converted to isopentanoyl-CoA by phosphotransacylase. The other pathway to activate isovaleric acid is CoA synthetase or CoA transferase. Finally, isopentanoyl-CoA is reduced to isovaleraldehyde by aldehyde dehydrogenase.

C1 elongation is initiated by formyl-CoA production through energy and formyl-CoA generation steps. 2-Hydroxyacyl-CoA synthase (HACS) condenses isovaleraldehyde and formyl-CoA to produce 2-hydroxy-4-methylpentanoyl-CoA. 2-Hydroxy-4-methylpentanoyl-CoA is converted to leucic acid by acyl-CoA thioesterase.

Leucic acid can be further oxidized to ketoleucine by 2-hydroxyacid dehydrogenase. Ketoleucine is reduced to leucine by amino dehydrogenase/transaminase. Dehydration of leucine produces 2-amino-4-methylpentanal which can be further reduced to leucinol or aminated to 4-methylpentane-1,2-diamine.

2-Hydroxy-4-methylpentanoyl-CoA as the product of condensation can be reduced to 4-methylpentane-1,2-diol, 4-methyl-2-pentanone and isohexanol. First, 2-hydroxy-4-methylpentanoyl-CoA is reduced to 2-hydroxy-4-methylpentanal through aldehyde dehydrogenase which can be further reduced to 4-methylpentane-1,2-diol by aldehyde reductase. Dehydration of 4-methylpentane-1,2-diol by diol dehydratase produces 4-methyl-2-pentanonc. Moreover, dehydration of 4-methylpentane-1,2-diol by diol dehydratase produces 4-methylvaleraldehyde. The resulting 4-methylvaleraldehyde can be fed to the subsequent iteration of C1 elongation or reduced to isohexanol by aldehyde reductase.

2-Hydroxy-4-methylpentanoyl-CoA can be used to produce unsaturated acids. 2-Hydroxy-4-methylpentanoyl-CoA is dehydrated to 4-methyl-2-pentenoyl-CoA by acyl-CoA dehydratase. 4-Methyl-2-pentenoyl-CoA is converted to 4-methyl-2-pentenoic acid through CoA thioesterase. Moreover, 4-methyl-2-pentenoyl-CoA can be hydrolyzed to 3-hydroxy-4-methylpentanoyl-CoA by acyl-CoA dehydratase and further oxidized to 3-hydroxy-4-methylpentanoic acid by acyl-CoA thioesterase. Another pathway for 4-methyl-2-pentenoyl-CoA is to be converted to 4-methylpentanoyl-CoA by acyl-CoA dehydrogenase. 4-Methylpentanoyl-CoA is further reduced to 4-methylvaleraldehyde by aldehyde dehydrogenase.

### Example 27: Carboxylic Acid Platform Using 2-Hydroxyacids-Derived Methyl Ketones as Substrate for Condensation with Formyl-COA

This Example demonstrates the implementation of the condensation of methyl ketones with formyl-CoA using purified enzymes. The generation of formyl-CoA catalyzed by CoA transferase and condensation catalyzed by HACS is identical to the examples described above. Methyl ketones can be produced from 2-hydroxy acids as described above or through fatty acids synthesis and β-oxidation pathway demonstrated in literatures (Appl Environ Microbiol 78:70-80, 2012; Metab Eng 62:84-94, 2020).

The enzymes acyl-CoA transferase and HACS were overexpressed and purified as described above. In vitro purified enzyme reactions for condensation of methyl ketone and formyl-CoA was comprised of 100 mM KPi pH 6.9, 10 mM MgCl₂, 0.15 mM TPP, 2 mM acetyl-CoA, 1 μM BsmHACL, 2 μM CaAbfT, 20 mM formate and 50 mM tested methyl ketones. Reactions were incubated at 30° C. for 24 hours unless otherwise specified.

For this analysis, samples containing acyl-CoAs were first treated with 1/20 of the reaction volume of 10 M NaOH solution was added to terminate the reactions. After 30 min hydrolysis, 1/20 of the reaction volume of 10 N H₂SO₄ was added to improve the efficiency of acid extraction. The resulting sample was extracted into 4 mL ethyl acetate by vigorous vortexing for 90 seconds. The organic phase was separated and evaporated to dryness under a stream of nitrogen. The residue was dissolved in 50 μL pyridine and 50 μL N,O-Bis(trimethylsilyl)trifluoroacetamide (BSTFA) and incubated at 60° C. for 15 minutes. Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 μm film thickness, 30 m length). Samples were analyzed by GC (1 μL injection with a 20:1 split ratio) using helium as the carrier gas at a flowrate of 1.5 mL/min and the following temperature profile: initial 90° C. for 3 min; ramp at 15° C./min to 170° C.; ramp at 20° C./min to 300° C. and hold for 8 min. The injector and detector temperature were 250° C. and 350° C., respectively.

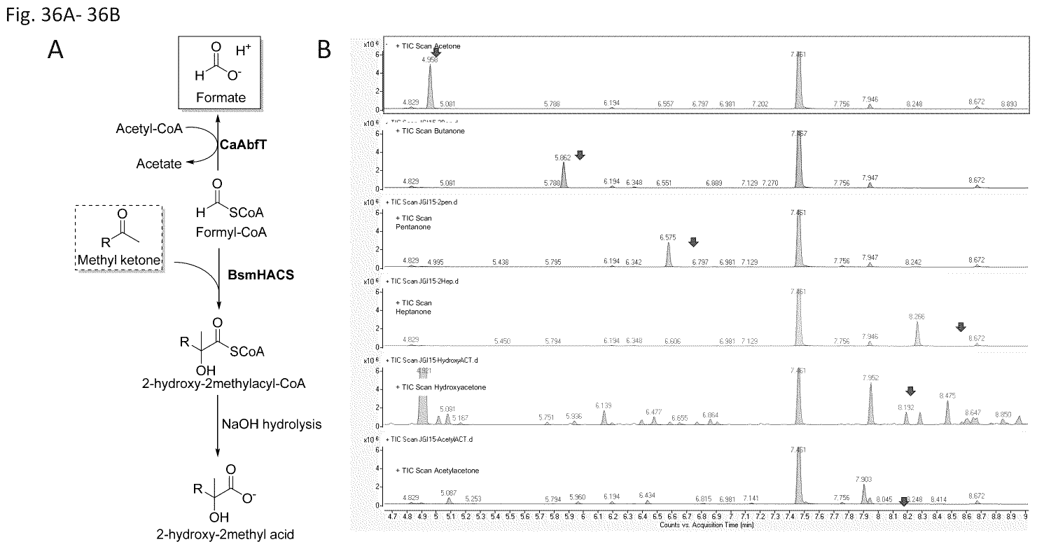

Methyl ketones that are good carbonyl-containing substrate for acyloin condensation reactions with formyl-CoA include but are not limited to acetone, methyl ethyl ketone (Cn-ketone, n>3, butanone, pentanone and heptanone as examples), hydroxylated ketones (hydroxyacetone), and other functional ketones (acetylacetone, branched-chain ketones, methylglyoxal) etc. BsmHACS was able to catalyze the condensation of all tested ketones with formyl-CoA, as shown in FIG. 36B.

### Example 28: Carboxylic Acid Platform Using Lactic Acid-Derived Acetone as Substrate for Condensation with Formyl-COA

This Example demonstrates the implementation of the condensation of methyl ketone with formyl-CoA (generated from formate) in vivo using growing cells, with acetone used as a representative methylketone. As described above, this process only needs two enzymes, the formyl-CoA generation enzyme (FAE) and the condensation enzyme (HACS). BsmHACS was cloned under control of the IPTG-inducible T7 promoter in pCDFduet-1 and FAEs under control of a cumate-inducible T5 promoter in pETDuet-1. Meanwhile, an HACL variant recently identified in Actinomycetospora chiangmaiensis DSM 45062 (Frontiers in microbiology 11:691, 2020) was also included and referred to as AcHACL

The condensation of acetone with formyl-CoA from formate in actively growing cells was conducted using the M9-LB medium containing 6.78 g/L Na₂HPO₄, 3 g/L KH₂PO₄, 1 g/L NH₄Cl, 0.5 g/L NaCl, 2 mM MgSO₄, 100 μM CaCl₂), 15 μM thiamine-HCl, 10 g/L tryptone, and 5 g/L yeast extract additionally supplemented with the micronutrient solution of Neidhardt⁶⁸. A single colony of the desired strain was cultivated overnight (14-16 hrs) in LB medium with appropriate antibiotics and used as the inoculum (1%) to 50 mL centrifugation tubes containing 5 mL of M9-LB medium. Antibiotics (100 μg/mL carbenicillin, 100 μg/mL spectinomycin) were included when appropriate. Cultures were then incubated at 30° C. and 250 rpm in a Lab Companion SI-600 rotary shaker (Jeio Tech, Seoul, South Korea) until an OD550 of ˜0.4 was reached, at which point appropriate amounts of inducer(s) (isopropyl β-D-1-thiogalactopyranoside and cumate) and substrates (acetone and formate) were added. Tubes were tightened and incubated for a total of 48 hr post-inoculation. The cells were pelleted by centrifugation and the media analyzed through HPLC or GC-MS as described above.

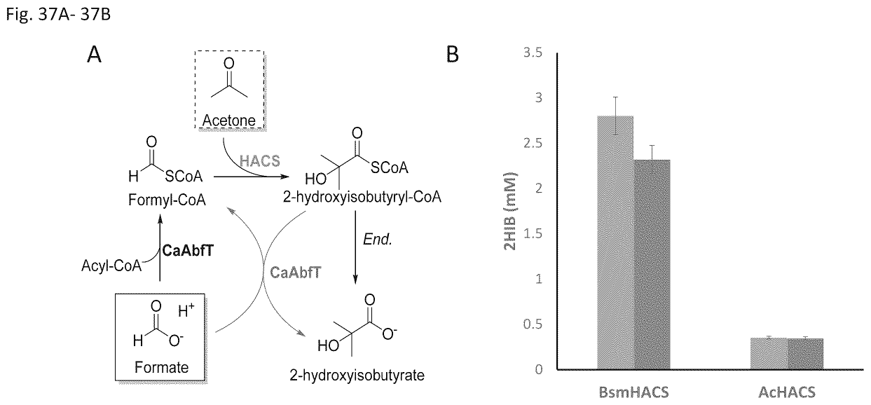

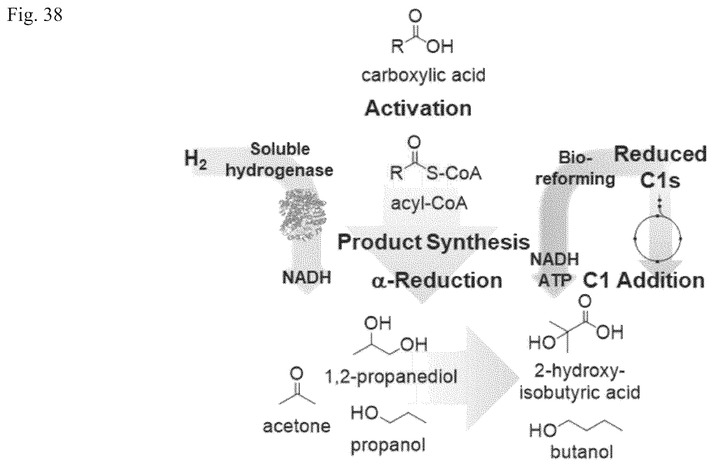

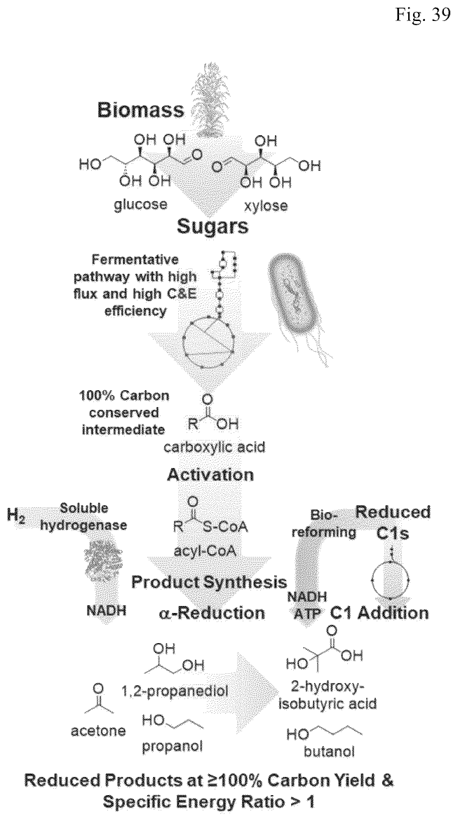

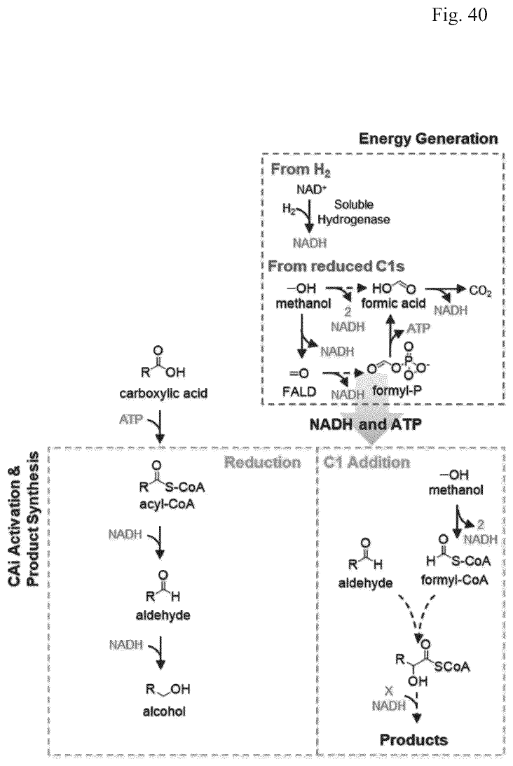

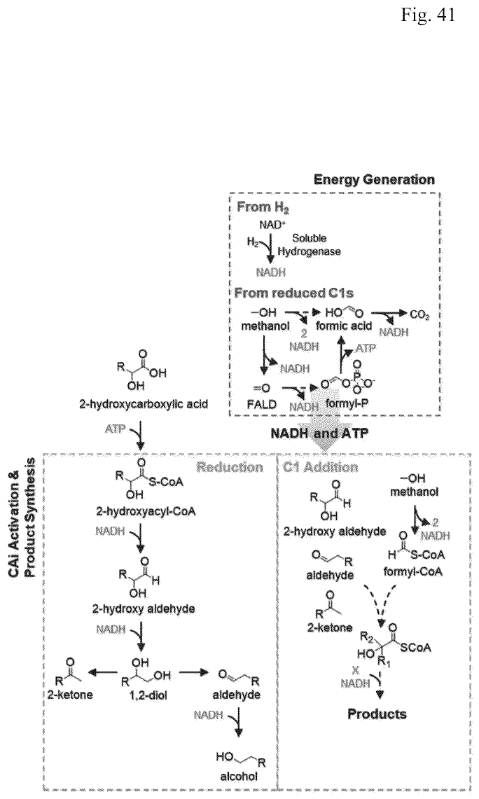

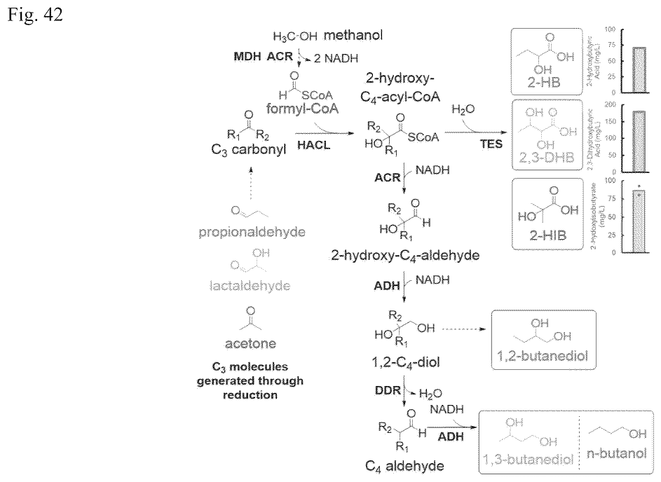

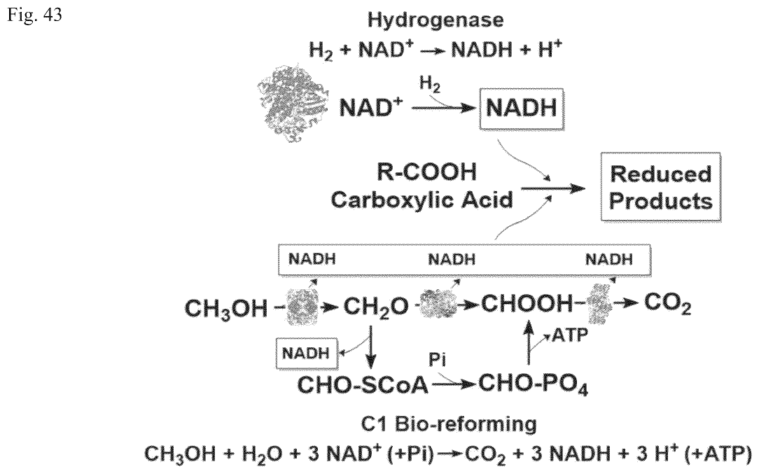

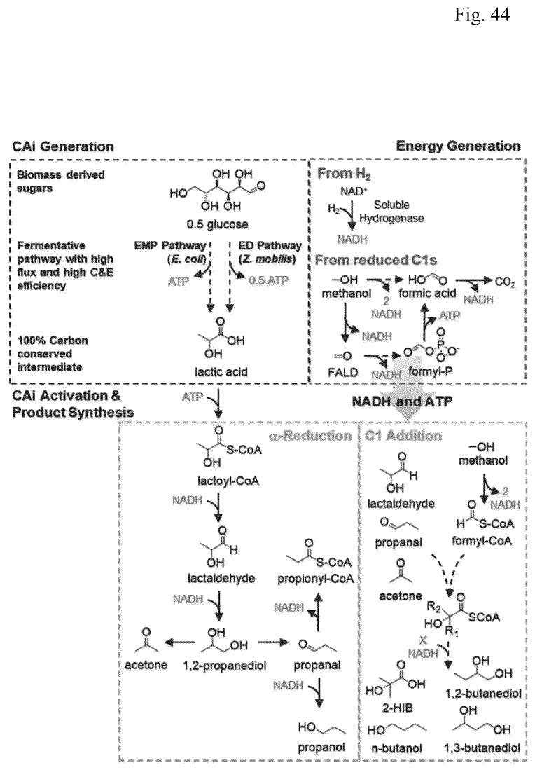

The growing cell experiment shows that both HACS catalyzed the condensation of acetone and formyl-CoA, but BsmHACS exhibited a better performance with up to 2.8 mM (291 mg/L) 2HIB produced after a 2-day fermentation (FIG. 37B).

TABLE 6
List of enzymes for carboxylic acid platform using methyl ketones for the C-1 elongation.

| | | | Uniprot | | | | Uniprot |
|---|---|---|---|---|---|---|---|
| Reaction | Gene | Organism | accession | Reaction | Gene | Organism | accession |
| sMMO | mmoXYBZCD | Methylococcus | P18797 | ADH | fucO | Escherichia | P0A9S1 |
| | | capsulatus | | | | coli | |
| pMMO | pmoA1A2B1B2 | Methylococcus | G1UBD1 | | gldA | Escherichia | P0A9S5 |
| | | capsulatus | Q607G3 | | | coli | |
| MDH | BmMDH2 | Bacillus | I3E2P9 | | rhaZ | Salmonella | Q8ZKS2 |
| | | methanolicus MGA3 | | | | typhimurium | |
| | CnMDH2 | Cupriavidus necator | F8GNE5 | | yahK | Escherichia | P75691 |
| | | | | | | coli | |
| | BsMDH | Bacillus | P42327 | | adhA | Lactococcus | Q9CEN0 |
| | | stearothermophilus | | | | lactis | |
| ACR | LmACR | Listeria | Q8Y7U1 | | yjgB | Escherichia | P27250 |
| | | monocytogenes | | | | coli | |
| | StEutE | Salmonella | P41793 | | yqhD | Escherichia | Q46856 |
| | | typhymurium | | | | coli | |
| | CbAld | Clostridium | Q716S8 | | pduQ | Salmonella | Q9XDN0 |
| | | beijerinckii | | | | enterica | |
| | EcMhpF | Escherichia | P77580 | | YLL056C | Saccharomyces | Q12177 |
| | | coli | | | | cerevisiae | |
| | PsDmpF | Pseudomonas sp. | Q52060 | DDR | RiDD | Roseburia | Q1A666 |
| | | strain CF600 | | | | inulinivorans | |
| | pduP | Salmonella | Q9XDN1 | | pduCDE | Salmonella | P37450 |
| | | enterica | | | | enterica | |
| | AtAdhE | Acetivibrio | A3DCI2 | | pddABC | Klebsiella | Q59470 |
| | | thermocellus | | | | oxytoca | |
| | EcMhpF | Escherichia | P77580 | HACD | BpCaiD_2 | Burkholderia | A0A1X1PK59 |
| | | coli | | | | puraquae | |
| PTA-FOK | CcPta-Ack | Clostridium | A0A0J8D6J2 | | CdHadBC | Clostridioides | Q5U923 |
| | | cylindrosporum | A0A0J8DB00 | | | difficile | |
| | EcPta-Ack | Escherichia | P0A9M8 | ACAD | EcPaaZ | Escherichia | P77455 |
| | | coli | P0A6A3 | | | coli | |
| ACS | EcACS | Escherichia | P27550 | | AtACX4 | Arabidopsis | Q96329 |
| | | coli | | | | thaliana | |
| | StACSstab | Salmonella | Q8ZKF6_PROSS | TER | EgTER | Euglena | Q5EU90 |
| | | typhimurium | | | | gracilis | |
| | MhACS | Marinithermus | F2NQX2 | ECH | CaCRT | Clostridium | P52046 |
| | | hydrothemalis | | | | acetobutylicum | |
| | ArACS | Angustibacter sp. | A0A0Q7JEV7 | | PfECH | Pseudomonas | C3K613 |
| | | Root456 | | | | fluorescens | |
| HACS | RuHACS | Rhodospirillales | A0A1H8YFL8 | ALD | CaHbd | Clostridium | P52041 |
| | | bacterium URHD0017 | | | | acetobutylicum | |
| | BsmHACS | beach sand | A0A3C0TX30 | | EcAldA | Escherichia | P25553 |
| | | metagenome | | | | coli | |
| | DbHACS | Dehalococcoidia | A0A315XEK8 | ACT | CaAbfT | Clostridium | Q9RM86 |
| | | bacterium | | | | aminobutyricum | |
| | AcHACS | Actinomycetospora | P0DUV9 | | OfFrc | Oxalobacter | O06644 |
| | | chiangmaiensis | | | | formigenes | |
| ALDH | EcFrmA | Escherichia | P25437 | FDH | PsFdh | Pseudomonas sp. | P33160 |
| | | coli | | | | (strain 101) | |
| | PpFdhA | Pseudomonas | P46154 | | CbFdh | Candida | O13437 |
| | | putida | | | | boidinii | |
| | | | | TES | EcYciA | Escherichia | P0A8Z0 |
| | | | | | | coli | |

TABLE 7
List of enzymes for carboxylic acid platform using acetone for the C-1 elongation.

| | | | Uniprot | | | | Uniprot |
|---|---|---|---|---|---|---|---|
| Reaction | Gene | Organism | accession | Reaction | Gene | Organism | accession |
| sMMO | mmoXYBZCD | Methylococcus | P18797 | ADH | fucO | Escherichia | P0A9S1 |
| | | capsulatus | | | | coli | |
| pMMO | pmoA1A2B1B2 | Methylococcus | G1UBD1 | | gldA | Escherichia | P0A9S5 |
| | | capsulatus | Q607G3 | | | coli | |
| MDH | BmMDH2 | Bacillus | I3E2P9 | | rhaZ | Salmonella | Q8ZKS2 |
| | | methanolicus MGA3 | | | | typhimurium | |
| | CnMDH2 | Cupriavidus | F8GNE5 | | yahK | Escherichia | P75691 |
| | | necator | | | | coli | |
| | BsMDH | Bacillus | P42327 | | adhA | Lactococcus | Q9CEN0 |
| | | stearothermophilus | | | | lactis | |
| ACR | LmACR | Listeria | Q8Y7U1 | | yjgB | Escherichia | P27250 |
| | | monocytogenes | | | | coli | |
| | StEutE | Salmonella | P41793 | | yqhD | Escherichia | Q46856 |
| | | typhymurium | | | | coli | |
| | CbAld | Clostridium | Q716S8 | | pduQ | Salmonella | Q9XDN0 |
| | | beijerinckii | | | | enterica | |
| | EcMhpF | Escherichia | P77580 | | YLL056C | Saccharomyces | Q12177 |
| | | coli | | | | cerevisiae | |
| | PsDmpF | Pseudomonas sp. | Q52060 | DDR | RiDD | Roseburia | Q1A666 |
| | | strain CF600 | | | | inulinivorans | |
| | pduP | Salmonella | Q9XDN1 | | pduCDE | Salmonella | P37450 |
| | | enterica | | | | enterica | |
| | AtAdhE | Acetivibrio | A3DCI2 | | pddABC | Klebsiella | Q59470 |
| | | thermocellus | | | | oxytoca | |
| | EcMhpF | Escherichia | P77580 | HACD | BpCaiD_2 | Burkholderia | A0A1X1PK59 |
| | | coli | | | | puraquae | |
| PTA-FOK | CcPta-Ack | Clostridium | A0A0J8D6J2 | | CdHadBC | Clostridioides | Q5U923 |
| | | cylindrosporum | A0A0J8DB00 | | | difficile | |
| | EcPta-Ack | Escherichia | P0A9M8 | ACAD | EcPaaZ | Escherichia | P77455 |
| | | coli | P0A6A3 | | | coli | |
| ACS | EcACS | Escherichia | P27550 | | AtACX4 | Arabidopsis | Q96329 |
| | | coli | | | | thaliana | |
| | StACSstab | Salmonella | Q8ZKF6_PROSS | TER | EgTER | Euglena | QSEU90 |
| | | typhimurium | | | | gracilis | |
| | MhACS | Marinithermus | F2NQX2 | ECH | CaCRT | Clostridium | P52046 |
| | | hydrothemalis | | | | acetobutylicum | |
| | ArACS | Angustibacter sp. | A0A0Q7JEV7 | | PfECH | Pseudomonas | C3K613 |
| | | Root456 | | | | fluorescens | |
| HACS | RuHACS | Rhodospirillales | A0A1H8YFL8 | ALD | CaHbd | Clostridium | P52041 |
| | | bacterium URHD0017 | | | | acetobutylicum | |
| | BsmHACS | beach sand | A0A3C0TX30 | | EcAldA | Escherichia | P25553 |
| | | metagenome | | | | coli | |
| | DbHACS | Dehalococcoidia | A0A315XEK8 | ACT | CaAbfT | Clostridium | Q9RM86 |
| | | bacterium | | | | aminobutyricum | |
| | AcHACS | Actinomycetospora | P0DUV9 | | OfFrc | Oxalobacter | O06644 |
| | | chiangmaiensis | | | | formigenes | |
| ALDH | EcFrmA | Escherichia | P25437 | FDH | PsFdh | Pseudomonas sp. | P33160 |
| | | coli | | | | (strain 101) | |
| | PpFdhA | Pseudomonas | P46154 | | CbFdh | Candida | O13437 |
| | | putida | | | | boidinii | |
| | | | | TES | EcYciA | Escherichia | P0A8Z0 |
| | | | | | | coli | |

TABLE 8
List of enzymes for carboxylic acid platform using butanone for the C-1 elongation

| | | | Uniprot | | | | Uniprot |
|---|---|---|---|---|---|---|---|
| Reaction | Gene | Organism | accession | Reaction | Gene | Organism | accession |
| sMMO | mmoXYBZCD | Methylococcus | P18797 | ADH | fucO | Escherichia | P0A9S1 |
| | | capsulatus | | | | coli | |
| pMMO | pmoA1A2B1B2 | Methylococcus | G1UBD1 | | gldA | Escherichia | P0A9S5 |
| | | capsulatus | Q607G3 | | | coli | |
| MDH | BmMDH2 | Bacillus | I3E2P9 | | rhaZ | Salmonella | Q8ZKS2 |
| | | methanolicus MGA3 | | | | typhimurium | |
| | CnMDH2 | Cupriavidus | F8GNE5 | | yahK | Escherichia | P75691 |
| | | necator | | | | coli | |
| | BsMDH | Bacillus | P42327 | | adhA | Lactococcus | Q9CEN0 |
| | | stearothermophilus | | | | lactis | |
| ACR | LmACR | Listeria | Q8Y7U1 | | yjgB | Escherichia | P27250 |
| | | monocytogenes | | | | coli | |
| | StEutE | Salmonella | P41793 | | yqhD | Escherichia | Q46856 |
| | | typhymurium | | | | coli | |
| | CbAld | Clostridium | Q716S8 | | pduQ | Salmonella | Q9XDN0 |
| | | beijerinckii | | | | enterica | |
| | EcMhpF | Escherichia | P77580 | | YLL056C | Saccharomyces | Q12177 |
| | | coli | | | | cerevisiae | |
| | PsDmpF | Pseudomonas sp. | Q52060 | DDR | RiDD | Roseburia | Q1A666 |
| | | strain CF600 | | | | inulinivorans | |
| | pduP | Salmonella | Q9XDN1 | | pduCDE | Salmonella | P37450 |
| | | enterica | | | | enterica | |
| | AtAdhE | Acetivibrio | A3DCI2 | | pddABC | Klebsiella | Q59470 |
| | | thermocellus | | | | oxytoca | |
| | EcMhpF | Escherichia | P77580 | HACD | BpCaiD_2 | Burkholderia | A0A1X1PK59 |
| | | coli | | | | puraquae | |
| PTA-FOK | CcPta-Ack | Clostridium | A0A0J8D6J2 | | CdHadBC | Clostridioides | Q5U923 |
| | | cylindrosporum | A0A0J8DB00 | | | difficile | |
| | EcPta-Ack | Escherichia | P0A9M8 | ACAD | EcPaaZ | Escherichia | P77455 |
| | | coli | P0A6A3 | | | coli | |
| ACS | EcACS | Escherichia | P27550 | | AtACX4 | Arabidopsis | Q96329 |
| | | coli | | | | thaliana | |
| | StACSstab | Salmonella | Q8ZKF6_PROSS | TER | EgTER | Euglena | Q5EU90 |
| | | typhimurium | | | | gracilis | |
| | MhACS | Marinithermus | F2NQX2 | ECH | CaCRT | Clostridium | P52046 |
| | | hydrothemalis | | | | acetobutylicum | |
| | ArACS | Angustibacter sp. | A0A0Q7JEV7 | | PfECH | Pseudomonas | C3K613 |
| | | Root456 | | | | fluorescens | |
| HACS | RuHACS | Rhodospirillales | A0A1H8YFL8 | ALD | CaHbd | Clostridium | P52041 |
| | | bacterium URHD0017 | | | | acetobutylicum | |
| | BsmHACS | beach sand | A0A3C0TX30 | | EcAldA | Escherichia | P25553 |
| | | metagenome | | | | coli | |
| | DbHACS | Dehalococcoidia | A0A315XEK8 | ACT | CaAbfT | Clostridium | Q9RM86 |
| | | bacterium | | | | aminobutyricum | |
| | AcHACS | Actinomycetospora | P0DUV9 | | OfFrc | Oxalobacter | O06644 |
| | | chiangmaiensis | | | | formigenes | |
| ALDH | EcFrmA | Escherichia | P25437 | FDH | PsFdh | Pseudomonas sp. | P33160 |
| | | coli | | | | (strain 101) | |
| | PpFdhA | Pseudomonas | P46154 | | CbFdh | Candida | O13437 |
| | | putida | | | | boidinii | |
| | | | | TES | EcYciA | Escherichia | P0A8Z0 |
| | | | | | | coli | |

TABLE 9
List of enzymes for carboxylic acid platform using pentanone for the C-1 elongation

| | | | Uniprot | | | | Uniprot |
|---|---|---|---|---|---|---|---|
| Reaction | Gene | Organism | accession | Reaction | Gene | Organism | accession |
| sMMO | mmoXYBZCD | Methylococcus | P18797 | ADH | fucO | Escherichia | P0A9S1 |
| | | capsulatus | | | | coli | |
| pMMO | pmoA1A2B1B2 | Methylococcus | G1UBD1 | | gldA | Escherichia | P0A9S5 |
| | | capsulatus | Q607G3 | | | coli | |
| MDH | BmMDH2 | Bacillus | I3E2P9 | | rhaZ | Salmonella | Q8ZKS2 |
| | | methanolicus MGA3 | | | | typhimurium | |
| | CnMDH2 | Cupriavidus | F8GNE5 | | yahK | Escherichia | P75691 |
| | | necator | | | | coli | |
| | BsMDH | Bacillus | P42327 | | adhA | Lactococcus | Q9CEN0 |
| | | stearothermophilus | | | | lactis | |
| ACR | LmACR | Listeria | Q8Y7U1 | | yjgB | Escherichia | P27250 |
| | | monocytogenes | | | | coli | |
| | StEutE | Salmonella | P41793 | | yqhD | Escherichia | Q46856 |
| | | typhymurium | | | | coli | |
| | CbAld | Clostridium | Q716S8 | | pduQ | Salmonella | Q9XDN0 |
| | | beijerinckii | | | | enterica | |
| | EcMhpF | Escherichia | P77580 | | YLL056C | Saccharomyces | Q12177 |
| | | coli | | | | cerevisiae | |
| | PsDmpF | Pseudomonas sp. | Q52060 | DDR | RiDD | Roseburia | Q1A666 |
| | | strain CF600 | | | | inulinivorans | |
| | pduP | Salmonella | Q9XDN1 | | pduCDE | Salmonella | P37450 |
| | | enterica | | | | enterica | |
| | AtAdhE | Acetivibrio | A3DCI2 | | pddABC | Klebsiella | Q59470 |
| | | thermocellus | | | | oxytoca | |
| | EcMhpF | Escherichia | P77580 | HACD | BpCaiD_2 | Burkholderia | A0A1X1PK59 |
| | | coli | | | | puraquae | |
| PTA-FOK | CcPta-Ack | Clostridium | A0A0J8D6J2 | | CdHadBC | Clostridioides | Q5U923 |
| | | cylindrosporum | A0A0J8DB00 | | | difficile | |
| | EcPta-Ack | Escherichia | P0A9M8 | ACAD | EcPaaZ | Escherichia | P77455 |
| | | coli | P0A6A3 | | | coli | |
| ACS | EcACS | Escherichia | P27550 | | AtACX4 | Arabidopsis | Q96329 |
| | | coli | | | | thaliana | |
| | StACSstab | Salmonella | Q8ZKF6_PROSS | TER | EgTER | Euglena | Q5EU90 |
| | | typhimurium | | | | gracilis | |
| | MhACS | Marinithermus | F2NQX2 | ECH | CaCRT | Clostridium | P52046 |
| | | hydrothemalis | | | | acetobutylicum | |
| | ArACS | Angustibacter sp. | A0A0Q7JEV7 | | PfECH | Pseudomonas | C3K613 |
| | | Root456 | | | | fluorescens | |
| HACS | RuHACS | Rhodospirillales | A0A1H8YFL8 | ALD | CaHbd | Clostridium | P52041 |
| | | bacterium URHD0017 | | | | acetobutylicum | |
| | BsmHACS | beach sand | A0A3C0TX30 | | EcAldA | Escherichia | P25553 |
| | | metagenome | | | | coli | |
| | DbHACS | Dehalococcoidia | A0A315XEK8 | ACT | CaAbfT | Clostridium | Q9RM86 |
| | | bacterium | | | | aminobutyricum | |
| | AcHACS | Actinomycetospora | P0DUV9 | | OfFrc | Oxalobacter | O06644 |
| | | chiangmaiensis | | | | formigenes | |
| ALDH | EcFrmA | Escherichia | P25437 | FDH | PsFdh | Pseudomonas sp. | P33160 |
| | | coli | | | | (strain 101) | |
| | PpFdhA | Pseudomonas | P46154 | | CbFdh | Candida | O13437 |
| | | putida | | | | boidinii | |
| | | | | TES | EcYciA | Escherichia | P0A8Z0 |
| | | | | | | coli | |

TABLE 10
List of enzymes for carboxylic acid platform using heptanone for the C-1 elongation

| | | | Uniprot | | | | Uniprot |
|---|---|---|---|---|---|---|---|
| Reaction | Gene | Organism | accession | Reaction | Gene | Organism | accession |
| sMMO | mmoXYBZCD | Methylococcus | P18797 | ADH | fucO | Escherichia | P0A9S1 |
| | | capsulatus | | | | coli | |
| pMMO | pmoA1A2B1B2 | Methylococcus | G1UBD1 | | gldA | Escherichia | P0A9S5 |
| | | capsulatus | Q607G3 | | | coli | |
| MDH | BmMDH2 | Bacillus | I3E2P9 | | rhaZ | Salmonella | Q8ZKS2 |
| | | methanolicus MGA3 | | | | typhimurium | |
| | CnMDH2 | Cupriavidus | F8GNE5 | | yahK | Escherichia | P75691 |
| | | necator | | | | coli | |
| | BsMDH | Bacillus | P42327 | | adhA | Lactococcus | Q9CEN0 |
| | | stearothermophilus | | | | lactis | |
| ACR | LmACR | Listeria | Q8Y7U1 | | yjgB | Escherichia | P27250 |
| | | monocytogenes | | | | coli | |
| | StEutE | Salmonella | P41793 | | yqhD | Escherichia | Q46856 |
| | | typhymurium | | | | coli | |
| | CbAld | Clostridium | Q716S8 | | pduQ | Salmonella | Q9XDN0 |
| | | beijerinckii | | | | enterica | |
| | EcMhpF | Escherichia | P77580 | | YLL056C | Saccharomyces | Q12177 |
| | | coli | | | | cerevisiae | |
| | PsDmpF | Pseudomonas sp. | Q52060 | DDR | RiDD | Roseburia | Q1A666 |
| | | strain CF600 | | | | inulinivorans | |
| | pduP | Salmonella | Q9XDN1 | | pduCDE | Salmonella | P37450 |
| | | enterica | | | | enterica | |
| | AtAdhE | Acetivibrio | A3DCI2 | | pddABC | Klebsiella | Q59470 |
| | | thermocellus | | | | oxytoca | |
| | EcMhpF | Escherichia | P77580 | HACD | BpCaiD_2 | Burkholderia | A0A1X1PK59 |
| | | coli | | | | puraquae | |
| PTA-FOK | CcPta-Ack | Clostridium | A0A0J8D6J2 | | CdHadBC | Clostridioides | Q5U923 |
| | | cylindrosporum | A0A0J8DB00 | | | difficile | |
| | EcPta-Ack | Escherichia | P0A9M8 | ACAD | EcPaaZ | Escherichia | P77455 |
| | | coli | P0A6A3 | | | coli | |
| ACS | EcACS | Escherichia | P27550 | | AtACX4 | Arabidopsis | Q96329 |
| | | coli | | | | thaliana | |
| | StACSstab | Salmonella | Q8ZKF6_PROSS | TER | EgTER | Euglena | Q5EU90 |
| | | typhimurium | | | | gracilis | |
| | MhACS | Marinithermus | F2NQX2 | ECH | CaCRT | Clostridium | P52046 |
| | | hydrothemalis | | | | acetobutylicum | |
| | ArACS | Angustibacter sp. | A0A0Q7JEV7 | | PfECH | Pseudomonas | C3K613 |
| | | Root456 | | | | fluorescens | |
| HACS | RuHACS | Rhodospirillales | A0A1H8YFL8 | ALD | CaHbd | Clostridium | P52041 |
| | | bacterium URHD0017 | | | | acetobutylicum | |
| | BsmHACS | beach sand | A0A3C0TX30 | | EcAldA | Escherichia | P25553 |
| | | metagenome | | | | coli | |
| | DbHACS | Dehalococcoidia | A0A315XEK8 | ACT | CaAbfT | Clostridium | Q9RM86 |
| | | bacterium | | | | aminobutyricum | |
| | AcHACS | Actinomycetospora | P0DUV9 | | OfFrc | Oxalobacter | O06644 |
| | | chiangmaiensis | | | | formigenes | |
| ALDH | EcFrmA | Escherichia | P25437 | FDH | PsFdh | Pseudomonas sp. | P33160 |
| | | coli | | | | (strain 101) | |
| | PpFdhA | Pseudomonas | P46154 | | CbFdh | Candida | O13437 |
| | | putida | | | | boidinii | |
| | | | | TES | EcYciA | Escherichia | P0A8Z0 |
| | | | | | | coli | |

TABLE 11
List of enzymes for carboxylic acid platform using hydroxyacetone for the C-1 elongation

| | | | Uniprot | | | | Uniprot |
|---|---|---|---|---|---|---|---|
| Reaction | Gene | Organism | accession | Reaction | Gene | Organism | accession |
| sMMO | mmoXYBZCD | Methylococcus | P18797 | ADH | fucO | Escherichia | P0A9S1 |
| | | capsulatus | | | | coli | |
| pMMO | pmoA1A2B1B2 | Methylococcus | G1UBD1 | | gldA | Escherichia | P0A9S5 |
| | | capsulatus | Q607G3 | | | coli | |
| MDH | BmMDH2 | Bacillus | I3E2P9 | | rhaZ | Salmonella | Q8ZKS2 |
| | | methanolicus MGA3 | | | | typhimurium | |
| | CnMDH2 | Cupriavidus | F8GNE5 | | yahK | Escherichia | P75691 |
| | | necator | | | | coli | |
| | BsMDH | Bacillus | P42327 | | adhA | Lactococcus | Q9CEN0 |
| | | stearothermophilus | | | | lactis | |
| ACR | LmACR | Listeria | Q8Y7U1 | | yjgB | Escherichia | P27250 |
| | | monocytogenes | | | | coli | |
| | StEutE | Salmonella | P41793 | | yqhD | Escherichia | Q46856 |
| | | typhymurium | | | | coli | |
| | CbAld | Clostridium | Q716S8 | | pduQ | Salmonella | Q9XDN0 |
| | | beijerinckii | | | | enterica | |
| | EcMhpF | Escherichia | P77580 | | YLL056C | Saccharomyces | Q12177 |
| | | coli | | | | cerevisiae | |
| | PsDmpF | Pseudomonas sp. | Q52060 | DDR | RiDD | Roseburia | Q1A666 |
| | | strain CF600 | | | | inulinivorans | |
| | pduP | Salmonella | Q9XDN1 | | pduCDE | Salmonella | P37450 |
| | | enterica | | | | enterica | |
| | AtAdhE | Acetivibrio | A3DCI2 | | pddABC | Klebsiella | Q59470 |
| | | thermocellus | | | | oxytoca | |
| | EcMhpF | Escherichia | P77580 | HACD | BpCaiD_2 | Burkholderia | A0A1X1PK59 |
| | | coli | | | | puraquae | |
| PTA-FOK | CcPta-Ack | Clostridium | A0A0J8D6J2 | | CdHadBC | Clostridioides | Q5U923 |
| | | cylindrosporum | A0A0J8DB00 | | | difficile | |
| | EcPta-Ack | Escherichia | P0A9M8 | ACAD | EcPaaZ | Escherichia | P77455 |
| | | coli | P0A6A3 | | | coli | |
| ACS | EcACS | Escherichia | P27550 | | AtACX4 | Arabidopsis | Q96329 |
| | | coli | | | | thaliana | |
| | StACSstab | Salmonella | Q8ZKF6_PROSS | TER | EgTER | Euglena | Q5EU90 |
| | | typhimurium | | | | gracilis | |
| | MhACS | Marinithermus | F2NQX2 | ECH | CaCRT | Clostridium | P52046 |
| | | hydrothemalis | | | | acetobutylicum | |
| | ArACS | Angustibacter sp. | A0A0Q7JEV7 | | PfECH | Pseudomonas | C3K613 |
| | | Root456 | | | | fluorescens | |
| HACS | RuHACS | Rhodospirillales | A0A1H8YFL8 | ALD | CaHbd | Clostridium | P52041 |
| | | bacterium URHD0017 | | | | acetobutylicum | |
| | BsmHACS | beach sand | A0A3C0TX30 | | EcAldA | Escherichia | P25553 |
| | | metagenome | | | | coli | |
| | DbHACS | Dehalococcoidia | A0A315XEK8 | ACT | CaAbfT | Clostridium | Q9RM86 |
| | | bacterium | | | | aminobutyricum | |
| | AcHACS | Actinomycetospora | P0DUV9 | | OfFrc | Oxalobacter | O06644 |
| | | chiangmaiensis | | | | formigenes | |
| ALDH | EcFrmA | Escherichia | P25437 | FDH | PsFdh | Pseudomonas sp. | P33160 |
| | | coli | | | | (strain 101) | |
| | PpFdhA | Pseudomonas | P46154 | | CbFdh | Candida | O13437 |
| | | putida | | | | boidinii | |
| | | | | TES | EcYciA | Escherichia | P0A8Z0 |
| | | | | | | coli | |

TABLE 12
List of enzymes for carboxylic acid platform using 3-methyl-2-butanone for the C-1 elongation

| | | | Uniprot | | | | Uniprot |
|---|---|---|---|---|---|---|---|
| Reaction | Gene | Organism | accession | Reaction | Gene | Organism | accession |
| sMMO | mmoXYBZCD | Methylococcus | P18797 | ADH | fucO | Escherichia | P0A9S1 |
| | | capsulatus | | | | coli | |
| pMMO | pmoA1A2B1B2 | Methylococcus | G1UBD1 | | gldA | Escherichia coli | P0A9S5 |
| | | capsulatus | Q607G3 | | | | |
| MDH | BmMDH2 | Bacillus | I3E2P9 | | rhaZ | Salmonella | Q8ZKS2 |
| | | methanolicus MGA3 | | | | typhimurium | |
| | CnMDH2 | Cupriavidus | F8GNE5 | | yahK | Escherichia | P75691 |
| | | necator | | | | coli | |
| | BsMDH | Bacillus | P42327 | | adhA | Lactococcus | Q9CEN0 |
| | | stearothermophilus | | | | lactis | |
| ACR | LmACR | Listeria | Q8Y7U1 | | yjgB | Escherichia | P27250 |
| | | monocytogenes | | | | coli | |
| | StEutE | Salmonella | P41793 | | yqhD | Escherichia | Q46856 |
| | | typhymurium | | | | coli | |
| | CbAld | Clostridium | Q716S8 | | pduQ | Salmonella | Q9XDN0 |
| | | beijerinckii | | | | enterica | |
| | EcMhpF | Escherichia | P77580 | | YLL056C | Saccharomyces | Q12177 |
| | | coli | | | | cerevisiae | |
| | PsDmpF | Pseudomonas sp. | Q52060 | DDR | RiDD | Roseburia | Q1A666 |
| | | strain CF600 | | | | inulinivorans | |
| | pduP | Salmonella | Q9XDN1 | | pduCDE | Salmonella | P37450 |
| | | enterica | | | | enterica | |
| | AtAdhE | Acetivibrio | A3DCI2 | | pddABC | Klebsiella | Q59470 |
| | | thermocellus | | | | oxytoca | |
| | EcMhpF | Escherichia | P77580 | HACD | BpCaiD_2 | Burkholderia | A0A1X1PK59 |
| | | coli | | | | puraquae | |
| PTA-FOK | CcPta-Ack | Clostridium | A0A0J8D6J2 | | CdHadBC | Clostridioides | Q5U923 |
| | | cylindrosporum | A0A0J8DB00 | | | difficile | |
| | EcPta-Ack | Escherichia | P0A9M8 | ACAD | EcPaaZ | Escherichia | P77455 |
| | | coli | P0A6A3 | | | coli | |
| ACS | EcACS | Escherichia | P27550 | | AtACX4 | Arabidopsis | Q96329 |
| | | coli | | | | thaliana | |
| | StACSstab | Salmonella | Q8ZKF6_PROSS | TER | EgTER | Euglena | Q5EU90 |
| | | typhimurium | | | | gracilis | |
| | MhACS | Marinithermus | F2NQX2 | ECH | CaCRT | Clostridium | P52046 |
| | | hydrothemalis | | | | acetobutylicum | |
| | ArACS | Angustibacter sp. | A0A0Q7JEV7 | | PfECH | Pseudomonas | C3K613 |
| | | Root456 | | | | fluorescens | |
| HACS | RuHACS | Rhodospirillales | A0A1H8YFL8 | ALD | CaHbd | Clostridium | P52041 |
| | | bacterium URHD0017 | | | | acetobutylicum | |
| | BsmHACS | beach sand | A0A3C0TX30 | | EcAldA | Escherichia | P25553 |
| | | metagenome | | | | coli | |
| | DbHACS | Dehalococcoidia | A0A315XEK8 | ACT | CaAbfT | Clostridium | Q9RM86 |
| | | bacterium | | | | aminobutyricum | |
| | AcHACS | Actinomycetospora | P0DUV9 | | OfFrc | Oxalobacter | O06644 |
| | | chiangmaiensis | | | | formigenes | |
| ALDH | EcFrmA | Escherichia | P25437 | FDH | PsFdh | Pseudomonas sp. | P33160 |
| | | coli | | | | (strain 101) | |
| | PpFdhA | Pseudomonas | P46154 | | CbFdh | Candida | O13437 |
| | | putida | | | | boidinii | |
| | | | | TES | EcYciA | Escherichia | P0A8Z0 |
| | | | | | | coli | |

TABLE 13
List of enzymes for carboxylic acid platform using methylgloxyal for the C-1 elongation

| | | | Uniprot | | | | Uniprot |
|---|---|---|---|---|---|---|---|
| Reaction | Gene | Organism | accession | Reaction | Gene | Organism | accession |
| sMMO | mmoXYBZCD | Methylococcus | P18797 | ADH | fucO | Escherichia | P0A9S1 |
| | | capsulatus | | | | coli | |
| pMMO | pmoA1A2B1B2 | Methylococcus | G1UBD1 | | gldA | Escherichia | P0A9S5 |
| | | capsulatus | Q607G3 | | | coli | |
| MDH | BmMDH2 | Bacillus | I3E2P9 | | rhaZ | Salmonella | Q8ZKS2 |
| | | methanolicus MGA3 | | | | typhimurium | |
| | CnMDH2 | Cupriavidus | F8GNE5 | | yahK | Escherichia | P75691 |
| | | necator | | | | coli | |
| | BsMDH | Bacillus | P42327 | | adhA | Lactococcus | Q9CEN0 |
| | | stearothermophilus | | | | lactis | |
| ACR | LmACR | Listeria | Q8Y7U1 | | yjgB | Escherichia | P27250 |
| | | monocytogenes | | | | coli | |
| | StEutE | Salmonella | P41793 | | yqhD | Escherichia | Q46856 |
| | | typhymurium | | | | coli | |
| | CbAld | Clostridium | Q716S8 | | pduQ | Salmonella | Q9XDN0 |
| | | beijerinckii | | | | enterica | |
| | EcMhpF | Escherichia | P77580 | | YLL056C | Saccharomyces | Q12177 |
| | | coli | | | | cerevisiae | |
| | PsDmpF | Pseudomonas sp. | Q52060 | DDR | RiDD | Roseburia | Q1A666 |
| | | strain CF600 | | | | inulinivorans | |
| | pduP | Salmonella | Q9XDN1 | | pduCDE | Salmonella | P37450 |
| | | enterica | | | | enterica | |
| | AtAdhE | Acetivibrio | A3DCI2 | | pddABC | Klebsiella | Q59470 |
| | | thermocellus | | | | oxytoca | |
| | EcMhpF | Escherichia | P77580 | HACD | BpCaiD_2 | Burkholderia | A0A1X1PK59 |
| | | coli | | | | puraquae | |
| PTA-FOK | CcPta-Ack | Clostridium | A0A0J8D6J2 | | CdHadBC | Clostridioides | Q5U923 |
| | | cylindrosporum | A0A0J8DB00 | | | difficile | |
| | EcPta-Ack | Escherichia | P0A9M8 | ACAD | EcPaaZ | Escherichia | P77455 |
| | | coli | P0A6A3 | | | coli | |
| ACS | EcACS | Escherichia | P27550 | | AtACX4 | Arabidopsis | Q96329 |
| | | coli | | | | thaliana | |
| | StACSstab | Salmonella | Q8ZKF6_PROSS | TER | EgTER | Euglena | Q5EU90 |
| | | typhimurium | | | | gracilis | |
| | MhACS | Marinithermus | F2NQX2 | ECH | CaCRT | Clostridium | P52046 |
| | | hydrothemalis | | | | acetobutylicum | |
| | ArACS | Angustibacter sp. | A0A0Q7JEV7 | | PfECH | Pseudomonas | C3K613 |
| | | Root456 | | | | fluorescens | |
| HACS | RuHACS | Rhodospirillales | A0A1H8YFL8 | ALD | CaHbd | Clostridium | P52041 |
| | | bacterium URHD0017 | | | | acetobutylicum | |
| | BsmHACS | beach sand | A0A3C0TX30 | | EcAldA | Escherichia | P25553 |
| | | metagenome | | | | coli | |
| | DbHACS | Dehalococcoidia | A0A315XEK8 | ACT | CaAbfT | Clostridium | Q9RM86 |
| | | bacterium | | | | aminobutyricum | |
| | AcHACS | Actinomycetospora | P0DUV9 | | OfFrc | Oxalobacter | O06644 |
| | | chiangmaiensis | | | | formigenes | |
| ALDH | EcFrmA | Escherichia | P25437 | FDH | PsFdh | Pseudomonas sp. | P33160 |
| | | coli | | | | (strain 101) | |
| | PpFdhA | Pseudomonas | P46154 | | CbFdh | Candida | O13437 |
| | | putida | | | | boidinii | |
| | | | | TES | EcYciA | Escherichia coli | P0A8Z0 |

TABLE 14
List of enzymes for carboxylic acid platform using acetylacetone (pentane-2, 4-dione) for the C-1 elongation

| | | | Uniprot | | | | Uniprot |
|---|---|---|---|---|---|---|---|
| Reaction | Gene | Organism | accession | Reaction | Gene | Organism | accession |
| sMMO | mmoXYBZCD | Methylococcus | P18797 | ADH | fucO | Escherichia | P0A9S1 |
| | | capsulatus | | | | coli | |
| pMMO | pmoA1A2B1B2 | Methylococcus | G1UBD1 | | gldA | Escherichia | P0A9S5 |
| | | capsulatus | Q607G3 | | | coli | |
| MDH | BmMDH2 | Bacillus | I3E2P9 | | rhaZ | Salmonella | Q8ZKS2 |
| | | methanolicus MGA3 | | | | typhimurium | |
| | CnMDH2 | Cupriavidus | F8GNE5 | | yahK | Escherichia | P75691 |
| | | necator | | | | coli | |
| | BsMDH | Bacillus | P42327 | | adhA | Lactococcus | Q9CEN0 |
| | | stearothermophilus | | | | lactis | |
| ACR | LmACR | Listeria | Q8Y7U1 | | yjgB | Escherichia | P27250 |
| | | monocytogenes | | | | coli | |
| | StEutE | Salmonella | P41793 | | yqhD | Escherichia | Q46856 |
| | | typhymurium | | | | coli | |
| | CbAld | Clostridium | Q716S8 | | pduQ | Salmonella | Q9XDN0 |
| | | beijerinckii | | | | enterica | |
| | EcMhpF | Escherichia | P77580 | | YLL056C | Saccharomyces | Q12177 |
| | | coli | | | | cerevisiae | |
| | PsDmpF | Pseudomonas sp. | Q52060 | DDR | RiDD | Roseburia | Q1A666 |
| | | strain CF600 | | | | inulinivorans | |
| | pduP | Salmonella | Q9XDN1 | | pduCDE | Salmonella | P37450 |
| | | enterica | | | | enterica | |
| | AtAdhE | Acetivibrio | A3DCI2 | | pddABC | Klebsiella | Q59470 |
| | | thermocellus | | | | oxytoca | |
| | EcMhpF | Escherichia | P77580 | HACD | BpCaiD_2 | Burkholderia | A0A1X1PK59 |
| | | coli | | | | puraquae | |
| PTA-FOK | CcPta-Ack | Clostridium | A0A0J8D6J2 | | CdHadBC | Clostridioides | Q5U923 |
| | | cylindrosporum | A0A0J8DB00 | | | difficile | |
| | EcPta-Ack | Escherichia | P0A9M8 | ACAD | EcPaaZ | Escherichia | P77455 |
| | | coli | P0A6A3 | | | coli | |
| ACS | EcACS | Escherichia coli | P27550 | | AtACX4 | Arabidopsis thaliana | Q96329 |
| | StACSstab | Salmonella | Q8ZKF6_PROSS | TER | EgTER | Euglena | Q5EU90 |
| | | typhimurium | | | | gracilis | |
| | MhACS | Marinithermus | F2NQX2 | ECH | CaCRT | Clostridium | P52046 |
| | | hydrothemalis | | | | acetobutylicum | |
| | ArACS | Angustibacter sp. | A0A0Q7JEV7 | | PfECH | Pseudomonas | C3K613 |
| | | Root456 | | | | fluorescens | |
| HACS | RuHACS | Rhodospirillales | A0A1H8YFL8 | ALD | CaHbd | Clostridium | P52041 |
| | | bacterium URHD0017 | | | | acetobutylicum | |
| | BsmHACS | beach sand | A0A3C0TX30 | | EcAldA | Escherichia | P25553 |
| | | metagenome | | | | coli | |
| | DbHACS | Dehalococcoidia | A0A315XEK8 | ACT | CaAbfT | Clostridium | Q9RM86 |
| | | bacterium | | | | aminobutyricum | |
| | AcHACS | Actinomycetospora | P0DUV9 | | OfFrc | Oxalobacter | O06644 |
| | | chiangmaiensis | | | | formigenes | |
| ALDH | EcFrmA | Escherichia | P25437 | FDH | PsFdh | Pseudomonas sp. | P33160 |
| | | coli | | | | (strain 101) | |
| | PpFdhA | Pseudomonas | P46154 | | CbFdh | Candida | O13437 |
| | | putida | | | | boidinii | |
| | | | | TES | EcYciA | Escherichia coli | P0A8Z0 |

TABLE 15
Description of enzyme abbreviation for each reaction step

| Reaction | Enzyme |
|---|---|
| MMO | Methane monooxygenase |
| MDH | Methanol dehydrogenase |
| FLDH | Formaldehyde dehydrogenase |
| FDH | Formate dehydrogenase |
| FOK | Formate kinase |
| PTA | Phosphotransacylase |
| ACR | Acyl-CoA reductase |
| ACS | Acyl-CoA synthase |
| ACT | Acyl-CoA transferase |
| CAK | Carboxylic acid kinase |
| HACS | 2-Hydroxyacyl-CoA synthase |
| HACD | 2-Hydroxyacyl-CoA dehydratase |
| TES | Thioesterase |
| ADH | Alcohol dehydrogenase |
| ECH | Enoyl-CoA hydratase |
| TER | Trans-2-enoyl-CoA reductase |
| DDR | Diol dehydratase |
| HADH | 2-Hydroxyacyl-CoA dehydrogenase |
| AADH | Amino acid dehydrogenase |
| TA | Transaminase |
| ALDH | Aldehyde dehydrogenase |

TABLE 16
R group for C₁ carboxylic acids and corresponding
aldehydes for CA platform

| | Carboxylic acid | Aldehyde |
|---|---|---|
| R group | Compound and Structure | Compound and Structure |
| H | 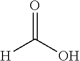 | 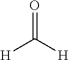 |
| | Formic acid | Formaldehyde |
| NH₂ | 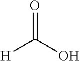 | 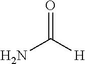 |
| | Formic acid | Formamide |
| Phenyl | 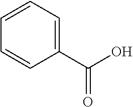 | 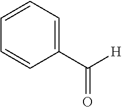 |
| | Benzoic acid | Benzaldehyde |

TABLE 17
R group for C₂₊ carboxylic acids and corresponding aldehydes for CA platform

| | Carboxylic acid | Aldehyde |
|---|---|---|
| R group | Compound and Structure | Compound and Structure |
| H | 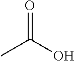 | 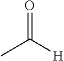 |
| | Acetic acid | Acetaldehyde |
| CH₃ | 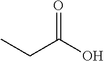 | 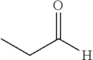 |
| | Propionic acid | Propionaldehyde |
| CH₂CH₃ | 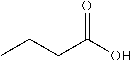 | 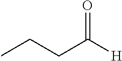 |
| | Butyric acid | Butyraldehyde |
| (CH₂)₂CH₃ | 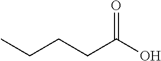 | 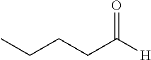 |
| | Pentanoic acid | Valeraldehyde |
| (CH₂)₃CH₃ | 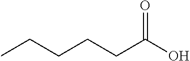 | 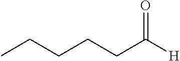 |
| | Hexanoic acid | Hexanal |
| (CH₂)₄CH₃ | 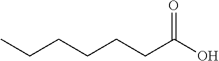 | 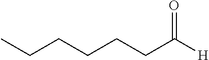 |
| | Heptanoic acid | Heptanal |
| (CH₂)₅CH₃ | 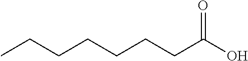 | 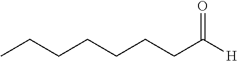 |
| | Octanoic acid | Octanal |
| (CH₂)₆CH₃ | 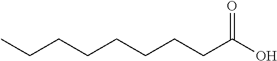 | 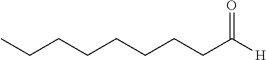 |
| | Nonanoic acid | Nonanal |
| OH | 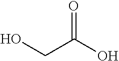 | 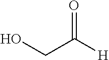 |
| | Glycolic acid | Glycolaldehyde |
| CH₃OH | 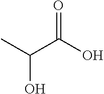 | 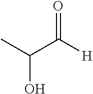 |
| | Lactic acid | Lactaldehyde |
| OHCH₂OH | 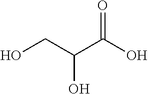 | 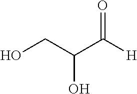 |
| | Glyceric acid | Glyceraldehyde |
| OOH | 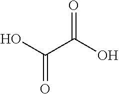 | 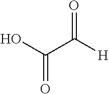 |
| | Oxalic acid | Glyoxylic acid |
| CH₂OH | 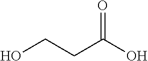 | 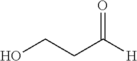 |
| | 3-Hydroxypropionic acid | 3-Hydroxypropionaldehyde |
| COOH | 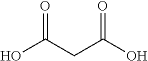 | 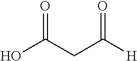 |
| | Malonic acid | Malonic semialdehyde |
| CH₂COOH | 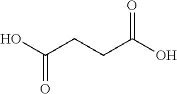 | 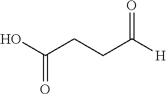 |
| | Succinic acid | Succinic semialdehyde |
| (CH₃)₂ | 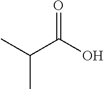 | 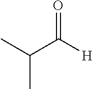 |
| | Isobutyric acid | Isobutyraldehyde |
| CH(CH₃)₂ | 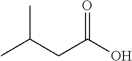 | 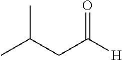 |
| | Isovaleric acid | Isovaleraldehyde |
| Phenyl | 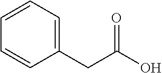 | 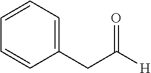 |
| | Phenylacetic acid | Phenylacetaldehyde |

TABLE 18
R₁ and R₂ groups for carboxylic acids and corresponding ketones for CA platform

| | R₂ | Carboxylic acid | Ketone |
|---|---|---|---|
| R₁ group | group | Compound and Structure | Compound and Structure |
| H | CH₃ | 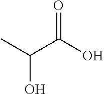 | 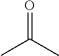 |
| | | Lactic acid | Acetone |
| CH₃ | CH₃ | 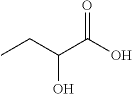 | 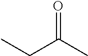 |
| | | 2-Hydroxybutyric acid | 2-Butanone |
| CH₂CH₃ | CH₃ | 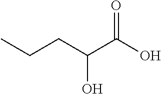 | 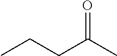 |
| | | 2-Hydroxypentanoic acid | 2-Pentanone |
| (CH₂)₃CH₃ | CH₃ | 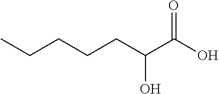 | 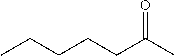 |
| | | 2-Hydroxyheptanoic acid | 2-Heptanone |
| OH | CH₃ | 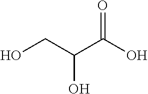 | 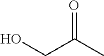 |
| | | 2,3-Dihydroxypropionic acid | Hydroxyacetone |
| (CH₃)₂ | CH₃ | 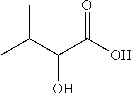 | 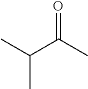 |
| | | 2-Hydroxyisovaleric acid | 3-Methyl-2-butanone |
| O | CH₃ | 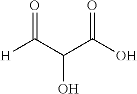 | 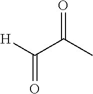 |
| | | Tartronic semialdehyde | Methylglyoxal |
| COCH₃ | CH₃ | 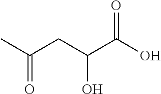 | 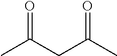 |
| | | 2-Hydroxy-4-oxopentanoic acid | Acetylacetone |

Experimental methods are similar to those described in the above examples.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A genetically modified microorganism converting a carboxylic acid to a product, comprising:
a. a first set of nucleic acids encoding enzymes to activate said carboxylic acid to the corresponding acyl-CoA intermediate;
b. a second set of nucleic acids encoding enzymes to convert said acyl-CoA intermediate to a product; and
c. a third set of nucleic acids encoding enzymes to generate reducing equivalents and ATP from an externally supplied energy source.

2. The microorganism of claim 1, wherein said first set of metabolic enzymes comprises an acyl-CoA synthetase, or an acyl-CoA transferase, or a carboxylate kinase and a phosphotransacylase, or a carboxylic acid reductase and an acyl-CoA reductase, or an aldehyde dehydrogenase and an acyl-CoA reductase converting said carboxylic acid to the corresponding acyl-CoA intermediate.

3. The microorganism of claim 1, wherein said second set of metabolic enzymes comprises an aldehyde forming acyl-CoA reductase converting said acyl-CoA to an aldehyde; or wherein said second set of metabolic enzymes comprises an aldehyde forming acyl-CoA reductase and alcohol dehydrogenase, or an alcohol forming acyl-CoA reductase converting said acyl-CoA to an alcohol.

4. The microorganism of claim 3, wherein said carboxylic acid is a 2-hydroxyacid and said aldehyde is a 2-hydroxyaldehyde.

5. (canceled)

6. The microorganism of claim 3, wherein said carboxylic acid is a 2-hydroxyacid and said alcohol is a 1,2-diol.

7. The microorganism of claim 6, wherein said second set of metabolic enzymes further comprises a diol dehydratase converting said 1,2-diol to a ketone; or wherein said second set of metabolic enzymes further comprises a diol dehydratase converting said 1,2-diol to an aldehyde.

8. (canceled)

9. The microorganism of claim 7, wherein said second set of metabolic enzymes further comprises an alcohol dehydrogenase converting said aldehyde to a primary alcohol; or wherein said second set of metabolic enzymes further comprises an acylating aldehyde dehydrogenase converting said aldehyde to an acyl-CoA and a thioesterase, or an acyl-CoA transferase, or a phosphotransacylase and a carboxylate kinase converting said acyl-CoA to a carboxylic acid.

10. (canceled)

11. The microorganism of claim 3, wherein said second set of metabolic enzymes further comprises enzymes to convert a 1-carbon substrate to formyl-CoA and a 2-hydroxyacyl-CoA lyase or oxalyl-CoA decarboxylase condensing said aldehyde with said formyl-CoA to form a 2-hydroxyacyl-CoA 1 carbon longer than said aldehyde.

12. The microorganism of claim 11, wherein said second set of metabolic enzymes further comprises a thioesterase, or an acyl-CoA transferase, or a phosphotransacylase and a carboxylate kinase converting said 2-hydroxyacyl-CoA to a 2-hydroxyacid; or wherein said second set of metabolic enzymes further comprises an acyl-CoA reductase converting said 2-hydroxyacyl-CoA to a 2-hydroxyaldehyde; or wherein said second set of metabolic enzymes further comprises an alcohol-forming acyl-CoA reductase converting said 2-hydroxyacyl-CoA to a 1,2-diol.

13. (canceled)

14. The microorganism of claim 12, wherein said second set of metabolic enzymes further comprises an alcohol dehydrogenase converting said 2-hydroxyaldehyde to a 1,2-diol.

15. (canceled)

16. The microorganism of claim 12, wherein said second set of metabolic enzymes further comprises a diol dehydratase converting said 1,2-diol to an aldehyde.

17. The microorganism of claim 16, wherein said second set of metabolic enzymes further comprises an alcohol dehydrogenase converting said aldehyde to a primary alcohol.

18. The microorganism of claim 4, wherein said second set of metabolic enzymes further comprises enzymes to convert a 1-carbon substrate to formyl-CoA and a 2-hydroxyacyl-CoA lyase or oxalyl-CoA decarboxylase condensing said 2-hydroxyaldehyde with said formyl-CoA to form a 2,3-dihydroxyacyl-CoA 1 carbon longer than said 2-hydroxyaldehyde.

19. The microorganism of claim 18, wherein said second set of metabolic enzymes further comprises a thioesterase, or an acyl-CoA transferase, or a phosphotransacylase and a carboxylate kinase converting said 2,3-dihydroxyacyl-CoA to a 2,3-dihydroxyacid; or wherein said second set of metabolic enzymes further comprises an acyl-CoA reductase converting said 2,3-dihydroxyacyl-CoA to a 2,3-dihydroxyaldehyde; or wherein said second set of metabolic enzymes further comprises an alcohol-forming acyl-CoA reductase converting said 2-hydroxyacyl-CoA to a 1,2-diol.

20. (canceled)

21. The microorganism of claim 19, wherein said second set of metabolic enzymes further comprises an alcohol dehydrogenase converting said 2,3-dihydroxyacyl-CoA to a 1,2,3-triol.

22. (canceled)

23. The microorganism of claim 21, wherein said second set of metabolic enzymes further comprises a diol dehydratase converting said 1,2,3-triol to a 3-hydroxyaldehyde.

24. The microorganism of claim 23, wherein said second set of metabolic enzymes further comprises an alcohol dehydrogenase converting said 3-hydroxyaldehyde to a 1,3-diol.

25. The microorganism of claim 7, wherein said second set of metabolic enzymes further comprises enzymes to convert a 1-carbon substrate to formyl-CoA and a 2-hydroxyacyl-CoA lyase or oxalyl-CoA decarboxylase condensing said ketone with said formyl-CoA to form a 2-methyl-2-hydroxyacyl-CoA 1 carbon longer than said ketone.

26. The microorganism of claim 25, wherein said second set of metabolic enzymes further comprises a thioesterase, or an acyl-CoA transferase, or a phosphotransacylase and a carboxylate kinase converting said 2-methyl-2-hydroxyacyl-CoA to a 2-methyl-2-hydroxyacid; or wherein said second set of metabolic enzymes further comprises an acyl-CoA reductase converting said 2-methyl-2-hydroxyacyl-CoA to a 2-methyl-2-hydroxyaldehyde.

27. (canceled)

28. The microorganism of claim 26, wherein said second set of metabolic enzymes further comprises an alcohol dehydrogenase converting said 2-methyl-2-hydroxyaldehyde to a 2-methyl-1,2-diol.

29. The microorganism of claim 25, wherein said second set of metabolic enzymes further comprises an alcohol-forming acyl-CoA reductase converting said 2-methyl-2-hydroxyacyl-CoA to a 2-methyl-1,2-diol.

30. The microorganism of claim 28, wherein said second set of metabolic enzymes further comprises a diol dehydratase converting said 2-methyl-1,2-diol to a 2-methyl-aldehyde.

31. The microorganism of claim 30, wherein said second set of metabolic enzymes further comprises an alcohol dehydrogenase converting said 2-methyl-aldehyde to a 2-methyl primary alcohol.

32. The microorganism of claim 8, wherein said second set of metabolic enzymes further comprises enzymes to convert a 1-carbon substrate to formyl-CoA and a 2-hydroxyacyl-CoA lyase or oxalyl-CoA decarboxylase condensing said aldehyde with said formyl-CoA to form a 2-hydroxyacyl-CoA 1 carbon longer than said aldehyde.

33. The microorganism of claim 32, wherein said second set of metabolic enzymes further comprises a thioesterase, or an acyl-CoA transferase, or a phosphotransacylase and a carboxylate kinase converting said 2-hydroxyacyl-CoA to a 2-hydroxyacid; or wherein said second set of metabolic enzymes further comprises an acyl-CoA reductase converting said 2-hydroxyacyl-CoA to a 2-hydroxyaldehyde; or wherein said second set of metabolic enzymes further comprises an alcohol-forming acyl-CoA reductase converting said 2-hydroxyacyl-CoA to a 1,2-diol.

34. (canceled)

35. The microorganism of claim 33, wherein said second set of metabolic enzymes further comprises an alcohol dehydrogenase converting said 2-hydroxyaldehyde to a 1,2-diol.

36. (canceled)

37. The microorganism of claim 35, wherein said second set of metabolic enzymes further comprises a diol dehydratase converting said 1,2-diol to an aldehyde.

38. The microorganism of claim 37, wherein said second set of metabolic enzymes further comprises an alcohol dehydrogenase converting said aldehyde to a primary alcohol.

39. The microorganism of claim 11, wherein said enzymes to convert a 1-carbon substrate to formyl-CoA comprise a methanol dehydrogenase converting methanol to formaldehyde and an acyl-CoA reductase converting formaldehyde to formyl-CoA and said 1-carbon substrate is methanol;
wherein said enzymes to convert a 1-carbon substrate to formyl-CoA comprise an acyl-CoA reductase converting formaldehyde to formyl-CoA and said 1-carbon substrate is formaldehyde; or
wherein said enzymes to convert a 1-carbon substrate to formyl-CoA comprise an acyl-CoA synthetase, or an acyl-CoA transferase, or a carboxylate kinase and a phosphotransacylase, or a carboxylic acid reductase and an acyl-CoA reductase, or an aldehyde dehydrogenase and an acyl-CoA reductase converting formate to formyl-CoA and said 1-carbon substrate is formate.

40. (canceled)

41. (canceled)

42. The microorganism of claim 1, wherein said externally supplied energy source is a reduced 1-carbon substrate.

43. The microorganism of claim 42, wherein said reduced 1-carbon substrate is selected from the group consisting of methanol and formaldehyde.

44. The microorganism of claim 43, wherein said third set of metabolic enzymes comprises methanol dehydrogenase converting methanol to formaldehyde, a formaldehyde dehydrogenase converting formaldehyde to formate, and a formate dehydrogenase converting formate to CO2; or
wherein said third set of metabolic enzymes comprises a formaldehyde dehydrogenase converting formaldehyde to formate and a formate dehydrogenase converting formate to CO2.

45. (canceled)

46. (canceled)

47. The microorganism of claim 42, wherein said third set of metabolic enzymes further comprises an acylating formaldehyde dehydrogenase converting formaldehyde to formyl-CoA, a phosphate formyltransferase converting formyl-CoA to formyl-phosphate, and a formate kinase converting formyl-phosphate to formate.

48. The microorganism of claim 42, wherein said third set of metabolic enzymes further comprises enzymes generating ATP from NADH.

49. The microorganism of claim 1, the microorganism further comprising metabolic enzymes allowing the generation of a carboxylic acid from a carbon feedstock.

50. The microorganism of claim 49, wherein said carbon feedstock is selected from the group consisting of glucose, xylose, arabinose, glycerol, methane, CO2, methanol, formate, formaldehyde, and similar substances.

51. The microorganism of claim 1, the microorganism being bacteria or yeast.

52. A method of culturing the microorganism of claim 1, comprising incubating the microorganism with a carboxylic acid and external energy source under suitable conditions such that the carboxylic acid is converted to a reduced product of interest, and optionally further comprising isolating a product of interest from the microbial culture.

53. A method of culturing the microorganism of claim 1, comprising incubating the microorganism with a carbon feedstock and external energy source under suitable conditions such that the carbon feedstock is converted to a reduced product of interest, and optionally further comprising isolating a product of interest from the microbial culture.

54. (canceled)
